SEQUENCE LISTING (180 sequences)

SEQ ID NO: 1 - protein, 37 aa, unidentified
  REGION 1..37: note = Parabuthus transvaalicus toxin (PaT1)
QMDMRCSASVECKQKCLKAIGSIFGKCMNKKCKCYPR

SEQ ID NO: 2 - protein, 37 aa, unidentified
  REGION 1..37: note = Parabuthus transvaalicus toxin (PaT2)
QMDMRCSASVECKQKCLKAIGRGFGKCMNKKCKCYPR

SEQ ID NO: 3 - protein, 37 aa, synthetic construct
  REGION 1..37: note = Modified SEQ ID NO 1
NMDMRCSASVECKQKCLKAIGSIFGKCMNKKCKCYPR

SEQ ID NO: 4 - protein, 37 aa, synthetic construct
  REGION 1..37: note = Modified SEQ ID NO 1
NMDMRCSASVECKQKCLKAIGRGFGKCMNKKCKCYPR

SEQ ID NO: 5 - protein, 36 aa, synthetic construct
  REGION 1..36: note = Modified SEQ ID NO 1
MDMRCSASVECKQKCLKAIGSIFGKCMNKKCKCYPR

SEQ ID NO: 6 - protein, 35 aa, synthetic construct
  REGION 1..35: note = Modified SEQ ID NO 1
DMRCSASVECKQKCLKAIGSIFGKCMNKKCKCYPR

SEQ ID NO: 7 - protein, 37 aa, synthetic construct; identical to SEQ ID NO: 3
  REGION 1..37: note = Modified SEQ ID NO 1

SEQ ID NO: 8 - protein, 37 aa, synthetic construct
  REGION 1..37: note = Modified SEQ ID NO 1
NIDMRCSASVECKQKCLKAIGSIFGKCMNKKCKCYPR

SEQ ID NO: 9 - protein, 37 aa, synthetic construct
  REGION 1..37: note = Modified SEQ ID NO 1
NMDVRCSASVECKQKCLKAIGSIFGKCMNKKCKCYPR

SEQ ID NO: 10 - protein, 37 aa, synthetic construct
  REGION 1..37: note = Modified SEQ ID NO 1
NMDMRCSASVECKQKCKDAIGSIFGKCMNKKCKCYPR

SEQ ID NO: 11 - protein, 39 aa, synthetic construct
  REGION 1..39: note = Modified SEQ ID NO 1
GGNMDMRCSASVECKQKCLKAIGSIFGKCMNKKCKCYPR

SEQ ID NO: 12 - protein, 37 aa, synthetic construct
  REGION 1..37: note = Modified SEQ ID NO 1
NMEMRCSASVECKQKCLKAIGSIFGKCMNKKCKCYPR

SEQ ID NO: 13 - protein, 39 aa, synthetic construct
  REGION 1..39: note = Modified SEQ ID NO 1
SGNMDMRCSASVECKQKCLKAIGSIFGKCMNKKCKCYPR

SEQ ID NO: 14 - protein, 37 aa, synthetic construct
  REGION 1..37: note = Modified SEQ ID NO 1
  SITE 2: note = MISC_FEATURE - Xaa is norleucine
NXDMRCSASVECKQKCLKAIGSIFGKCMNKKCKCYPR

SEQ ID NO: 15 - protein, 37 aa, synthetic construct
  REGION 1..37: note = Modified SEQ ID NO 1
  SITE 4: note = MISC_FEATURE - Xaa is norleucine
NMDXRCSASVECKQKCLKAIGSIFGKCMNKKCKCYPR

SEQ ID NO: 16 - protein, 37 aa, synthetic construct
  REGION 1..37: note = Modified SEQ ID NO 1
  SITE 28: note = MISC_FEATURE - Xaa is norleucine
NMDMRCSASVECKQKCLKAIGSIFGKCXNKKCKCYPR

SEQ ID NO: 17 - protein, 37 aa, synthetic construct
  REGION 1..37: note = Modified SEQ ID NO 1
NMDMRCSASVECKVKCLKAIGSIFGKCMNKKCKCYPR

SEQ ID NO: 18 - protein, 37 aa, synthetic construct
  REGION 1..37: note = Modified SEQ ID NO 1
NMDMRCSASVECKQLCLKAIGSIFGKCMNKKCKCYPR

SEQ ID NO: 19 - protein, 37 aa, synthetic construct
  REGION 1..37: note = Modified SEQ ID NO 1
NMDMRCSASVECKQKCKKAIGSIFGKCMNKKCKCYPR

SEQ ID NO: 20 - protein, 37 aa, synthetic construct
  REGION 1..37: note = Modified SEQ ID NO 1
NMDMRCSASVECKQKCLDAIGSIFGKCMNKKCKCYPR

SEQ ID NO: 21 - protein, 37 aa, synthetic construct
  REGION 1..37: note = Modified SEQ ID NO 1
NMDMRCSASVECKQKCLKAIRSIFGKCMNKKCKCYPR

SEQ ID NO: 22 - protein, 37 aa, synthetic construct
  REGION 1..37: note = Modified SEQ ID NO 1
NMDMRCSASVECKQKCLKAIESIFGKCMNKKCKCYPR

SEQ ID NO: 23 - protein, 41 aa, synthetic construct
  REGION 1..41: note = Modified SEQ ID NO 1
NMDMRCSASVECKQKCLKAIGSIFGKCMNKKCKCYPRRRTA

SEQ ID NO: 24 - protein, 41 aa, synthetic construct
  REGION 1..41: note = Modified SEQ ID NO 1
NMDMRCSASVECKQKCLKAIGSIFGKCMNKKCKCYPRHRRK

SEQ ID NO: 25 - protein, 37 aa, synthetic construct
  REGION 1..37: note = Modified SEQ ID NO 1
NMDMRCKASVECKQKCLKAIGSIFGKCMNKKCKCYPR

SEQ ID NO: 26 - protein, 37 aa, synthetic construct
  REGION 1..37: note = Modified SEQ ID NO 1
NMDMRCSISVECKQKCLKAIGSIFGKCMNKKCKCYPR

SEQ ID NO: 27 - protein, 37 aa, synthetic construct
  REGION 1..37: note = Modified SEQ ID NO 1
NMDMRCSASRECKQKCLKAIGSIFGKCMNKKCKCYPR

SEQ ID NO: 28 - protein, 37 aa, synthetic construct
  REGION 1..37: note = Modified SEQ ID NO 1
NMDMRCSASVQCKQKCLKAIGSIFGKCMNKKCKCYPR

SEQ ID NO: 29 - protein, 37 aa, synthetic construct
  REGION 1..37: note = Modified SEQ ID NO 1
NMDMRCSASVECLQKCLKAIGSIFGKCMNKKCKCYPR

SEQ ID NO: 30 - protein, 37 aa, synthetic construct
  REGION 1..37: note = Modified SEQ ID NO 1
NMDMRCSASVECAQKCLKAIGSIFGKCMNKKCKCYPR

SEQ ID NO: 31 - protein, 37 aa, synthetic construct
  REGION 1..37: note = Modified SEQ ID NO 1
NMDMRCSASVECKEKCLKAIGSIFGKCMNKKCKCYPR

SEQ ID NO: 32 - protein, 37 aa, synthetic construct
  REGION 1..37: note = Modified SEQ ID NO 1
NMDMRCSASVECKLKCLKAIGSIFGKCMNKKCKCYPR

SEQ ID NO: 33 - protein, 37 aa, synthetic construct
  REGION 1..37: note = Modified SEQ ID NO 1
NMDMRCSASVECKQKCLKAIHSIFGKCMNKKCKCYPR

SEQ ID NO: 34 - protein, 37 aa, synthetic construct
  REGION 1..37: note = Modified SEQ ID NO 1
NMDMRCSASVECKQKCLKAIGSKFGKCMNKKCKCYPR

SEQ ID NO: 35 - protein, 37 aa, synthetic construct
  REGION 1..37: note = Modified SEQ ID NO 1
NMDMRCSASVECKQKCLKAIGSRFGKCMNKKCKCYPR

SEQ ID NO: 36 - protein, 37 aa, synthetic construct
  REGION 1..37: note = Modified SEQ ID NO 1
NMDMRCSASVECKQKCLKAIGSIFGKCMNGKCKCYPR

SEQ ID NO: 37 - protein, 37 aa, synthetic construct
  REGION 1..37: note = Modified SEQ ID NO 1
NMDMRCSASVECKQKCLKAIGSIFGKCMNKKCHCYPR

SEQ ID NO: 38 - protein, 37 aa, synthetic construct
  REGION 1..37: note = Modified SEQ ID NO 1
NMDMRCSASVECKQKCLKAIGSIFGKCMNKKCVCYPR

SEQ ID NO: 39 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
NXDXRCSASVECKQKCLKAIGSIFGKCMNKKCKCYPR

SEQ ID NO: 40 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
NXDXRCSASVECKQKCLKAIGSIFGKCXNKKCKCYPR

SEQ ID NO: 41 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
PXDXRCSASVECKQKCLKAIGSIFGKCXNKKCKCYPR

SEQ ID NO: 42 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
NXDXRCRASVECKQKCLKAIGSIFGKCXNKKCKCYPR

SEQ ID NO: 43 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
NXDXRCSASVECEQKCLKAIGSIFGKCXNKKCKCYPR

SEQ ID NO: 44 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
NXDXRCSASVECQQKCLKAIGSIFGKCXNKKCKCYPR

SEQ ID NO: 45 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
NXDXRCSASVECKKKCLKAIGSIFGKCXNKKCKCYPR

SEQ ID NO: 46 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
NXSXRCSASVECKQKCLKAIGSIFGKCXNKKCKCYPR

SEQ ID NO: 47 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
NXDXRCSHSVECKQKCLKAIGSIFGKCXNKKCKCYPR

SEQ ID NO: 48 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
NXDXRCSASVECKQSCLKAIGSIFGKCXNKKCKCYPR

SEQ ID NO: 49 - protein, 36 aa, synthetic construct
  REGION 1..36: note = Modified SEQ ID NO 1
  SITE 2: note = MISC_FEATURE - Xaa is norleucine
  SITE 4: note = MISC_FEATURE - Xaa is norleucine
  SITE 27: note = MISC_FEATURE - Xaa is norleucine
NXDXRCSASVECKQKCKAIGSIFGKCXNKKCKCYPR

SEQ ID NO: 50 - protein, 37 aa, synthetic construct
  REGION 1..37: note = Modified EQ ID NO 1
NMDMRCSASVECKQKCYKAIGSIFGKCMNKKCKCYPR

SEQ ID NO: 51 - protein, 37 aa, synthetic construct
  REGION 1..37: note = Modified SEQ ID NO 1
NMDMRCSASVECKQKCRKAIGSIFGKCMNKKCKCYPR

SEQ ID NO: 52 - protein, 37 aa, synthetic construct
  REGION 1..37: note = Modified SEQ ID NO 1
NMDMRCSASVECKQKCLAAIGSIFGKCMNKKCKCYPR

SEQ ID NO: 53 - protein, 37 aa, synthetic construct
  REGION 1..37: note = Modified SEQ ID NO 1
NMDMRCSASVECKQKCLYAIGSIFGKCMNKKCKCYPR

SEQ ID NO: 54 - protein, 36 aa, synthetic construct
  REGION 1..36: note = Modified SEQ ID NO 1
NMDMRCSASVECKQKCLAIGSIFGKCMNKKCKCYPR

SEQ ID NO: 55 - protein, 37 aa, synthetic construct
  REGION 1..37: note = Modified SEQ ID NO 1
NMDMRCSASVECKQKCLKYIGSIFGKCMNKKCKCYPR

SEQ ID NO: 56 - protein, 37 aa, synthetic construct
  REGION 1..37: note = Modified SEQ ID NO 1
NMDMRCSASVECKQKCLKRIGSIFGKCMNKKCKCYPR

SEQ ID NO: 57 - protein, 36 aa, synthetic construct
  REGION 1..36: note = Modified SEQ ID NO 1
NMDMRCSASVECKQKCLKIGSIFGKCMNKKCKCYPR

SEQ ID NO: 58 - protein, 37 aa, synthetic construct
  REGION 1..37: note = Modified SEQ ID NO 1
NMDMRCSASVECKQKCLKAYGSIFGKCMNKKCKCYPR

SEQ ID NO: 59 - protein, 37 aa, synthetic construct
  REGION 1..37: note = Modified SEQ ID NO 1
NMDMRCSASVECKQKCLKAEGSIFGKCMNKKCKCYPR

SEQ ID NO: 60 - protein, 37 aa, synthetic construct
  REGION 1..37: note = Modified SEQ ID NO 1
NMDMRCSASVECKQKCLKARGSIFGKCMNKKCKCYPR

SEQ ID NO: 61 - protein, 36 aa, synthetic construct
  REGION 1..36: note = Modified SEQ ID NO 1
NMDMRCSASVECKQKCLKAGSIFGKCMNKKCKCYPR

SEQ ID NO: 62 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
NXDXRCSASVECKQKCLKAIGSPFGKCXNKKCKCYPR

SEQ ID NO: 63 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
NXDXRCSASKECKQKCLKAIGSIFGKCXNKKCKCYPR

SEQ ID NO: 64 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
HXDXRCSASVECKQKCLKAIGSIFGKCXNKKCKCYPR

SEQ ID NO: 65 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
YXDXRCSASVECKQKCLKAIGSIFGKCXNKKCKCYPR

SEQ ID NO: 66 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
SXDXRCSASVECKQKCLKAIGSIFGKCXNKKCKCYPR

SEQ ID NO: 67 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
VXDXRCSASVECKQKCLKAIGSIFGKCXNKKCKCYPR

SEQ ID NO: 68 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 9: note = MISC_FEATURE - Xaa is alpha-aminobutyric acid (homo-alanine)
SXDXRCSAXVECKQKCLKAIGSIFGKCMNKKCKCYPR

SEQ ID NO: 69 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 26: note = MISC_FEATURE - Xaa is homo-lysine
SXDXRCSASVECKQKCLKAIGSIFGXCMNKKCKCYPR

SEQ ID NO: 70 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 35: note = MISC_FEATURE - Xaa is 4-amino-phenylalanine
SXDXRCSASVECKQKCLKAIGSIFGKCMNKKCKCXPR

SEQ ID NO: 71 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
SXDXRCSASVECGQKCLKAIGSIFGKCMNKKCKCYPR

SEQ ID NO: 72 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
SXDXRCSASVECVQKCLKAIGSIFGKCMNKKCKCYPR

SEQ ID NO: 73 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 14: note = MISC_FEATURE - Xaa is 2-amino-5-carboxypentanoyl
SXDXRCSASVECKXKCLKAIGSIFGKCMNKKCKCYPR

SEQ ID NO: 74 - protein, 36 aa, synthetic construct
  REGION 1..36: note = Modified SEQ ID NO 1
  SITE 2: note = MISC_FEATURE - Xaa is norleucine
  SITE 4: note = MISC_FEATURE - Xaa is norleucine
SXDXRCSASVECKQKCLKAIGSIFGKCMNKKCKCYQ

SEQ ID NO: 75 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
SXDXRCSASVECKQKCLKAIGSIFGKCMNKKCRCYPR

SEQ ID NO: 76 - protein, 37 aa, synthetic construct
  REGION 1..37: note = Modified SEQ ID NO 1
  SITE 2: note = MISC_FEATURE - Xaa is norleucine
SXDERCSASVECKQKCLKAIGSIFGKCMNKKCKCYPR

SEQ ID NO: 77 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
SXDXRCSASVECKQKCLGAIGSIFGKCMNKKCKCYPR

SEQ ID NO: 78 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
SXDXRCSASVECKQKCLVAIGSIFGKCMNKKCKCYPR

SEQ ID NO: 79 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
SXDXRCSASVECAQSCLKAIGSIFGKCMNKKCKCYPR

SEQ ID NO: 80 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
SXDXRCSASVECAQKCLAAIGSIFGKCMNKKCKCYPR

SEQ ID NO: 81 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
SXDXRCSASVECAQLCLAAIGSIFGKCMNKKCKCYPR

SEQ ID NO: 82 - protein, 37 aa, synthetic construct; identical to SEQ ID NO: 66
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine

SEQ ID NO: 83 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
PXDXRCSASVECKQKCLKAIGCIFGKCXNKKCKCYPC

SEQ ID NO: 84 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
SXDXRCSASVECKEKCLQAIGSIFGKCXNKKCKCYPR

SEQ ID NO: 85 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 18: note = MISC_FEATURE - Xaa is homo-glutamine
  SITE 28: note = MISC_FEATURE - Xaa is norleucine
PXDXRCSASVECKEKCLXAIGSIFGKCXNKKCKCYPR

SEQ ID NO: 86 - protein, 32 aa, synthetic construct
  REGION 1..32: note = Modified SEQ ID NO 1
  SITE 23: note = MISC_FEATURE - Xaa is norleucine
CSASVECKQKCLKAIGCIFGKCXNKKCKCYPC

SEQ ID NO: 87 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
SXDXRCSASVECKQKCLAAIGCIFGKCXNKKCKCYPC

SEQ ID NO: 88 - protein, 37 aa, synthetic construct; identical to SEQ ID NO: 83
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine

SEQ ID NO: 89 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
SXDXRCSALVECKQKCLKAIGSIFGKCMNKKCKCYPR

SEQ ID NO: 90 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
SXDXRCSAVVECKQKCLKAIGSIFGKCMNKKCKCYPR

SEQ ID NO: 91 - protein, 37 aa, synthetic construct; identical to SEQ ID NO: 70
  REGION 1..37: note = Modified SEQ ID NO 1
  SITE 2: note = MISC_FEATURE - Xaa is norleucine
  SITE 4: note = MISC_FEATURE - Xaa is norleucine
  SITE 35: note = MISC_FEATURE - Xaa is 4-fluoro-phenylalanine

SEQ ID NO: 92 - protein, 37 aa, synthetic construct; identical to SEQ ID NO: 70
  REGION 1..37: note = Modified SEQ ID NO 1
  SITE 2: note = MISC_FEATURE - Xaa is norleucine
  SITE 4: note = MISC_FEATURE - Xaa is norleucine
  SITE 35: note = MISC_FEATURE - Xaa is 4-nitro-phenylalanine

SEQ ID NO: 93 - protein, 37 aa, synthetic construct; identical to SEQ ID NO: 70
  REGION 1..37: note = Modified SEQ ID NO 1
  SITE 2: note = MISC_FEATURE - Xaa is norleucine
  SITE 4: note = MISC_FEATURE - Xaa is norleucine
  SITE 35: note = MISC_FEATURE - Xaa is 4-methyl-phenylalanine

SEQ ID NO: 94 - protein, 36 aa, synthetic construct
  REGION 1..36: note = Modified SEQ ID NO 1
  SITE 1: note = MISC_FEATURE - Xaa is norleucine
  SITE 3: note = MISC_FEATURE - Xaa is norleucine
  SITE 27: note = MISC_FEATURE - Xaa is norleucine
XDXRCSASVECKQKCLKAIGSIFGKCXNKKCKCYPR

SEQ ID NO: 95 - protein, 37 aa, synthetic construct
  REGION 1..37: note = Modified SEQ ID NO 1
  SITE 4: note = MISC_FEATURE - Xaa is norleucine
  SITE 28: note = MISC_FEATURE - Xaa is norleucine
NIDXRCSASVECKQKCLKAIGSIFGKCXNKKCKCYPR

SEQ ID NO: 96 - protein, 37 aa, synthetic construct
  REGION 1..37: note = Modified SEQ ID NO 1
  SITE 4: note = MISC_FEATURE - Xaa is norleucine
  SITE 28: note = MISC_FEATURE - Xaa is norleucine
PIEXRCSASVECKQKCLKAIGSIFGKCXNKKCKCYPR

SEQ ID NO: 97 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
PXEXRCSASVECKQKCLAAIGSIFGKCXNKKCKCYPR

SEQ ID NO: 98 - protein, 37 aa, synthetic construct; identical to SEQ ID NO: 97
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine

SEQ ID NO: 99 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
PXEXRCSASVECKQKCLLAIGSIFGKCXNKKCKCYPR

SEQ ID NO: 100 - protein, 37 aa, synthetic construct
  REGION 1..37: note = Modified SEQ ID NO 1
  SITE 28: note = MISC_FEATURE - Xaa is norleucine
PIDERCSASVECKQKCLAAIGSIFGKCXNKKCKCYPR

SEQ ID NO: 101 - protein, 37 aa, synthetic construct
  REGION 1..37: note = Modified SEQ ID NO 1
  SITE 4: note = MISC_FEATURE - Xaa is norleucine
  SITE 28: note = MISC_FEATURE - Xaa is norleucine
PIEXRCSASVECKQKCLAAIGSIFGKCXNKKCKCYPR

SEQ ID NO: 102 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
PXDXRCSASVECAQKCLAAIGSIFGKCMNKKCKCYPR

SEQ ID NO: 103 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
PXEXRCSASVECAQKCLAAIGSIFGKCXNKKCKCYPR

SEQ ID NO: 104 - protein, 32 aa, synthetic construct
  REGION 1..32: note = Modified SEQ ID NO 1
  SITE 23: note = MISC_FEATURE - Xaa is norleucine
CSASVECKQKCLKAIGSIFGKCXNKKCKCYPR

SEQ ID NO: 105 - protein, 32 aa, synthetic construct; identical to SEQ ID NO: 104
  REGION 1..32: note = Modified SEQ ID NO 1
  MOD_RES 1: note = ACETYLATION
  SITE 23: note = MISC_FEATURE - Xaa is norleucine

SEQ ID NO: 106 - protein, 33 aa, synthetic construct
  REGION 1..33: note = Modified SEQ ID NO 1
  SITE 24: note = MISC_FEATURE - Xaa is norleucine
RCSASVECKQKCLKAIGSIFGKCXNKKCKCYPR

SEQ ID NO: 107 - protein, 34 aa, synthetic construct
  REGION 1..34: note = Modified SEQ ID NO 1
  MOD_RES 1: note = ACETYLATION
  SITE 25: note = MISC_FEATURE - Xaa is norleucine
SKCSASVECKQKCLKAIGSIFGKCXNKKCKCYPR

SEQ ID NO: 108 - protein, 34 aa, synthetic construct
  REGION 1..34: note = Modified SEQ ID NO 1
  MOD_RES 1: note = ACETYLATION
  SITE 25: note = MISC_FEATURE - Xaa is norleucine
LRCSASVECKQKCLKAIGSIFGKCXNKKCKCYPR

SEQ ID NO: 109 - protein, 32 aa, synthetic construct
  REGION 1..32: note = Modified SEQ ID NO 1
  MOD_RES 1: note = ACETYLATION
  SITE 23: note = MISC_FEATURE - Xaa is norleucine
CSASVECKQKCLKAIGSIFGKCXNKKCKCYPS

SEQ ID NO: 110 - protein, 31 aa, synthetic construct
  REGION 1..31: note = Modified SEQ ID NO 1
  MOD_RES 1: note = ACETYLATION
  SITE 23: note = MISC_FEATURE - Xaa is norleucine
CSASVECKQKCLKAIGSIFGKCXNKKCKCYS

SEQ ID NO: 111 - protein, 31 aa, synthetic construct
  REGION 1..31: note = Modified SEQ ID NO 1
  MOD_RES 1: note = ACETYLATION
  SITE 23: note = MISC_FEATURE - Xaa is norleucine
CSASVECKQKCLKAIGSIFGKCXNKKCKCYG

SEQ ID NO: 112 - protein, 30 aa, synthetic construct
  REGION 1..30: note = Modified SEQ ID No 1
  MOD_RES 1: note = ACETYLATION
  SITE 23: note = MISC_FEATURE - Xaa is norleucine
CSASVECKQKCLKAIGSIFGKCXNKKCKCY

SEQ ID NO: 113 - protein, 33 aa, synthetic construct; identical to SEQ ID NO: 106
  REGION 1..33: note = Modified SEQ ID NO 1
  MOD_RES 1: note = ACETYLATION
  SITE 24: note = MISC_FEATURE - Xaa is norleucine

SEQ ID NO: 114 - protein, 34 aa, synthetic construct; identical to SEQ ID NO: 108
  REGION 1..34: note = Modified SEQ ID NO 1
  SITE 25: note = MISC_FEATURE - Xaa is norleucine

SEQ ID NO: 115 - protein, 34 aa, synthetic construct
  REGION 1..34: note = Modified SEQ ID NO 1
  MOD_RES 1: note = ACETYLATION
  SITE 25: note = MISC_FEATURE - Xaa is norleucine
LRCSASVECKQKCLAAIGSIFGKCXNKKCKCYPR

SEQ ID NO: 116 - protein, 34 aa, synthetic construct
  REGION 1..34: note = Modified SEQ ID NO 1
  MOD_RES 1: note = ACETYLATION
LRCSASVECKQKCLAAIGSIFGKCMNKKCKCYPR

SEQ ID NO: 117 - protein, 32 aa, synthetic construct
  REGION 1..32: note = Modified SEQ ID NO 1
CSASVECKQKCLKAIGSIFGKCMNKKCKCYPR

SEQ ID NO: 118 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
PXEXRCSASVECKEKCLAAIGSIFGKCXNKKCKCYPR

SEQ ID NO: 119 - protein, 37 aa, synthetic construct; identical to SEQ ID NO: 97
  REGION 1..37: note = Modified SEQ ID NO 1
  SITE 1: note = MISC_FEATURE - D-proline
  SITE 2: note = MISC_FEATURE - Xaa is norleucine
  SITE 4: note = MISC_FEATURE - Xaa is norleucine
  SITE 28: note = MISC_FEATURE - Xaa is norleucine

SEQ ID NO: 120 - protein, 36 aa, synthetic construct
  REGION 1..36: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
PXEXRCSASVECKQKCLAAIGSIFGKCXNKKCKCYR

SEQ ID NO: 121 - protein, 36 aa, synthetic construct
  REGION 1..36: note = Modified SEQ ID NO 1
  SITE 1: note = MISC_FEATURE - Xaa is norleucine
  SITE 3: note = MISC_FEATURE - Xaa is norleucine
  SITE 27: note = MISC_FEATURE - Xaa is norleucine
XEXRCSASVECKQKCLAAIGSIFGKCXNKKCKCYPR

SEQ ID NO: 122 - protein, 38 aa, synthetic construct
  REGION 1..38: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
PXEXRCSASVECKQKCLAAIGSIFGKCXNKKCKCYPRR

SEQ ID NO: 123 - protein, 38 aa, synthetic construct
  REGION 1..38: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
PXEXRCSASVECKQKCLAAIGSIFGKCXNKKCKCYPRY

SEQ ID NO: 124 - protein, 38 aa, synthetic construct
  REGION 1..38: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
PXEXRCSASVECKQKCLAAIGSIFGKCXNKKCKCYPRL

SEQ ID NO: 125 - protein, 38 aa, synthetic construct
  REGION 1..38: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
PXEXRCSASVECKQKCLAAIGSIFGKCXNKKCKCYPRH

SEQ ID NO: 126 - protein, 38 aa, synthetic construct
  REGION 1..38: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
PXEXRCSASVECKQKCLAAIGSIFGKCXNKKCKCYPRE

SEQ ID NO: 127 - protein, 39 aa, synthetic construct
  REGION 1..39: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
PXEXRCSASVECKQKCLAAIGSIFGKCXNKKCKCYPRKS

SEQ ID NO: 128 - protein, 39 aa, synthetic construct
  REGION 1..39: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
PXEXRCSASVECKQKCLAAIGSIFGKCXNKKCKCYPRFE

SEQ ID NO: 129 - protein, 39 aa, synthetic construct
  REGION 1..39: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
PXEXRCSASVECKQKCLAAIGSIFGKCXNKKCKCYPRHR

SEQ ID NO: 130 - protein, 39 aa, synthetic construct
  REGION 1..39: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
PXEXRCSASVECKQKCLAAIGSIFGKCXNKKCKCYPRAK

SEQ ID NO: 131 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
  SITE 37: note = MISC_FEATURE - Xaa is (4-amino-5-hydroxypentyl)guanidine
PXEXRCSASVECKQKCLAAIGSIFGKCXNKKCKCYPX

SEQ ID NO: 132 - protein, 38 aa, synthetic construct
  REGION 1..38: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
  SITE 38: note = MISC_FEATURE - Xaa is 4-amino-5-hydroxypentanamide
PXEXRCSASVECKQKCLAAIGSIFGKCXNKKCKCYPRX

SEQ ID NO: 133 - protein, 39 aa, synthetic construct
  REGION 1..39: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
PXEXRCSASVECKQKCLAAIGSIFGKCXNKKCKCYPRST

SEQ ID NO: 134 - protein, 39 aa, synthetic construct
  REGION 1..39: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
PXEXRCSASVECKQKCLAAIGSIFGKCXNKKCKCYPRRY

SEQ ID NO: 135 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
PXEXRCSSSVECKQKCLAAIGSIFGKCXNKKCKCYPR

SEQ ID NO: 136 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
PXEXRCSLSVECKQKCLAAIGSIFGKCXNKKCKCYPR

SEQ ID NO: 137 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
PXEXRCSAPVECKQKCLAAIGSIFGKCXNKKCKCYPR

SEQ ID NO: 138 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
PXEXRCSASPECKQKCLAAIGSIFGKCXNKKCKCYPR

SEQ ID NO: 139 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
PXEXRCSASQECKQKCLAAIGSIFGKCXNKKCKCYPR

SEQ ID NO: 140 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
PXEXRCSASVECLQKCLAAIGSIFGKCXNKKCKCYPR

SEQ ID NO: 141 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
PXEXRCSASVECKQPCLAAIGSIFGKCXNKKCKCYPR

SEQ ID NO: 142 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
PXEXRCEASVECKQKCLAAIGSIFGKCXNKKCKCYPR

SEQ ID NO: 143 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
PXEXRCFASVECKQKCLAAIGSIFGKCXNKKCKCYPR

SEQ ID NO: 144 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
PXEXRCSYSVECKQKCLAAIGSIFGKCXNKKCKCYPR

SEQ ID NO: 145 - protein, 37 aa, synthetic construct
  REGION 1..37: note = Modified SEQ ID NO 1
  SITE 2: note = MISC_FEATURE - Xaa is norleucine
  SITE 4: note = MISC_FEATURE - Xaa is norleucine
  SITE 28: note = MISC_FEATURE - Xaa is norleucine
PXEXRCSAFVECKQKCLAAIGSIFGKCXNKKCKCYPR

SEQ ID NO: 146 - protein, 4 aa, synthetic construct
  REGION 1..4: note = Additional C-terminal sequence
RRTA

SEQ ID NO: 147 - protein, 4 aa, synthetic construct
  REGION 1..4: note = Additional C-terminal sequence
HRRK

SEQ ID NO: 148 - protein, 4 aa, synthetic construct
  REGION 1..4: note = Additional C-terminal sequence
QSKA

SEQ ID NO: 149 - protein, 4 aa, synthetic construct
  REGION 1..4: note = Additional C-terminal sequence
AGPR

SEQ ID NO: 150 - protein, 4 aa, synthetic construct
  REGION 1..4: note = Additional C-terminal sequence
RSRT

SEQ ID NO: 151 - protein, 4 aa, synthetic construct
  REGION 1..4: note = Additional C-terminal sequence
RHKR

SEQ ID NO: 152 - protein, 4 aa, synthetic construct
  REGION 1..4: note = Additional C-terminal sequence
GGKR

SEQ ID NO: 153 - protein, 4 aa, synthetic construct
  REGION 1..4: note = Additional C-terminal sequence
PKTA

SEQ ID NO: 154 - protein, 4 aa, synthetic construct
  REGION 1..4: note = Additional C-terminal sequence
TDAR

SEQ ID NO: 155 - protein, 4 aa, synthetic construct
  REGION 1..4: note = Additional C-terminal sequence
HRQQ

SEQ ID NO: 156 - protein, 4 aa, synthetic construct
  REGION 1..4: note = Additional C-terminal sequence
RPRH

SEQ ID NO: 157 - protein, 4 aa, synthetic construct
  REGION 1..4: note = Additional C-terminal sequence
ARNA

SEQ ID NO: 158 - protein, 4 aa, synthetic construct
  REGION 1..4: note = Additional C-terminal sequence
TGRK

SEQ ID NO: 159 - protein, 4 aa, synthetic construct
  REGION 1..4: note = Additional C-terminal sequence
HERT

SEQ ID NO: 160 - protein, 4 aa, synthetic construct
  REGION 1..4: note = Additional C-terminal sequence
NTRT

SEQ ID NO: 161 - protein, 4 aa, synthetic construct
  REGION 1..4: note = Additional C-terminal sequence
QRNG

SEQ ID NO: 162 - protein, 4 aa, synthetic construct
  REGION 1..4: note = Additional C-terminal sequence
AHRN

SEQ ID NO: 163 - protein, 4 aa, synthetic construct
  REGION 1..4: note = Additional C-terminal sequence
PRSA

SEQ ID NO: 164 - protein, 4 aa, synthetic construct
  REGION 1..4: note = Additional C-terminal sequence
QRQS

SEQ ID NO: 165 - protein, 4 aa, synthetic construct
  REGION 1..4: note = Additional C-terminal sequence
QRRK

SEQ ID NO: 166 - protein, 4 aa, synthetic construct
  REGION 1..4: note = Additional C-terminal sequence
ARAK

SEQ ID NO: 167 - protein, 4 aa, synthetic construct
  REGION 1..4: note = Additional C-terminal sequence
AKRD

SEQ ID NO: 168 - protein, 4 aa, synthetic construct
  REGION 1..4: note = Additional C-terminal sequence
RDKT

SEQ ID NO: 169 - protein, 4 aa, synthetic construct
  REGION 1..4: note = Additional C-terminal sequence
HRRK

SEQ ID NO: 170 - protein, 4 aa, synthetic construct
  REGION 1..4: note = Additional C-terminal sequence
RAKR

SEQ ID NO: 171 - protein, 4 aa, synthetic construct
  REGION 1..4: note = Additional C-terminal sequence
QRTR

SEQ ID NO: 172 - protein, 4 aa, synthetic construct
  REGION 1..4: note = Additional C-terminal sequence
ATRH

SEQ ID NO: 173 - protein, 4 aa, synthetic construct
  REGION 1..4: note = Additional C-terminal sequence
ARRS

SEQ ID NO: 174 - protein, 4 aa, synthetic construct
  REGION 1..4: note = Additional C-terminal sequence
AKTR

SEQ ID NO: 175 - protein, 4 aa, synthetic construct
  REGION 1..4: note = Additional C-terminal sequence
NRQR

SEQ ID NO: 176 - protein, 4 aa, synthetic construct
  REGION 1..4: note = Additional C-terminal sequence
PRNT

SEQ ID NO: 177 - protein, 35 aa, Stichodactyla helianthus
RSCIDTIPKSRCTAFQCKHSMKYRLSFCRKTCGTC

SEQ ID NO: 178 - protein, 37 aa, synthetic construct
  REGION 1..37: note = Modified SEQ ID NO 177 (ShK-186)
  SITE 1: note = MISC_FEATURE - Xaa is phosphotyrosyl
  SITE 2: note = MISC_FEATURE - Xaa is 8-amino-3,6-dioxaoctanoyl
XXRSCIDTIPKSRCTAFQCKHSMKYRLSFCRKTCGTC

SEQ ID NO: 179 - protein, 34 aa, synthetic construct
  REGION 1..34: note = Mokatoxin1 (Moka1)
INVKCSLPQQCIKPCKDAGMRFGKCMNKKCRCYS

SEQ ID NO: 180 - protein, 36 aa, unidentified
  REGION 1..36: note = Vaejovis mexicanus toxin (Vm24)
AAAISCVGSPECPPKCRAQGCKNGKCMNRKCKCYYC

(19) United States
(12) Patent Application Publication
(10) Pub. No.: US 2024/0076328 A1
(43) Pub. Date: Mar. 7, 2024

(54) KV1.3 BLOCKERS

(71) Applicant: Zealand Pharma A/S, Søborg (DK)

(72) Inventors: Henrik Fischer Munch, Søborg (DK); Rasmus Bugge Jensen, Søborg (DK); Jens Kvist Madsen, Søborg (DK)

(21) Appl. No.: 18/352,906

(22) Filed: Jul. 14, 2023

Related U.S. Application Data
(63) Continuation of application No. 17/679,925, filed on Feb. 24, 2022, now Pat. No. 11,780,893.
Application No. 17/679,925 is a continuation of application No. 17/356,354, filed on Jun. 23, 2021, now Pat. No. 11,292,820.
Application No. 17/356,354 is a continuation of application No. PCT/EP2020/076187, filed on Sep. 18, 2020.

(30) Foreign Application Priority Data
Sep. 20, 2019 (EP) 19198763.5
May 5, 2020 (EP) 20172989.4

(51) Int. Cl. C07K 14/435

(52) U.S. Cl. CPC C07K 14/43522; A61K 38/00

(57) ABSTRACT

The present invention provides novel blockers of the potassium channel Kv1.3, polynucleotides encoding them, and methods of making and using them.

### CROSS-REFERENCE TO RELATED APPLICATIONS

This application is a Continuation of U.S. application Ser. No. 17/679,925, filed Feb. 24, 2022, which is a Continuation of U.S. application Ser. No. 17/356,354, filed Jun. 23, 2021, which is a Continuation of International Application No. PCT/EP2020/076187, filed Sep. 18, 2020, which claims priority to European Application No. 19198763.5, filed Sep. 20, 2019 and European Application No. 20172989.4, filed May 5, 2020.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “56632C_Seqlisting.txt”, which was created on Jun. 29, 2023 and is 326,189 bytes in size. The subject matter of the Sequence Listing is incorporated herein in its entirety by reference.

### FIELD OF THE INVENTION

The present invention provides novel blockers of the potassium channel Kv1.3, polynucleotides encoding them, and methods of making and using them.

### BACKGROUND OF THE INVENTION

Ion channels are membrane proteins which form pores in biological membranes, permitting (and regulating) the flow of ions across the relevant membrane. There are numerous different types of ion channel, which may be classified in various ways, such as by the species of ions to which they provide passage, the way in which passage of ions is regulated or “gated” (e.g. “ligand-gated” or “voltage-gated”), and their cellular or sub-cellular localisation.

Potassium channels fall into four major classes, namely voltage-gated potassium channels, calcium-activated potassium channels, inwardly rectifying potassium channels, and tandem pore domain potassium channels.

The voltage-gated potassium channels, like other voltage gated channels, open or close in response to transmembrane voltages. They represent a complex family with diverse biological functions, including the regulation of neurotransmitter release, heart rate, insulin secretion, neuronal excitability, epithelial electrolyte transport, smooth muscle contraction, and cell volume.

The Kv1.3 (potassium voltage-gated channel subfamily A member 3) channel is expressed on T cells and plays a role in regulating T cell activation. Blockers of Kv1.3 have been shown to inhibit proliferation of activated T cells in vitro (reviewed in Cahalan and Chandy, Immunol. Rev. 231:59-87, 2009), and to inhibit T cell-dependent disease progression in various experimental models of autoimmune disease including experimental autoimmune encephalomyelitis (EAE), experimental arthritis, delayed-type hypersensitivity (DTH), allergic contact dermatitis and glomerulonephritis. See, for example, Rangaraju et al. (Expert Opin. Ther. Targets 13:909-24, 2009); Beeton et al. (Proc. Natl. Acad. Sci. USA. 103:17414-9, 2006); Koo et al. (J. Immunol. 158:5120-8, 1997); Hyodo et al. (Am. J. Physiol. Renal Physiol. 299: F1258-69, 2010). WO 2016/112208 describes topical application of Kv1.3 blockers for the treatment of skin and mucosal inflammation.

Thus, Kv1.3 blockers have considerable potential for use in treatment of inflammatory disorders, including autoimmune disorders.

WO 2015/013330 proposes use of Kv1.3 blocker peptides for treatment of ophthalmic conditions, such as dry eye and uveitis, including when caused by autoimmune conditions such as Sjogren's syndrome.

Blockers of Kv1.3 may also have beneficial metabolic effects, e.g. in relation to energy homeostasis, body weight regulation, and glucose control. Kv1.3 knock-out (Kv1.3(−/−)) mice exhibit reduced weight gain, higher insulin sensitivity, and reduced plasma glucose levels in response to a high fat diet as compared to control littermates (Xu et al., Hum. Mol. Genet. 12:551-9, 2003). Further, Kv1.3 blockers have been shown to increase expression in skeletal muscle and adipose tissue of glucose transporter 4 (GLUT4), to increase insulin sensitivity in normal and ob/ob obese mice, and to increase glucose uptake in primary adipocytes in vitro (Xu et al., Proc. Natl. Acad. Sci. USA 101:3112-7, 2004). In humans, a single nucleotide polymorphism (SNP) in the Kv1.3 gene has also been associated with decreased insulin sensitivity and impaired glucose tolerance (Tschritter, Clin Endocrinol Metab 91: 654-8, 2006).

Kv1.3 is also expressed in proliferating human and mouse smooth muscle cells. Blockers of Kv1.3 may be effective in smooth muscle proliferative disorders such as restenosis, e.g. in patients following vascular surgery (e.g. angioplasty). Kv1.3 blockers have been shown to inhibit calcium entry, reduce smooth muscle cell migration, and inhibit neointimal hyperplasia in ex vivo human vein samples (Cheong et al., Cardiovasc. Res. 89:282-9, 2011).

Further evidence suggests that Kv1.3 channels are involved in the activation and/or proliferation of many types of cells, including tumor cells (Bielanska et al., Curr. Cancer Drug Targets 9:904-14, 2009), microglia (Khanna et al., Am. J. Physiol. Cell Physiol. 280: C796-806, 2001) and differentiation of neuronal progenitor cells (Wang et al., J. Neurosci. 30:5020-7, 2010). Kv1.3 blockers may therefore be beneficial in the treatment of neuroinflammatory and neurodegenerative disorders, and cancers.

Kv1.3 is part of a sub-family of closely related potassium channels, designated Kv1.1 to Kv1.8. When dealing with large homologous families, it is always desirable for a blocker to be as selective and specific as possible for the desired target, to improve efficacy and safety, and avoid undesirable off-target effects. The most specific Kv1.3 blockers identified to date are venom peptides derived from various types of venomous organisms, such as snakes, arachnids (such as scorpions and spiders), sea anemones, etc. Such Kv1.3 blockers include the peptides ShK, Oskl, margatoxin and kaliotoxin, reviewed by Chandy et al., Trends in Pharmacol. Sci. 25:280-9, 2004. See also Abdel-Mottaleb et al., Toxicon 51:1424-30, 2008, and Mouhat et al., Biochem. J. 385(Pt 1):95-104, 2005.

Various attempts to engineer toxin peptides for particular properties, including specificity or potency, have been described, e.g. in WO2006/002850, WO2006/042151, WO2008/088422, WO2006/116156, WO2010/105184 and WO2014/116937.

However, there remains a need for alternative Kv1.3 blockers. Blockers having improved specificity compared to known blockers may be particularly desirable, although improvements in other properties such as stability and potency may also be useful.

### SUMMARY OF THE INVENTION

The invention relates to ion channel blockers from the scorpion Parabuthus transvaalicus, having the amino acid sequences:

(PaT1)
QMDMRCSASVECKQKCLKAIGSIFGKCMNKKCKCYPR;
and
(PaT2)
QMDMRCSASVECKQKCLKAIGRGFGKCMNKKCKCYPR;

and variants thereof.

Amongst other desirable properties, these molecules have been found to be extremely selective blockers for Kv1.3 over other voltage-gated potassium channels, and typically also have high potency at the Kv1.3 channel.

The invention therefore provides an ion-channel blocker comprising a Kv1.3 inhibitor component, said Kv1.3 inhibitor component comprising:
the sequence of PaT1:

QMDMRCSASVECKQKCLKAIGSIFGKCMNKKCKCYPR;

or a variant thereof which differs from PaT1 by up to 9 substitutions, insertions or deletions, wherein any substitutions or deletions are at amino acid positions selected from positions 1-5, 7-11, 13-15, 17-23, 25, 28-31, 33 and 35-37; and
wherein said Kv1.3 inhibitor component has Kv1.3 inhibitor activity and is selective for Kv1.3;
or a pharmaceutically acceptable salt thereof.

Thus, the Kv1.3 inhibitor component of the ion channel blocker can contain up to 9 amino acid changes compared to the sequence of PaT1. Except where otherwise specified, each those 9 changes can independently be selected from a single amino acid insertion, deletion or substitution. In an embodiment, the Kv1.3 inhibitor component of the ion channel blocker may contain 9, 8, 7, 6, 5, 4, 3, 2 or 1 amino acid changes compared to the sequence of PaT1. In an embodiment, the Kv1.3 inhibitor component of the ion channel blocker contains 6 amino acid changes compared to the sequence of PaT1.

The amino acid residues of PaT1 are numbered from 1 to 33, in the conventional direction of N- to C-terminus. Throughout this specification, amino acid positions in variants of PaT1 are numbered according to the corresponding position in PaT1 when optimally aligned therewith. Thus, especially for inhibitors which contain one or more insertions or deletions compared to PaT1, the numbering of any given residue reflects the corresponding residue in PaT1 and does not necessarily reflect its linear position in the relevant sequence.

The residue present at a specific position may be indicated by the number of the relevant position alongside the single letter code or three letter code for the residue present. Thus, 1Q or Q1 (the two formats are interchangeable) indicates a glutamine (Q) residue at position 1, while 2Nle, 2[Nle], Nle2 or [Nle]2 indicates a norleucine residue at position 2.

An asterisk may be used to denote the position of a deletion relative to the sequence of PaT1. For example, “1*” indicates a deletion of the residue at position 1 as compared to PaT1.

An insertion may be indicated by a string of consecutive residues at a single position, e.g. “1QA” indicates an insertion of an alanine (A) residue after the glutamine (Q) residue at position 1.

In some embodiments, it may be desirable that one or more of positions 28, 31, 34, 35, 36 or 37 are identical to the corresponding position in PaT1, i.e. the residue present in the relevant position(s) is identical to the residue present in PaT1 at the corresponding position. For example, one or more of positions 28, 31, 34 and 35 may be identical to the corresponding position in PaT1. In some embodiments, all of positions 28, 31, 34 and 35 are identical to the corresponding positions in PaT1, e.g. all of positions 28, 31, 34, 35, 36 and 37 are identical to the corresponding positions in PaT1.

It may be desirable that any substitutions compared to PaT1 are conservative substitutions. However, any substitution listed in any of the generic formulae provided below may be introduced at the respective position.

The Kv1.3 inhibitor component of the molecule may comprise the sequence of PaT1:

QMDMRCSASVECKQKCLKAIGSIFGKCMNKKCKCYPR

or may differ from PaT1 by up to 9 substitutions, insertions or deletions, wherein any substitutions or deletions are made only at the following positions wherein, if different from PaT1:
the residue at position 1 is selected from N, P, I, V, H, Y and S or is deleted;
the residue at position 2 is selected from Nle, I, V, L, T, Q and E or is deleted;
the residue at position 3 is selected from E, S and N or is deleted;
the residue at position 4 is selected from Nle, I, V, L, A, E and S or is deleted;
the residue at position 5 is selected from K, Q, A, L, E and D or is deleted;
the residue at position 7 is selected from K, R, Q, E, F and A;
the residue at position 8 is selected from I, H, S, L, Y and G;
the residue at position 9 is selected from A, Abu, P, F, V and L;
the residue at position 10 is selected from R, K, P, A, Q and L;
the residue at position 11 is selected from Q and D;
the residue at position 13 is selected from L, A, E, Q, I, K, H, D, V and G;
the residue at position 14 is selected from V, K, E, L, D and 2-amino-5-carboxypentanoyl;
the residue at position 15 is selected from S, L and P;
the residue at position 17 is selected from K, Y, R, Q, D, V and E or is deleted;
the residue at position 18 is selected from D, A, Y, G, V, Q, hQ and L or is deleted;
the residue at position 19 is selected from R, Y and Q or is deleted;
the residue at position 20 is selected from Y, E and R or is deleted;
the residue at position 21 is selected from R, H, E and D;
the residue at position 22 is selected from R, M, D, L and C;
the residue at position 23 is selected from R, K, hK, P, G and H;
the residue at position 26 is homo-lysine;
the residue at position 28 is selected from Nle, A and L;
the residue at position 29 is V;
the residue at position 30 is selected from G and D;
the residue at position 31 is selected from D, Q, E and H;
the residue at position 33 is selected from H, V, D, Q and R;
the residue at position 35 is selected from T, F(4-NH₂), F(4-F), F(4-NO₂) and F(4-CH₃);
the residue at position 36 is selected from Q, S and G or is deleted;
the residue at position 37 is selected from K, E, S and C or is deleted.

The Kv1.3 inhibitor component of the molecule may comprise the sequence of PaT1:

QMDMRCSASVECKQKCLKAIGSIFGKCMNKKCKCYPR

or may differ from PaT1 by up to 9 substitutions, insertions or deletions, wherein any substitutions or deletions are made only at the following positions wherein, if different from PaT1:
the residue at position 1 is selected from N, P, I, V, H, Y and S or is deleted;
the residue at position 2 is selected from Nle, I, V, L, T, Q and E or is deleted;
the residue at position 3 is selected from E, S and N or is deleted;
the residue at position 4 is selected from Nle, I, V, L, A, E and S or is deleted;
the residue at position 5 is selected from K, Q, A, L, E and D or is deleted;
the residue at position 7 is selected from K, R, Q and A;
the residue at position 8 is selected from I, H and G;
the residue at position 9 is selected from A, Abu and L;
the residue at position 10 is selected from R, K, P, A and L;
the residue at position 11 is selected from Q and D;
the residue at position 13 is selected from L, A, E, Q, I, K, H, D, V and G;
the residue at position 14 is selected from V, K, E, L, D and 2-amino-5-carboxypentanoyl;
the residue at position 15 is selected from S, L and P;
the residue at position 17 is selected from K, Y, R, Q, D, V and E or is deleted;
the residue at position 18 is selected from D, A, Y, G, V, Q, hQ and L or is deleted;
the residue at position 19 is selected from R, Y and Q or is deleted;
the residue at position 20 is selected from Y, E and R or is deleted;
the residue at position 21 is selected from R, H, E and D;
the residue at position 22 is selected from R, M, D, L and C;
the residue at position 23 is selected from R, K, hK, P, G and H;
the residue at position 28 is selected from Nle, A and L;
the residue at position 29 is V;
the residue at position 30 is selected from G and D;
the residue at position 31 is selected from D, Q, E and H;
the residue at position 33 is selected from H, V, D, Q and R;
the residue at position 35 is selected from T, F(4-NH₂), F(4-F), F(4-NO₂) and F(4-CH₃);
the residue at position 36 is selected from Q, S and G or is deleted;
the residue at position 37 is selected from K, E, S and C or is deleted.

In some embodiments, the Kv1.3 inhibitor component may differ from PaT1 by up to 9 substitutions, insertions or deletions, wherein any substitutions or deletions are made only at the following positions wherein, if different from PaT1:
the residue at position 1 is selected from N, P, I, V, H, Y and S or is deleted;
the residue at position 2 is selected from Nle, I, V, L, T, Q and E or is deleted;
the residue at position 3 is selected from E, S and N or is deleted;
the residue at position 4 is selected from Nle, I, V, L, A, E and S or is deleted;
the residue at position 5 is selected from K, Q, A and L or is deleted;
the residue at position 7 is selected from K, R and Q;
the residue at position 8 is selected from I, H, S, L, Y and G;
the residue at position 9 is selected from A, Abu, P, F, V and L;
the residue at position 10 is selected from R, K and P;
the residue at position 11 is Q;
the residue at position 13 is selected from L, A, E, Q, I, K, H and G;
the residue at position 14 is selected from V, K, E, L and 2-amino-5-carboxypentanoyl;
the residue at position 15 is selected from S and L;
the residue at position 17 is selected from K, Y, R, Q and D or is deleted;
the residue at position 18 is selected from D, A, Y, G, V, Q, hQ and L or is deleted;
the residue at position 19 is selected from R, Y and Q or is deleted;
the residue at position 20 is selected from Y, E and R or is deleted;
the residue at position 21 is selected from R, H and E;
the residue at position 22 is selected from R, M, D, L and C;
the residue at position 23 is selected from R, K, hK, P and G;
the residue at position 28 is Nle;
the residue at position 30 is selected from G and D;
the residue at position 31 is selected from D, Q, E and H;
the residue at position 33 is selected from H, V, D, Q and R;
the residue at position 35 is selected from T, F(4-NH₂), F(4-F), F(4-NO₂) and F(4-CH₃);
the residue at position 36 is selected from Q, S and G or is deleted;
the residue at position 37 is selected from K, S and C or is deleted.

In some embodiments, the Kv1.3 inhibitor component may differ from PaT1 by up to 9 substitutions, insertions or deletions, wherein any substitutions or deletions are made only at the following positions wherein, if different from PaT1:
the residue at position 1 is selected from N, P, I, V, H, Y and S or is deleted;
the residue at position 2 is selected from Nle, I, V, L, T, Q and E or is deleted;
the residue at position 3 is selected from E, S and N or is deleted;
the residue at position 4 is selected from Nle, I, V, L, A, E and S or is deleted;
the residue at position 5 is selected from K, Q, A and L or is deleted;
the residue at position 7 is selected from K, R and Q;
the residue at position 8 is selected from I, H and G;
the residue at position 9 is selected from A, Abu and L;
the residue at position 10 is selected from R, K and P;
the residue at position 11 is Q;
the residue at position 13 is selected from L, A, E, Q, I, K, H and G;
the residue at position 14 is selected from V, K, E, L and 2-amino-5-carboxypentanoyl;
the residue at position 15 is selected from S and L;
the residue at position 17 is selected from K, Y, R, Q and D or is deleted;
the residue at position 18 is selected from D, A, Y, G, V, Q, hQ and L or is deleted;
the residue at position 19 is selected from R, Y and Q or is deleted;
the residue at position 20 is selected from Y, E and R or is deleted;
the residue at position 21 is selected from R, H and E;
the residue at position 22 is selected from R, M, D, L and C;
the residue at position 23 is selected from R, K, hK, P and G;
the residue at position 28 is Nle;
the residue at position 30 is selected from G and D;
the residue at position 31 is selected from D, Q, E and H;
the residue at position 33 is selected from H, V, D, Q and R;
the residue at position 35 is selected from T, F(4-NH₂), F(4-F), F(4-NO₂) and F(4-CH₃);
the residue at position 36 is selected from Q, S and G or is deleted;
the residue at position 37 is selected from K, S and C or is deleted.

The Kv1.3 inhibitor component of the molecule may comprise the sequence of PaT1:

QMDMRCSASVECKQKCLKAIGSIFGKCMNKKCKCYPR

or may differ from PaT1 by up to 9 substitutions, insertions or deletions, wherein any substitutions or deletions are made only at the following positions wherein, if different from PaT1:
the residue at position 1 is selected from N, P, V, H, Y and S or is deleted;
the residue at position 2 is selected from Nle, I, V and L or is deleted;
the residue at position 3 is selected from E and S or is deleted;
the residue at position 4 is selected from Nle, I, V, L, E and S or is deleted;
the residue at position 5 is K or is deleted;
the residue at position 7 is selected from K and R;
the residue at position 8 is selected from I and H;
the residue at position 9 is selected from Abu and L;
the residue at position 10 is selected from R and K;
the residue at position 11 is Q;
the residue at position 13 is selected from L, A, E, Q, V and G;
the residue at position 14 is selected from V, K, E, L and 2-amino-5-carboxypentanoyl;
the residue at position 15 is selected from S and L;
the residue at position 17 is selected from K, Y and R or is deleted;
the residue at position 18 is selected from D, A, Y, G, V, Q, hQ and L or is deleted;
the residue at position 19 is selected from R and Y or is deleted;
the residue at position 20 is selected from Y, E and R or is deleted;
the residue at position 21 is selected from R, H and E;
the residue at position 22 is selected from R and C;
the residue at position 23 is selected from R, K, hK, P and G;
the residue at position 28 is Nle;
the residue at position 30 is G;
the residue at position 33 is selected from H, V and R;
the residue at position 35 is selected from F(4-NH₂), F(4-F), F(4-NO₂) and F(4-CH₃);
the residue at position 36 is selected from Q, S and G or is deleted;
the residue at position 37 is selected from S and C or is deleted.

In some embodiments, the Kv1.3 inhibitor component of the molecule comprises the sequence of PaT1:

QMDMRCSASVECKQKCLKAIGSIFGKCMNKKCKCYPR

or differs from PaT1 by up to 9 substitutions, insertions or deletions, wherein any substitutions or deletions are made only at the following positions wherein, if different from PaT1:
the residue at position 1 is selected from N and P or is deleted;
the residue at position 2 is Nle, I, V and L;
the residue at position 3 is selected from E and S;
the residue at position 4 is Nle, I, V and L;
the residue at position 8 is I;
the residue at position 10 is R;
the residue at position 11 is Q;
the residue at position 13 is A;
the residue at position 14 is V;
the residue at position 18 is selected from D, Y and A;
the residue at position 19 is R;
the residue at position 20 is Y;
the residue at position 21 is R;
the residue at position 22 is selected from R and C;
the residue at position 23 is selected from R and G;
the residue at position 28 is Nle;
the residue at position 30 is G;
the residue at position 33 is H;
the residue at position 37 is C.

In some embodiments the invention provides an ion-channel blocker comprising a Kv1.3 inhibitor component, said Kv1.3 inhibitor component comprising: the sequence of PaT1:

QMDMRCSASVECKQKCLKAIGSIFGKCMNKKCKCYPR;

or a sequence which has at least 75% identity with the sequence of PaT1, wherein any differences from the sequence of PaT1 are at amino acid positions selected from positions 1-5, 7-11, 13-15, 17-23, 25, 28-31, 33 and 35-37; and
wherein said Kv1.3 inhibitor component has Kv1.3 inhibitor activity and is selective for Kv1.3;
or a pharmaceutically acceptable salt thereof.

In some embodiments the Kv1.3 inhibitor component has at least 75% identity with the sequence of PaT1. In some embodiments the Kv1.3 inhibitor component has at least 80% identity with the sequence of PaT1. In some embodiments the Kv1.3 inhibitor component has at least 85% identity with the sequence of PaT1. In some embodiments the Kv1.3 inhibitor component has at least 90% identity with the sequence of PaT1. In some embodiments the Kv1.3 inhibitor component has at least 95% identity with the sequence of PaT1. In some embodiments the Kv1.3 inhibitor component has at least 97% identity with the sequence of PaT1.

The Kv1.3 inhibitor component may comprise one or more insertions or deletions as compared to the sequence of PaT1.

Typically, the Kv1.3 inhibitor component comprises a maximum of three insertions as compared to the sequence of PaT1, e.g. precisely 3 insertions, precisely 2 insertions, or precisely 1 insertion.

Typically, only a single amino acid is inserted at any given site, although insertions of multiple amino acids may be tolerated. For the avoidance of doubt, an insertion of two amino acids at the same site is considered as two insertions and hence as two of the permitted differences from the sequence of PaT1.

Typically, the Kv1.3 inhibitor component contains no more than 7 deletions compared to the sequence of PaT1, e.g. no more than 6, no more than 5, no more than 5, no more than 4, no more than 3, no more than 2 deletions, no more than one deletion, or no deletions at all.

In order to preserve the spacing of the cysteine residues, and hence the pattern of disulphide bonding, it may be desirable that any insertions and deletions are not located between the cysteine residue at position 6 (6C) and the cysteine residue at position 34 (34C). Thus, any insertion or deletion may preferably be located N-terminal of 6C or C-terminal of 34C.

However, if the Kv1.3 inhibitor component contains one or more insertions or deletions between 6C and 34C, then it may be desirable to incorporate the same number of both insertions and deletions within that region. e.g. to restore the relative spacing of the cysteine residues.

In many embodiments the Kv1.3 inhibitor component has no insertions as compared to the sequence of PaT1.

If a deletion is present between 6C and 34C, it may be desirable that only one such deletion is present. It may, for example, be at position 17, 18, 19 or 20, especially if the molecule does not contain a corresponding insertion to maintain the spacing of the cysteine residues.

Without wishing to be bound by theory, it is believed that multiple deletions may be better tolerated in the N-terminal sequence (residues 1-5) and C-terminal sequence (residues 36-37). Multiple deletions may be tolerated in these regions. For deletions in the N-terminal sequence (positions 1-5), if a residue at position X is deleted, it may be desirable that all residues N-terminal of that residue are also deleted. Thus, for example, residues 1, 1 and 2, 1 to 3, 1 to 4 or 1 to 5 may be deleted. For deletions at the C-terminus (positions 36-37), if the residue at position 36 is deleted, it may be desirable that the residue at position 37 is also deleted. Thus, for example, residues 37, or 36 and 37, may be deleted.

For the avoidance of doubt, a deletion of two consecutive amino acids is considered as two amino acid deletions and hence as two of the permitted differences from the sequence of PaT1.

Glutamine (Q) residues can be unstable, either in vivo or in vitro, e.g. during storage in aqueous solution. This may be of particular relevance when the glutamine residue is located at the N-terminus of the molecule, as the side chain may be sterically capable of interacting with the free alpha amino group, resulting in dehydration to pyroglutamate. Thus it may be desirable that the residue at position 1 is not Q, especially if the ion channel blocker does not contain any additional sequence N-terminal of the Kv1.3 inhibitor. For example, the residue at position 1 may be N or P, or may be deleted; i.e. the Kv1.3 inhibitor may comprise 1N, 1P or 1*.

Additionally or alternatively, it may be desirable that one, two or all three of the residues at positions 2, 4 and 28 are not M, since methionine (M) residues are susceptible to oxidation. Preferably one, two or all three methionine residues are either individually substituted with a residue having a non-oxidisable side chain, or are deleted. Any suitable residue with a non-oxidisable residue may be used, with Nle. I, V and L being particularly suitable.

Suitable combinations of residues at some or all of positions 2, 4 and 28 include:
2*+4Nle, 4I, 4V or 4L;
2Nle+4Nle, 4I, 4V or 4L;
2I+4Nle, 4I, 4V or 4L;
2V+4Nle, 4I, 4V or 4L;
2L+4Nle, 4I, 4V or 4L.

Any of the above may be combined with 28Nle.
2Nle+4Nle+28Nle may be particularly preferred.

In some embodiments, the Kv1.3 inhibitor component contains the residues 22S+23I or 22R+23G.

In addition to any differences at positions 1, 2, 4 and 28, it may be desirable that the Kv1.3 inhibitor component contains only 4 further differences, only 3 further differences, only 2 further differences, or only 1 further difference from the sequence of PaT1.

The Kv1.3 inhibitor component may be identical to PaT1 or PaT2 at all positions apart from positions 1, 2, 4 and 28.

Additionally or alternatively, the Kv1.3 inhibitor component may comprise the residues 13A and/or 18A.

For example, it may comprise
2Nle+13A;
2Nle+18A, or
2Nle+13A+18A.

For example, it may comprise
2Nle+4Nle+13A+28Nle;
2Nle+4Nle+18A+28Nle; or
2Nle+4Nle+13A+18A+28Nle.

Any of the above may be combined with 1*.

The Kv1.3 inhibitor component may be identical to PaT1 or PaT2 at all positions apart from positions 2, 3 and 4.

Additionally or alternatively, the Kv1.3 inhibitor component may comprise the residues 1P and/or 18A.

For example it may comprise
2Nle+4Nle+28Nle;
2Nle+3E+4Nle+28Nle;
2Nle+3E+4Nle+18A+28Nle;
1P+2Nle+4Nle+28Nle;
1P+2Nle+3E+4Nle+28Nle; or
1P+2Nle+3E+4Nle+18A+28Nle.

Additionally or alternatively, any of the above may be combined with 22C+37C, i.e. residues at positions 22 and 37 capable of forming a disulphide bond. The ion channel blocker may comprise additional amino acid sequences N-terminal and/or C-terminal of the Kv1.3 inhibitor component. For example, the ion channel blocker may be a fusion protein comprising the Kv1.3 inhibitor and one or more heterologous peptide or polypeptide sequences, which may be referred to as heterologous components. The heterologous components may serve, for example, to facilitate recombinant expression, increase solubility or extend half life in vivo. Suitable heterologous components include human serum albumin (HSA), an antibody Fc domain, etc.

Other heterologous components include tags such as a polyhistidine tag, FLAG tag or Myc tag.

The ion channel blocker may comprise a linker peptide located between the Kv1.3 inhibitor and the heterologous component.

The various components may be in any appropriate orientation. For example, the Kv1.3 inhibitor may be located N-terminal of the heterologous component, or the heterologous component may be located N-terminal of the Kv1.3 inhibitor, with the linker (where present) between them.

A peptide linker is typically between 3 and 30 amino acids in length, with a high proportion of small and hydrophilic amino acid residues (e.g. glycine and serine) to provide the required flexibility without compromising aqueous solubility of the molecule. For example, it may comprise at least 50% glycine and serine residues, at least 60% glycine and serine residues, at least 70% glycine and serine residues, at least 80% glycine and serine residues, or at least 90% glycine and serine residues. It may also contain a protease cleavage site to enable separation of the Kv1.3 inhibitor and heterologous component.

The Kv1.3 inhibitor may also be inserted within a heterologous polypeptide, which may be regarded as a “scaffold” for the Kv1.3 inhibitor. In such cases, the ion channel blocker may be considered to comprise heterologous components N- and C-terminal of the Kv1.3 inhibitor, wherein the heterologous components are derived from the same molecule and interact with one another, e.g. to fold into a scaffold, having the Kv1.3 inhibitor displayed at its surface. Thus, the Kv1.3 inhibitor may be inserted within a surface loop of a heterologous protein, e.g. into the CDR sequence of an antibody or a fragment thereof containing the antigen binding domain. It has been demonstrated that a Kv1.3 inhibitor retains activity when inserted into a CDR (e.g. CDR3L) of a humanised immunoglobulin designated “Syn”, directed against respiratory syncytial virus. (Wang et al., Proc. Natl. Acad. Sci. USA 113(41), 11501-11506, 2016.) The Syn antibody, or a fragment thereof containing the antigen binding domain, may therefore be used as a scaffold. If the antibody scaffold contains an Fc domain, it may be desirable that this is functionally “Fc-null”, i.e. it is not capable of binding to the Fc receptor. Such mutations are described by Wang et al., op cit.

In some embodiments, the ion channel blocker may have a maximum length of 200 amino acids, e.g. of 150 amino acids, 125 amino acids, 100 amino acids, 75 amino acids or 50 amino acids, e.g. 49, 48, 47, 46, 45, 44, 43, 42, 41, 40, 39, 38 or 37 amino acids, or even shorter where the Kv1.3 inhibitor component contains one or more deletions compared to the sequence of PaT1.

The ion channel blocker may further comprise up to 10 additional residues at the N-terminus, e.g. 1, 2, 3, 4, 5, 6, 7, 8, 9 or 10 additional residues at the N-terminus. Additionally or alternatively, the ion channel blocker may further comprise up to 10 residues at the C-terminus, e.g. 1, 2, 3, 4, 5, 6, 7, 8, 9 or 10 additional residues at the C-terminus, within the constraint that the molecule is not more than 50 amino acids in length.

For example, the additional sequence at the N-terminus may comprise or consist of the sequence GG or SG. Additionally or alternatively, the additional sequence at the C-terminus may comprise or consist of the sequence RRTA, HRRK, QSKA, AGPR, RSRT, RHKR, GGKR, PKTA, TDAR, HRQQ, RPRH, ARNA, TGRK, HERT, NTRT, QRNG, AHRN, PRSA, QRQS, QRRK, ARAK, AKRD, RDKT, HRRK, RAKR, QRTR, ATRH, ARRS, AKTR, NRQR, PRNT, e.g. RRTA or HRRK. All of these sequences have been shown to be compatible with Kv1.3 inhibitor activity and selectivity when added to the C-terminus of Odk2 analogues (see WO 12014/116937).

Thus the invention also provides an ion channel blocker having the formula:

R¹—Z¹—X—Z²—R²

where
R¹ is hydrogen, C₁₋₄ alkyl, acetyl, formyl, benzoyl or trifluoroacetyl;
R² is OH, NH₂ or CH₂OH,
X is a Kv1.3 inhibitor having the sequence of PaT1 or a variant thereof as described above, and
Z¹ and Z² are independently sequences of up to 10 amino acid residues;
with the proviso that the ion channel blocker have a maximum length of 50 amino acids;
or a pharmaceutically acceptable salt thereof.

As set out above, Z¹ may comprise or consist of the sequence GG or SG.

Additionally or alternatively, Z² may comprise or consist of the sequence RRTA, HRRK, QSKA, AGPR, RSRT, RHKR, GGKR, PKTA, TDAR, HRQQ, RPRH, ARNA, TGRK, HERT, NTRT, QRNG, AHRN, PRSA, QRQS, QRRK, ARAK, AKRD, RDKT, HRRK, RAKR, QRTR, ATRH, ARRS, AKTR, NRQR, PRNT, e.g. RRTA or HRRK.

Additionally or alternatively, R² can be CH₂OH and can be comprised in (4-amino-5-hydroxypentyl)guanidine or 4-amino-5-hydroxypentanamide.

The Kv1.3 inhibitor component of the molecule contains 6 cysteine (C) residues which together form three disulphide bonds, between residues 6C and 27C, residues 12C and 32C, and residues 16C and 34C. It is believed that these bonds are important for the conformation and activity of the Kv1.3 inhibitor.

The disulphide bonds may be indicated graphically as follows by reference to PaT1:

QMDMR[C(1)]SASVE[C(2)]KQK[C(3)]LKAIGSIFGK[C(1)]MN
KK[C(2)]K[C(3)]YPR

where a pair of cysteine residues which participate together in a disulphide bond are indicated by the same numeral in parentheses. Similar notation can be applied to any of the other sequences in this application. Except where the context demands otherwise, it should be understood that an active inhibitor compound includes appropriate disulphide bonding.

It may be desirable that no other cysteine residues are introduced into the Kv1.3 inhibitor component by substitution. Additionally or alternatively, it may be desirable that no cysteine residues are present in any N- or C-terminal additional sequence. Thus, in some embodiments, the molecule contains no other cysteine residues apart from those at positions 6, 12, 16, 27, 32 and 34 of PaT1.

However, in some embodiments, the inhibitor component comprises 8 cysteine residues, which together form four disulphide bonds, between residues 6C and 27C, residues 12C and 32C, residues 16C and 34C, and cysteine residues at positions corresponding to positions 22 and 37 of Pat1. Compound 87 is an example of such a molecule, and the disulphide bonding may be indicated graphically as follows:

H-S[Nie]D[Nle]R[C(1)]SASVE[C(2)]KQK[C(3)]LAAIG
[C(4)]IFGK[C(1)][Nle]NKK[C(2)]K[C(3)]YP[C(4)]-NH₂

Similar notation may be applied to other compounds having four pairs of disulphide bonds.

Thus, an inhibitor typically has cysteine residues at positions 22 and 37, or neither of positions 22 and 37 is a cysteine residue (i.e. both positions 22 and 37 are not cysteine).

The Kv1.3 inhibitor component may comprise one of the following sequences:

QMDMRCSASVECKQKCLKAIGSIFGKCMNKKCKCYPR
QMDMRCSASVECKQKCLKAIGRGFGKCMNKKCKCYPR
NMDMRCSASVECKQKCLKAIGSIFGKCMNKKCKCYPR
NMDMRCSASVECKQKCLKAIGRGFGKCMNKKCKCYPR
MDMRCSASVECKQKCLKAIGSIFGKCMNKKCKCYPR
DMRCSASVECKQKCLKAIGSIFGKCMNKKCKCYPR
NMDMRCSASVECKQKCLKAIGSIFGKCMNKKCKCYPR
NIDMRCSASVECKQKCLKAIGSIFGKCMNKKCKCYPR
NMDVRCSASVECKQKCLKAIGSIFGKCMNKKCKCYPR
NMDMRCSASVECKQKCKDAIGSIFGKCMNKKCKCYPR
NMEMRCSASVECKQKCLKAIGSIFGKCMNKKCKCYPR
N[Nle]DMRCSASVECKQKCLKAIGSIFGKCMNKKCKCYPR
NMD[Nle]RCSASVECKQKCLKAIGSIFGKCMNKKCKCYPR
NMDMRCSASVECKQKCLKAIGSIFGKC[Nle]NKKCKCYPR
NMDMRCSASVECKVKCLKAIGSIFGKCMNKKCKCYPR
NMDMRCSASVECKQLCLKAIGSIFGKCMNKKCKCYPR
NMDMRCSASVECKQKCKKAIGSIFGKCMNKKCKCYPR
NMDMRCSASVECKQKCLDAIGSIFGKCMNKKCKCYPR
NMDMRCSASVECKQKCLKAIRSIFGKCMNKKCKCYPR
NMDMRCSASVECKQKCLKAIESIFGKCMNKKCKCYPR
NMDMRCKASVECKQKCLKAIGSIFGKCMNKKCKCYPR
NMDMRCSISVECKQKCLKAIGSIFGKCMNKKCKCYPR
NMDMRCSASRECKQKCLKAIGSIFGKCMNKKCKCYPR
NMDMRCSASVQCKQKCLKAIGSIFGKCMNKKCKCYPR
NMDMRCSASVECLQKCLKAIGSIFGKCMNKKCKCYPR
NMDMRCSASVECAQKCLKAIGSIFGKCMNKKCKCYPR
NMDMRCSASVECKEKCLKAIGSIFGKCMNKKCKCYPR
NMDMRCSASVECKLKCLKAIGSIFGKCMNKKCKCYPR
NMDMRCSASVECKQKCLKAIHSIFGKCMNKKCKCYPR
NMDMRCSASVECKQKCLKAIGSKFGKCMNKKCKCYPR
NMDMRCSASVECKQKCLKAIGSRFGKCMNKKCKCYPR
NMDMRCSASVECKQKCLKAIGSIFGKCMNGKCKCYPR
NMDMRCSASVECKQKCLKAIGSIFGKCMNKKCHCYPR
NMDMRCSASVECKQKCLKAIGSIFGKCMNKKCVCYPR
N[Nle]D[Nle]RCSASVECKQKCLKAIGSIFGKCMNKKCKCYPR
N[Nle]D[Nle]RCSASVECKQKCLKAIGSIFGKC[Nle]NKKCKCYPR
P[Nle]D[Nle]RCSASVECKQKCLKAIGSIFGKC[Nle]NKKCKCYPR
N[Nie]D[Nle]RCRASVECKQKCLKAIGSIFGKC[Nle]NKKCKCYPR
N[Nle]D[Nle]RCSASVECEQKCLKAIGSIFGKC[Nle]NKKCKCYPR
N[Nle]D[Nle]RCSASVECQQKCLKAIGSIFGKC[Nle]NKKCKCYPR
N[Nle]D[Nle]RCSASVECKKKCLKAIGSIFGKC[Nle]NKKCKCYPR
N[Nle]S[Nle]RCSASVECKQKCLKAIGSIFGKC[Nle]NKKCKCYPR
N[Nle]D[Nle]RCSHSVECKQKCLKAIGSIFGKC[Nle]NKKCKCYPR
N[Nle]D[Nle]RCSASVECKQSCLKAIGSIFGKC[Nle]NKKCKCYPR
N[Nle]D[Nle]RCSASVECKQKCKAIGSIFGKC[Nle]NKKCKCYPR
NMDMRCSASVECKQKCYKAIGSIFGKCMNKKCKCYPR
NMDMRCSASVECKQKCRKAIGSIFGKCMNKKCKCYPR
NMDMRCSASVECKQKCLAAIGSIFGKCMNKKCKCYPR
NMDMRCSASVECKQKCLYAIGSIFGKCMNKKCKCYPR
NMDMRCSASVECKQKCLAIGSIFGKCMNKKCKCYPR
NMDMRCSASVECKQKCLKYIGSIFGKCMNKKCKCYPR
NMDMRCSASVECKQKCLKRIGSIFGKCMNKKCKCYPR
NMDMRCSASVECKQKCLKIGSIFGKCMNKKCKCYPR
NMDMRCSASVECKQKCLKAYGSIFGKCMNKKCKCYPR
NMDMRCSASVECKQKCLKAEGSIFGKCMNKKCKCYPR
NMDMRCSASVECKQKCLKARGSIFGKCMNKKCKCYPR
NMDMRCSASVECKQKCLKAGSIFGKCMNKKCKCYPR
N[Nle]D[Nle]RCSASVECKQKCLKAIGSPFGKC[Nle]NKKCKCYPR
N[Nle]D[Nle]RCSASKECKQKCLKAIGSIFGKC[Nle]NKKCKCYPR
H[Nle]D[Nle]RCSASVECKQKCLKAIGSIFGKC[Nle]NKKCKCYPR
Y[Nle]D[Nle]RCSASVECKQKCLKAIGSIFGKC[Nle]NKKCKCYPR
S[Nle]D[Nle]RCSASVECKQKCLKAIGSIFGKC[Nle]NKKCKCYPR
V[Nle]D[Nle]RCSASVECKQKCLKAIGSIFGKC[Nle]NKKCKCYPR
S[Nie]D[Nle]RCSA[Abu]VECKQKCLKAIGSIFGKCMNKKCKCYPR
S[Nle]D[Nle]RCSASVECKQKCLKAIGSIFG[homo-Lys]CMNKKCKCYPR
S[Nle]D[Nle]RCSASVECKQKCLKAIGSIFGKCMNKKCKC[F(4-NH₂)]PR
S[Nle]D[Nle]RCSASVECGQKCLKAIGSIFGKCMNKKCKCYPR
S[Nle]D[Nle]RCSASVECVQKCLKAIGSIFGKCMNKKCKCYPR
S[Nle]D[Nle]RCSASVECK[2-Amino-5-
carboxypentanoyl]KCLKAIGSIFGKCMNKKCKCYPR
S[Nle]D[Nle]RCSASVECKQKCLKAIGSIFGKCMNKKCKCYQ
S[Nle]D[Nle]RCSASVECKQKCLKAIGSIFGKCMNKKCRCYPR
S[Nle]DERCSASVECKQKCLKAIGSIFGKCMNKKCKCYPR
S[Nle]D[Nle]RCSASVECKQKCLGAIGSIFGKCMNKKCKCYPR
S[Nle]D[Nle]RCSASVECKQKCLVAIGSIFGKCMNKKCKCYPR
S[Nle]D[Nle]RCSASVECAQSCLKAIGSIFGKCMNKKCKCYPR
S[Nle]D[Nle]RCSASVECAQKCLAAIGSIFGKCMNKKCKCYPR
S[Nle]D[Nle]RCSASVECAQLCLAAIGSIFGKCMNKKCKCYPR
S[Nle]D[Nle]RCSASVECKQKCLKAIGSIFGKC[Nle]NKKCKCYPR
P[Nle]D[Nle]RCSASVECKQKCLKAIGCIFGKC[Nle]NKKCKCYPC
S[Nle]D[Nle]RCSASVECKEKCLQAIGSIFGKC[Nle]NKKCKCYPR
P[Nie]D[Nle]RCSASVECKEKCL[homo-Gln]AIGSIFGKC[Nle]NKKCKCYPR
CSASVECKQKCLKAIGCIFGKC[Nle]NKKCKCYPC
S[Nle]D[Nle]RCSASVECKQKCLAAIGCIFGKC[Nle]NKKCKCYPC
P[Nle]D[Nle]RCSASVECKQKCLKAIGCIFGKC[Nle]NKKCKCYPC
S[Nle]D[Nle]RCSALVECKQKCLKAIGSIFGKCMNKKCKCYPR
S[Nie]D[Nle]RCSAVVECKQKCLKAIGSIFGKCMNKKCKC(3)YPR
S[Nle]D[Nle]RCSASVECKQKCLKAIGSIFGKCMNKKCKC[F(4-F)]PR
S[Nle]D[Nle]RCSASVECKQKCLKAIGSIFGKCMNKKCKC[F(4-NO₂)]PR
S[Nle]D[Nle]RCSASVECKQKCLKAIGSIFGKCMNKKCKC[F(4-CH₃)]PR
[Nle]D[Nle]RCSASVECKQKCLKAIGSIFGKC[Nle]NKKCKCYPR
NID[Nle]RCSASVECKQKCLKAIGSIFGKC[Nle]NKKCKCYPR
PIE[Nle]RCSASVECKQKCLKAIGSIFGKC[Nle]NKKCKCYPR
P[Nle]E[Nle]RCSASVECKQKCLAAIGSIFGKC[Nle]NKKCKCYPR
P[Nle]E[Nle]RCSASVECKQKCLAAIGSIFGKC[Nle]NKKCKCYPR
P[Nle]E[Nle]RCSASVECKQKCLLAIGSIFGKC[Nle]NKKCKCYPR
PIDERCSASVECKQKCLAAIGSIFGKC[Nle]NKKCKCYPR
PIE[Nle]RCSASVECKQKCLAAIGSIFGKC[Nle]NKKCKCYPR
P[Nle]D[Nle]RCSASVECAQKCLAAIGSIFGKCMNKKCKCYPR
P[Nle]E[Nle]RCSASVECAQKCLAAIGSIFGKC[Nle]NKKCKCYPR
CSASVECKQKCLKAIGSIFGKC[Nle]NKKCKCYPR
CSASVECKQKCLKAIGSIFGKC[Nle]NKKCKCYPR
RCSASVECKQKCLKAIGSIFGKC[Nle]NKKCKCYPR
SKCSASVECKQKCLKAIGSIFGKC[Nle]NKKCKCYPR
LRCSASVECKQKCLKAIGSIFGKC[Nle]NKKCKCYPR
CSASVECKQKCLKAIGSIFGKC[Nle]NKKCKCYPS
CSASVECKQKCLKAIGSIFGKC[Nle]NKKCKCYS
CSASVECKQKCLKAIGSIFGKC[Nle]NKKCKCYG-NH2
CSASVECKQKCLKAIGSIFGKC[Nle]NKKCKCY-NH2
RCSASVECKQKCLKAIGSIFGKC[Nle]NKKCKCYPR
LRCSASVECKQKCLKAIGSIFGKC[Nle]NKKCKCYPR
LRCSASVECKQKCLAAIGSIFGKC[Nle]NKKCKCYPR
LRCSASVECKQKCLAAIGSIFGKCMNKKCKCYPR
CSASVECKQKCLKAIGSIFGKCMNKKCKCYPR
P[Nle]E[Nle]RCSASVECKEKCLAAIGSIFGKC[Nle]NKKCKCYPR
p[Nle]E[Nle]RCSASVECKQKCLAAIGSIFGKC[Nle]NKKCKCYPR
P[Nle]E[Nle]RCSASVECKQKCLAAIGSIFGKC[Nle]NKKCKCYR
[Nle]E[Nle]RCSASVECKQKCLAAIGSIFGKC[Nle]NKKCKCYPR
P[Nle]E[Nle]RCSASVECKQKCLAAIGSIFGKC[Nle]NKKCKCYPRR
P[Nie]E[Nle]RCSASVECKQKCLAAIGSIFGKC[Nle]NKKCKCYPRY
P[Nie]E[Nle]RCSASVECKQKCLAAIGSIFGKC[Nle]NKKCKCYPRL
P[Nle]E[Nle]RCSASVECKQKCLAAIGSIFGKC[Nle]NKKCKCYPRH
P[Nle]E[Nle]RCSASVECKQKCLAAIGSIFGKC[Nle]NKKCKCYPRE
P[Nle]E[Nle]RCSASVECKQKCLAAIGSIFGKC[Nle]NKKCKCYPRKS
P[Nle]E[Nle]RCSASVECKQKCLAAIGSIFGKC[Nle]NKKCKCYPRFE
P[Nle]E[Nle]RCSASVECKQKCLAAIGSIFGKC[Nle]NKKCKCYPRHR
P[Nle]E[Nle]RCSASVECKQKCLAAIGSIFGKC[Nle]NKKCKCYPRAK
P[Nle]E[Nle]RCSASVECKQKCLAAIGSIFGKC[Nle]NKKCKCYP-[(4-amino-
5-hydroxypentyl)guanidine]
P[Nle]E[Nle]RCSASVECKQKCLAAIGSIFGKC[Nle]NKKCKCYPR-[4-
amino-5-hydroxypentanamide]
P[Nle]E[Nle]RCSASVECKQKCLAAIGSIFGKC[Nle]NKKCKCYPRST
P[Nle]E[Nle]RCSASVECKQKCLAAIGSIFGKC[Nle]NKKCKCYPRRY
P[Nle]E[Nle]RCSSSVECKQKCLAAIGSIFGKC[Nle]NKKCKCYPR
P[Nle]E[Nle]RCSLSVECKQKCLAAIGSIFGKC[Nle]NKKCKCYPR
P[Nle]E[Nle]RCSAPVECKQKCLAAIGSIFGKC[Nle]NKKCKCYPR
P[Nle]E[Nle]RCSASPECKQKCLAAIGSIFGKC[Nle]NKKCKCYPR
P[Nle]E[Nle]RCSASQECKQKCLAAIGSIFGKC[Nle]NKKCKCYPR
P[Nle]E[Nle]RCSASVECLQKCLAAIGSIFGKC[Nle]NKKCKCYPR
P[Nle]E[Nle]RCSASVECKQPCLAAIGSIFGKC[Nle]NKKCKCYPR
P[Nle]E[Nle]RCEASVECKQKCLAAIGSIFGKC[Nle]NKKCKCYPR
P[Nle]E[Nle]RCFASVECKQKCLAAIGSIFGKC[Nle]NKKCKCYPR
P[Nle]E[Nle]RCSYSVECKQKCLAAIGSIFGKC[Nle]NKKCKCYPR
P[Nle]E[Nle]RCSAFVECKQKCLAAIGSIFGKC[Nle]NKKCKCYPR

Ion channel blockers may comprise or consist of any of the following sequences:

QMDMRCSASVECKQKCLKAIGSIFGKCMNKKCKCYPR
QMDMRCSASVECKQKCLKAIGRGFGKCMNKKCKCYPR
NMDMRCSASVECKQKCLKAIGSIFGKCMNKKCKCYPR
NMDMRCSASVECKQKCLKAIGRGFGKCMNKKCKCYPR
MDMRCSASVECKQKCLKAIGSIFGKCMNKKCKCYPR
DMRCSASVECKQKCLKAIGSIFGKCMNKKCKCYPR
NMDMRCSASVECKQKCLKAIGSIFGKCMNKKCKCYPR
NIDMRCSASVECKQKCLKAIGSIFGKCMNKKCKCYPR
NMDVRCSASVECKQKCLKAIGSIFGKCMNKKCKCYPR
NMDMRCSASVECKQKCKDAIGSIFGKCMNKKCKCYPR
GGNMDMRCSASVECKQKCLKAIGSIFGKCMNKKCKCYPR
NMEMRCSASVECKQKCLKAIGSIFGKCMNKKCKCYPR
SGNMDMRCSASVECKQKCLKAIGSIFGKCMNKKCKCYPR
N[Nle]DMRCSASVECKQKCLKAIGSIFGKCMNKKCKCYPR
NMD[Nle]RCSASVECKQKCLKAIGSIFGKCMNKKCKCYPR
NMDMRCSASVECKQKCLKAIGSIFGKC[Nle]NKKCKCYPR
NMDMRCSASVECKVKCLKAIGSIFGKCMNKKCKCYPR
NMDMRCSASVECKQLCLKAIGSIFGKCMNKKCKCYPR
NMDMRCSASVECKQKCKKAIGSIFGKCMNKKCKCYPR
NMDMRCSASVECKQKCLDAIGSIFGKCMNKKCKCYPR
NMDMRCSASVECKQKCLKAIRSIFGKCMNKKCKCYPR
NMDMRCSASVECKQKCLKAIESIFGKCMNKKCKCYPR
NMDMRCSASVECKQKCLKAIGSIFGKCMNKKCKCYPRRRTA
NMDMRCSASVECKQKCLKAIGSIFGKCMNKKCKCYPRHRRK
NMDMRCKASVECKQKCLKAIGSIFGKCMNKKCKCYPR
NMDMRCSISVECKQKCLKAIGSIFGKCMNKKCKCYPR
NMDMRCSASRECKQKCLKAIGSIFGKCMNKKCKCYPR
NMDMRCSASVQCKQKCLKAIGSIFGKCMNKKCKCYPR
NMDMRCSASVECLQKCLKAIGSIFGKCMNKKCKCYPR
NMDMRCSASVECAQKCLKAIGSIFGKCMNKKCKCYPR
NMDMRCSASVECKEKCLKAIGSIFGKCMNKKCKCYPR
NMDMRCSASVECKLKCLKAIGSIFGKCMNKKCKCYPR
NMDMRCSASVECKQKCLKAIHSIFGKCMNKKCKCYPR
NMDMRCSASVECKQKCLKAIGSKFGKCMNKKCKCYPR
NMDMRCSASVECKQKCLKAIGSRFGKCMNKKCKCYPR
NMDMRCSASVECKQKCLKAIGSIFGKCMNGKCKCYPR
NMDMRCSASVECKQKCLKAIGSIFGKCMNKKCHCYPR
NMDMRCSASVECKQKCLKAIGSIFGKCMNKKCVCYPR
N[Nle]D[Nle]RCSASVECKQKCLKAIGSIFGKCMNKKCKCYPR
N[Nle]D[Nle]RCSASVECKQKCLKAIGSIFGKC[Nle]NKKCKCYPR
P[Nle]D[Nle]RCSASVECKQKCLKAIGSIFGKC[Nle]NKKCKCYPR
N[Nle]D[Nle]RCRASVECKQKCLKAIGSIFGKC[Nle]NKKCKCYPR
N[Nle]D[Nle]RCSASVECEQKCLKAIGSIFGKC[Nle]NKKCKCYPR
N[Nle]D[Nle]RCSASVECQQKCLKAIGSIFGKC[Nle]NKKCKCYPR
N[Nle]D[Nle]RCSASVECKKKCLKAIGSIFGKC[Nle]NKKCKCYPR
N[Nle]S[Nle]RCSASVECKQKCLKAIGSIFGKC[Nle]NKKCKCYPR
N[Nie]D[Nle]RCSHSVECKQKCLKAIGSIFGKC[Nle]NKKCKCYPR
N[Nle]D[Nle]RCSASVECKQSCLKAIGSIFGKC[Nle]NKKCKCYPR
N[Nle]D[Nle]RCSASVECKQKCKAIGSIFGKC[Nle]NKKCKCYPR
NMDMRCSASVECKQKCYKAIGSIFGKCMNKKCKCYPR
NMDMRCSASVECKQKCRKAIGSIFGKCMNKKCKCYPR
NMDMRCSASVECKQKCLAAIGSIFGKCMNKKCKCYPR
NMDMRCSASVECKQKCLYAIGSIFGKCMNKKCKCYPR
NMDMRCSASVECKQKCLAIGSIFGKCMNKKCKCYPR
NMDMRCSASVECKQKCLKYIGSIFGKCMNKKCKCYPR
NMDMRCSASVECKQKCLKRIGSIFGKCMNKKCKCYPR
NMDMRCSASVECKQKCLKIGSIFGKCMNKKCKCYPR
NMDMRCSASVECKQKCLKAYGSIFGKCMNKKCKCYPR
NMDMRCSASVECKQKCLKAEGSIFGKCMNKKCKCYPR
NMDMRCSASVECKQKCLKARGSIFGKCMNKKCKCYPR
NMDMRCSASVECKQKCLKAGSIFGKCMNKKCKCYPR
N[Nle]D[Nle]RCSASVECKQKCLKAIGSPFGKC[Nle]NKKCKCYPR
N[Nle]D[Nle]RCSASKECKQKCLKAIGSIFGKC[Nle]NKKCKCYPR
H[Nle]D[Nle]RCSASVECKQKCLKAIGSIFGKC[Nle]NKKCKCYPR
Y[Nle]D[Nle]RCSASVECKQKCLKAIGSIFGKC[Nle]NKKCKCYPR
S[Nle]D[Nle]RCSASVECKQKCLKAIGSIFGKC[Nle]NKKCKCYPR
V[Nie]D[Nle]RCSASVECKQKCLKAIGSIFGKC[Nle]NKKCKCYPR
S[Nie]D[Nle]RCSA[Abu]VECKQKCLKAIGSIFGKCMNKKCKCYPR
S[Nle]D[Nle]RCSASVECKQKCLKAIGSIFG[homo-Lys]CMNKKCKCYPR
S[Nle]D[Nle]RCSASVECKQKCLKAIGSIFGKCMNKKCKC[F(4-NH2)]PR
S[Nle]D[Nle]RCSASVECGQKCLKAIGSIFGKCMNKKCKCYPR
S[Nle]D[Nle]RCSASVECVQKCLKAIGSIFGKCMNKKCKCYPR
S[Nle]D[Nle]RCSASVECK[2-Amino-5-
carboxypentanoyl]KCLKAIGSIFGKCMNKKCKCYPR
S[Nle]D[Nle]RCSASVECKQKCLKAIGSIFGKCMNKKCKCYQ
S[Nle]D[Nle]RCSASVECKQKCLKAIGSIFGKCMNKKCRCYPR
S[Nle]DERCSASVECKQKCLKAIGSIFGKCMNKKCKCYPR
S[Nie]D[Nle]RCSASVECKQKCLGAIGSIFGKCMNKKCKCYPR
S[Nle]D[Nle]RCSASVECKQKCLVAIGSIFGKCMNKKCKCYPR
S[Nle]D[Nle]RCSASVECAQSCLKAIGSIFGKCMNKKCKCYPR
S[Nle]D[Nle]RCSASVECAQKCLAAIGSIFGKCMNKKCKCYPR
S[Nie]D[Nle]RCSASVECAQLCLAAIGSIFGKCMNKKCKCYPR
S[Nle]D[Nle]RCSASVECKQKCLKAIGSIFGKC[Nle]NKKCKCYPR
P[Nle]D[Nle]RCSASVECKQKCLKAIGCIFGKC[Nle]NKKCKCYPC
S[Nle]D[Nle]RCSASVECKEKCLQAIGSIFGKC[Nle]NKKCKCYPR
P[Nle]D[Nle]RCSASVECKEKCL[homo-Gln]AIGSIFGKC[Nle]NKKCKCYPR
CSASVECKQKCLKAIGCIFGKC[Nle]NKKCKCYPC
S[Nle]D[Nle]RCSASVECKQKCLAAIGCIFGKC[Nle]NKKCKCYPC
P[Nle]D[Nle]RCSASVECKQKCLKAIGCIFGKC[Nle]NKKCKCYPC
S[Nle]D[Nle]RCSALVECKQKCLKAIGSIFGKCMNKKCKCYPR
S[Nle]D[Nle]RCSAVVECKQKCLKAIGSIFGKCMNKKCKCYPR
S[Nle]D[Nle]RCSASVECKQKCLKAIGSIFGKCMNKKCKC[F(4-F)]PR
S[Nle]D[Nle]RCSASVECKQKCLKAIGSIFGKCMNKKCKC[F(4-NO₂)]PR
S[Nle]D[Nle]RCSASVECKQKCLKAIGSIFGKCMNKKCKC[F(4-CH₃)]PR
[Nle]D[Nle]RCSASVECKQKCLKAIGSIFGKC[Nle]NKKCKCYPR
NID[Nle]RCSASVECKQKCLKAIGSIFGKC[Nle]NKKCKCYPR
PIE[Nle]RCSASVECKQKCLKAIGSIFGKC[Nle]NKKCKCYPR
P[Nie]E[Nle]RCSASVECKQKCLAAIGSIFGKC[Nle]NKKCKCYPR
P[Nle]E[Nle]RCSASVECKQKCLAAIGSIFGKC[Nle]NKKCKCYPR
P[Nle]E[Nle]RCSASVECKQKCLLAIGSIFGKC[Nle]NKKCKCYPR
PIDERCSASVECKQKCLAAIGSIFGKC[Nle]NKKCKCYPR
PIE[Nle]RCSASVECKQKCLAAIGSIFGKC[Nle]NKKCKCYPR
P[Nle]D[Nle]RCSASVECAQKCLAAIGSIFGKCMNKKCKCYPR
P[Nle]E[Nle]RCSASVECAQKCLAAIGSIFGKC[Nle]NKKCKCYPR
CSASVECKQKCLKAIGSIFGKC[Nle]NKKCKCYPR
CSASVECKQKCLKAIGSIFGKC[Nle]NKKCKCYPR
RCSASVECKQKCLKAIGSIFGKC[Nle]NKKCKCYPR
SKCSASVECKQKCLKAIGSIFGKC[Nle]NKKCKCYPR
LRCSASVECKQKCLKAIGSIFGKC[Nle]NKKCKCYPR
CSASVECKQKCLKAIGSIFGKC[Nle]NKKCKCYPS
CSASVECKQKCLKAIGSIFGKC[Nle]NKKCKCYS
CSASVECKQKCLKAIGSIFGKC[Nle]NKKCKCYG-NH2
CSASVECKQKCLKAIGSIFGKC[Nle]NKKCKCY-NH2
RCSASVECKQKCLKAIGSIFGKC[Nle]NKKCKCYPR
LRCSASVECKQKCLKAIGSIFGKC[Nle]NKKCKCYPR
LRCSASVECKQKCLAAIGSIFGKC[Nle]NKKCKCYPR
LRCSASVECKQKCLAAIGSIFGKCMNKKCKCYPR
CSASVECKQKCLKAIGSIFGKCMNKKCKCYPR
P[Nle]E[Nle]RCSASVECKEKCLAAIGSIFGKC[Nle]NKKCKCYPR
p[Nle]E[Nle]RCSASVECKQKCLAAIGSIFGKC[Nle]NKKCKCYPR
P[Nle]E[Nle]RCSASVECKQKCLAAIGSIFGKC[Nle]NKKCKCYR
[Nle]E[Nle]RCSASVECKQKCLAAIGSIFGKC[Nle]NKKCKCYPR
P[Nle]E[Nle]RCSASVECKQKCLAAIGSIFGKC[Nle]NKKCKCYPRR
P[Nle]E[Nle]RCSASVECKQKCLAAIGSIFGKC[Nle]NKKCKCYPRY
P[Nle]E[Nle]RCSASVECKQKCLAAIGSIFGKC[Nle]NKKCKCYPRL
P[Nie]E[Nle]RCSASVECKQKCLAAIGSIFGKC[Nle]NKKCKCYPRH
P[Nle]E[Nle]RCSASVECKQKCLAAIGSIFGKC[Nle]NKKCKCYPRE
P[Nle]E[Nle]RCSASVECKQKCLAAIGSIFGKC[Nle]NKKCKCYPRKS
P[Nle]E[Nle]RCSASVECKQKCLAAIGSIFGKC[Nle]NKKCKCYPRFE
P[Nle]E[Nle]RCSASVECKQKCLAAIGSIFGKC[Nle]NKKCKCYPRHR
P[Nle]E[Nle]RCSASVECKQKCLAAIGSIFGKC[Nle]NKKCKCYPRAK
P[Nle]E[Nle]RCSASVECKQKCLAAIGSIFGKC[Nle]NKKCKCYP-[(4-amino-
5-hydroxypentyl)guanidine]
P[Nle]E[Nle]RCSASVECKQKCLAAIGSIFGKC[Nle]NKKCKCYPR-[4-
amino-5-hydroxypentanamide]
P[Nle]E[Nle]RCSASVECKQKCLAAIGSIFGKC[Nle]NKKCKCYPRST
P[Nle]E[Nle]RCSASVECKQKCLAAIGSIFGKC[Nle]NKKCKCYPRRY
P[Nle]E[Nle]RCSSSVECKQKCLAAIGSIFGKC[Nle]NKKCKCYPR
P[Nle]E[Nle]RCSLSVECKQKCLAAIGSIFGKC[Nle]NKKCKCYPR
P[Nle]E[Nle]RCSAPVECKQKCLAAIGSIFGKC[Nle]NKKCKCYPR
P[Nle]E[Nle]RCSASPECKQKCLAAIGSIFGKC[Nle]NKKCKCYPR
P[Nle]E[Nle]RCSASQECKQKCLAAIGSIFGKC[Nle]NKKCKCYPR
P[Nle]E[Nle]RCSASVECLQKCLAAIGSIFGKC[Nle]NKKCKCYPR
P[Nle]E[Nle]RCSASVECKQPCLAAIGSIFGKC[Nle]NKKCKCYPR
P[Nle]E[Nle]RCEASVECKQKCLAAIGSIFGKC[Nle]NKKCKCYPR
P[Nle]E[Nle]RCFASVECKQKCLAAIGSIFGKC[Nle]NKKCKCYPR
P[Nle]E[Nle]RCSYSVECKQKCLAAIGSIFGKC[Nle]NKKCKCYPR
P[Nle]E[Nle]RCSAFVECKQKCLAAIGSIFGKC[Nle]NKKCKCYPR

In some embodiments, the ion channel blocker does not have the sequence:

QMDMRCSASVECKQKCLKAIGSIFGKCMNKKCKCYPR
or
QMDMRCSASVECKQKCLKAIGRGFGKCMNKKCKCYPR.

In some embodiments, R¹ is H and R² is NH₂ or OH. In some embodiments, it may be desirable that R² is OH.

Ion channel blockers include the following:

(Cpd. 1)
H-QMDMRCSASVECKQKCLKAIGSIFGKCMNKKCKCYPR-OH
(Cpd. 2)
H-QMDMRCSASVECKQKCLKAIGRGFGKCMNKKCKCYPR-OH
(Cpd. 3)
H-NMDMRCSASVECKQKCLKAIGSIFGKCMNKKCKCYPR-OH
(Cpd. 4)
H-NMDMRCSASVECKQKCLKAIGRGFGKCMNKKCKCYPR-OH
(Cpd. 5)
H-MDMRCSASVECKQKCLKAIGSIFGKCMNKKCKCYPR-OH
(Cpd. 6)
H-DMRCSASVECKQKCLKAIGSIFGKCMNKKCKCYPR-OH
(Cpd. 7)
H-NMDMRCSASVECKQKCLKAIGSIFGKCMNKKCKCYPR-NH₂
(Cpd. 8)
H-NIDMRCSASVECKQKCLKAIGSIFGKCMNKKCKCYPR-OH
(Cpd. 9)
H-NMDVRCSASVECKQKCLKAIGSIFGKCMNKKCKCYPR-OH
(Cpd. 10)
H-NMDMRCSASVECKQKCKDAIGSIFGKCMNKKCKCYPR-OH
(Cpd. 11)
H-GGNMDMRCSASVECKQKCLKAIGSIFGKCMNKKCKCYPR-OH
(Cpd. 12)
H-NMEMRCSASVECKQKCLKAIGSIFGKCMNKKCKCYPR-OH
(Cpd. 13)
H-SGNMDMRCSASVECKQKCLKAIGSIFGKCMNKKCKCYPR-OH
(Cpd. 14)
H-N[Nle]DMRCSASVECKQKCLKAIGSIFGKCMNKKCKCYPR-NH₂
(Cpd. 15)
H-NMD[Nle]RCSASVECKQKCLKAIGSIFGKCMNKKCKCYPR-NH₂
(Cpd. 16)
H-NMDMRCSASVECKQKCLKAIGSIFGKC[Nle]NKKCKCYPR-NH₂
(Cpd. 17)
H-NMDMRCSASVECKVKCLKAIGSIFGKCMNKKCKCYPR-NH₂
(Cpd. 18)
H-NMDMRCSASVECKQLCLKAIGSIFGKCMNKKCKCYPR-NH₂
(Cpd. 19)
H-NMDMRCSASVECKQKCKKAIGSIFGKCMNKKCKCYPR-NH₂
(Cpd. 20)
H-NMDMRCSASVECKQKCLDAIGSIFGKCMNKKCKCYPR-NH₂
(Cpd. 21)
H-NMDMRCSASVECKQKCLKAIRSIFGKCMNKKCKCYPR-NH₂
(Cpd. 22)
H-NMDMRCSASVECKQKCLKAIESIFGKCMNKKCKCYPR-NH₂
(Cpd. 23)
H-NMDMRCSASVECKQKCLKAIGSIFGKCMNKKCKCYPRRRTA-NH₂
(Cpd. 24)
H-NMDMRCSASVECKQKCLKAIGSIFGKCMNKKCKCYPRHRRK-NH₂
(Cpd. 25)
H-NMDMRCKASVECKQKCLKAIGSIFGKCMNKKCKCYPR-NH₂
(Cpd. 26)
H-NMDMRCSISVECKQKCLKAIGSIFGKCMNKKCKCYPR-NH₂
(Cpd. 27)
H-NMDMRCSASRECKQKCLKAIGSIFGKCMNKKCKCYPR-NH₂
(Cpd. 28)
H-NMDMRCSASVQCKQKCLKAIGSIFGKCMNKKCKCYPR-NH₂
(Cpd. 29)
H-NMDMRCSASVECLQKCLKAIGSIFGKCMNKKCKCYPR-NH₂
(Cpd. 30)
H-NMDMRCSASVECAQKCLKAIGSIFGKCMNKKCKCYPR-NH₂
(Cpd. 31)
H-NMDMRCSASVECKEKCLKAIGSIFGKCMNKKCKCYPR-NH₂
(Cpd. 32)
H-NMDMRCSASVECKLKCLKAIGSIFGKCMNKKCKCYPR-NH₂
(Cpd. 33)
H-NMDMRCSASVECKQKCLKAIHSIFGKCMNKKCKCYPR-NH₂
(Cpd. 34)
H-NMDMRCSASVECKQKCLKAIGSKFGKCMNKKCKCYPR-NH₂
(Cpd. 35)
H-NMDMRCSASVECKQKCLKAIGSRFGKCMNKKCKCYPR-NH₂
(Cpd. 36)
H-NMDMRCSASVECKQKCLKAIGSIFGKCMNGKCKCYPR-NH₂
(Cpd. 37)
H-NMDMRCSASVECKQKCLKAIGSIFGKCMNKKCHCYPR-NH₂
(Cpd. 38)
H-NMDMRCSASVECKQKCLKAIGSIFGKCMNKKCVCYPR-NH₂
(Cpd. 39)
H-N[Nle]D[Nle]RCSASVECKQKCLKAIGSIFGKCMNKKCKCYPR-NH₂
(Cpd. 40)
H-N[Nle]D[Nle]RCSASVECKQKCLKAIGSIFGKC[Nle]NKKCKCYPR-NH₂
(Cpd. 41)
H-P[Nle]D[Nle]RCSASVECKQKCLKAIGSIFGKC[Nle]NKKCKCYPR-NH₂
(Cpd. 42)
H-N[Nle]D[Nle]RCRASVECKQKCLKAIGSIFGKC[Nle]NKKCKCYPR-NH₂
(Cpd. 43)
H-N[Nle]D[Nle]RCSASVECEQKCLKAIGSIFGKC[Nle]NKKCKCYPR-NH₂
(Cpd. 44)
H-N[Nle]D[Nle]RCSASVECQQKCLKAIGSIFGKC[Nle]NKKCKCYPR-NH₂
(Cpd. 45)
H-N[Nle]D[Nle]RCSASVECKKKCLKAIGSIFGKC[Nle]NKKCKCYPR-NH₂
(Cpd. 46)
H-N[Nle]S[Nle]RCSASVECKQKCLKAIGSIFGKC[Nle]NKKCKCYPR-NH₂
(Cpd. 47)
H-N[Nle]D[Nle]RCSHSVECKQKCLKAIGSIFGKC[Nle]NKKCKCYPR-NH₂
(Cpd. 48)
H-N[Nle]D[Nle]RCSASVECKQSCLKAIGSIFGKC[Nle]NKKCKCYPR-NH₂
(Cpd. 49)
H-N[Nle]D[Nle]RCSASVECKQKCKAIGSIFGKC[Nle]NKKCKCYPR-NH₂
(Cpd. 50)
H-NMDMRCSASVECKQKCYKAIGSIFGKCMNKKCKCYPR-NH₂
(Cpd. 51)
H-NMDMRCSASVECKQKCRKAIGSIFGKCMNKKCKCYPR-NH₂
(Cpd. 52)
H-NMDMRCSASVECKQKCLAAIGSIFGKCMNKKCKCYPR-NH₂
(Cpd. 53)
H-NMDMRCSASVECKQKCLYAIGSIFGKCMNKKCKCYPR-NH₂
(Cpd. 54)
H-NMDMRCSASVECKQKCLAIGSIFGKCMNKKCKCYPR-NH₂
(Cpd. 55)
H-NMDMRCSASVECKQKCLKYIGSIFGKCMNKKCKCYPR-NH₂
(Cpd. 56)
H-NMDMRCSASVECKQKCLKRIGSIFGKCMNKKCKCYPR-NH₂
(Cpd. 57)
H-NMDMRCSASVECKQKCLKIGSIFGKCMNKKCKCYPR-NH₂
(Cpd. 58)
H-NMDMRCSASVECKQKCLKAYGSIFGKCMNKKCKCYPR-NH₂
(Cpd. 59)
H-NMDMRCSASVECKQKCLKAEGSIFGKCMNKKCKCYPR-NH₂
(Cpd. 60)
H-NMDMRCSASVECKQKCLKARGSIFGKCMNKKCKCYPR-NH₂
(Cpd. 61)
H-NMDMRCSASVECKQKCLKAGSIFGKCMNKKCKCYPR-NH₂
(Cpd. 62)
H-N[Nle]D[Nle]RCSASVECKQKCLKAIGSPFGKC[Nle]NKKCKCYPR-NH₂
(Cpd. 63)
H-N[Nle]D[Nle]RCSASKECKQKCLKAIGSIFGKC[Nle]NKKCKCYPR-NH₂
(Cpd. 64)
H-H[Nle]D[Nle]RCSASVECKQKCLKAIGSIFGKC[Nle]NKKCKCYPR-NH₂
(Cpd. 65)
H-Y[Nle]D[Nle]RCSASVECKQKCLKAIGSIFGKC[Nle]NKKCKCYPR-NH₂
(Cpd. 66)
H-S[Nle]D[Nle]RCSASVECKQKCLKAIGSIFGKC[Nle]NKKCKCYPR-NH₂
(Cpd. 67)
H-V[Nle]D[Nle]RCSASVECKQKCLKAIGSIFGKC[Nle]NKKCKCYPR-NH₂
(Cpd. 68)
H-S[Nle]D[Nle]RCSA[Abu]VECKQKCLKAIGSIFGKCMNKKCKCYPR-NH₂
(Cpd. 69)
H-S[Nle]D[Nle]RCSASVECKQKCLKAIGSIFG[homo-Lys]CMNKKCKCYPR-
NH₂
(Cpd. 70)
H-S[Nle]D[Nle]RCSASVECKQKCLKAIGSIFGKCMNKKCKC[F(4-NH₂)]PR-
NH₂
(Cpd. 71)
H-S[Nle]D[Nle]RCSASVECGQKCLKAIGSIFGKCMNKKCKCYPR-NH₂
(Cpd. 72)
H-S[Nle]D[Nle]RCSASVECVQKCLKAIGSIFGKCMNKKCKCYPR-NH₂
(Cpd. 73)
H-S[Nle]D[Nle]RCSASVECK[2-Amino-5-carboxypentanoyl]
KCLKAIGSIFGKCMNKKCKCYPR-NH₂
(Cpd. 74)
H-S[Nle]D[Nle]RCSASVECKQKCLKAIGSIFGKCMNKKCKCYQ-NH₂
(Cpd. 75)
H-S[Nle]D[Nle]RCSASVECKQKCLKAIGSIFGKCMNKKCRCYPR-NH₂
(Cpd. 76)
H-S[Nle]DERCSASVECKQKCLKAIGSIFGKCMNKKCKCYPR-NH₂
(Cpd. 77)
H-S[Nle]D[Nle]RCSASVECKQKCLGAIGSIFGKCMNKKCKCYPR-NH₂
(Cpd. 78)
H-S[Nle]D[Nle]RCSASVECKQKCLVAIGSIFGKCMNKKCKCYPR-NH₂
(Cpd. 79)
H-S[Nle]D[Nle]RCSASVECAQSCLKAIGSIFGKCMNKKCKCYPR-NH₂
(Cpd. 80)
H-S[Nle]D[Nle]RCSASVECAQKCLAAIGSIFGKCMNKKCKCYPR-NH₂
(Cpd. 81)
H-S[Nle]D[Nle]RCSASVECAQLCLAAIGSIFGKCMNKKCKCYPR-NH₂
(Cpd. 82)
H-S[Nle]D[Nle]RCSASVECKQKCLKAIGSIFGKC[Nle]NKKCKCYPR-OH
(Cpd. 83)
H-P[Nle]D[Nle]RCSASVECKQKCLKAIGCIFGKC[Nle]NKKCKCYPC-NH₂
(Cpd. 84)
H-S[Nle]D[Nle]RCSASVECKEKCLQAIGSIFGKC[Nle]NKKCKCYPR-NH₂
(Cpd. 85)
H-P[Nle]D[Nle]RCSASVECKEKCL[homo-Gln]AIGSIFGKC[Nle]
NKKCKCYPR-NH₂
(Cpd. 86)
H-CSASVECKQKCLKAIGCIFGKC[Nle]NKKCKCYPC-NH₂
(Cpd. 87)
H-S[Nle]D[Nle]RCSASVECKQKCLAAIGCIFGKC[Nle]NKKCKCYPC-
NH₂
(Cpd. 88)
H-P[Nle]D[Nle]RCSASVECKQKCLKAIGCIFGKC[Nle]NKKCKCYPC-
OH
(Cpd. 89)
H-S[Nle]D[Nle]RCSALVECKQKCLKAIGSIFGKCMNKKCKCYPR-NH₂
(Cpd. 90)
H-S[Nle]D[Nle]RCSAVVECKQKCLKAIGSIFGKCMNKKCKCYPR-NH₂
(Cpd. 91)
H-S[Nle]D[Nle]RCSASVECKQKCLKAIGSIFGKCMNKKCKC[F(4-F)]PR-
NH₂
(Cpd. 92)
H-S[Nle]D[Nle]RCSASVECKQKCLKAIGSIFGKCMNKKCKC[F(4-NO₂)]PR-
NH₂
(Cpd. 93)
H-S[Nle]D[Nle]RCSASVECKQKCLKAIGSIFGKCMNKKCKC[F(4-CH₃)]PR-
NH₂
(Cpd. 94)
H-[Nle]D[Nle]RCSASVECKQKCLKAIGSIFGKC[Nle]NKKCKCYPR-
OH
(Cpd. 95)
H-NID[Nle]RCSASVECKQKCLKAIGSIFGKC[Nle]NKKCKCYPR-OH
(Cpd. 96)
H-PIE[Nle]RCSASVECKQKCLKAIGSIFGKC[Nle]NKKCKCYPR-OH
(Cpd. 97)
H-P[Nle]E[Nle]RCSASVECKQKCLAAIGSIFGKC[Nle]NKKCKCYPR-
OH
(Cpd. 98)
H-P[Nle]E[Nle]RCSASVECKQKCLAAIGSIFGKC[Nle]NKKCKCYPR-
NH₂
(Cpd. 99)
H-P[Nle]E[Nle]RCSASVECKQKCLLAIGSIFGKC[Nle]NKKCKCYPR-
OH
(Cpd. 100)
H-PIDERCSASVECKQKCLAAIGSIFGKC[Nle]NKKCKCYPR-OH
(Cpd. 101)
H-PIE[Nle]RCSASVECKQKCLAAIGSIFGKC[Nle]NKKCKCYPR-OH
(Cpd. 102)
H-P[Nle]D[Nle]RCSASVECAQKCLAAIGSIFGKCMNKKCKCYPR-OH
(Cpd. 103)
H-P[Nle]E[Nle]RCSASVECAQKCLAAIGSIFGKC[Nle]NKKCKCYPR-OH
(Cpd. 104)
H-CSASVECKQKCLKAIGSIFGKC[Nle]NKKCKCYPR-NH₂
(Cpd. 105)
Ac-CSASVECKQKCLKAIGSIFGKC[Nle]NKKCKCYPR-NH₂
(Cpd. 106)
H-RCSASVECKQKCLKAIGSIFGKC[Nle]NKKCKCYPR-NH₂
(Cpd. 107)
Ac-SKCSASVECKQKCLKAIGSIFGKC[Nle]NKKCKCYPR-NH₂
(Cpd. 108)
Ac-LRCSASVECKQKCLKAIGSIFGKC[Nle]NKKCKCYPR-NH₂
(Cpd. 109)
Ac-CSASVECKQKCLKAIGSIFGKC[Nle]NKKCKCYPS-NH₂
(Cpd. 110)
Ac-CSASVECKQKCLKAIGSIFGKC[Nle]NKKCKCYS-NH₂
(Cpd. 111)
Ac-CSASVECKQKCLKAIGSIFGKC[Nle]NKKCKCYG-NH₂
(Cpd. 112)
Ac-CSASVECKQKCLKAIGSIFGKC[Nle]NKKCKCY-NH₂
(Cpd. 113)
Ac-RCSASVECKQKCLKAIGSIFGKC[Nle]NKKCKCYPR-OH
(Cpd. 114)
H-LRCSASVECKQKCLKAIGSIFGKC[Nle]NKKCKCYPR-OH
(Cpd. 115)
Ac-LRCSASVECKQKCLAAIGSIFGKC[Nle]NKKCKCYPR-OH
(Cpd. 116)
Ac-LRCSASVECKQKCLAAIGSIFGKCMNKKCKCYPR-OH
(Cpd. 117)
H-CSASVECKQKCLKAIGSIFGKCMNKKCKCYPR-OH
(Cpd. 118)
H-P[Nle]E[Nle]RCSASVECKEKCLAAIGSIFGKC[Nle]NKKCKCYPR-OH
(Cpd. 119)
H-p[Nle]E[Nle]RCSASVECKQKCLAAIGSIFGKC[Nle]NKKCKCYPR-OH
(Cpd. 120)
H-P[Nle]E[Nle]RCSASVECKQKCLAAIGSIFGKC[Nle]NKKCKCYR-OH
(Cpd. 121)
H-[Nle]E[Nle]RCSASVECKQKCLAAIGSIFGKC[Nle]NKKCKCYPR-OH
(Cpd. 122)
H-P[Nle]E[Nle]RCSASVECKQKCLAAIGSIFGKC[Nle]NKKCKCYPRR-
OH
(Cpd. 123)
H-P[Nle]E[Nle]RCSASVECKQKCLAAIGSIFGKC[Nle]NKKCKCYPRY-
OH
(Cpd. 124)
H-P[Nle]E[Nle]RCSASVECKQKCLAAIGSIFGKC[Nle]NKKCKCYPRL-
OH
(Cpd. 125)
H-P[Nle]E[Nle]RCSASVECKQKCLAAIGSIFGKC[Nle]NKKCKCYPRH-
OH
(Cpd. 126)
H-P[Nle]E[Nle]RCSASVECKQKCLAAIGSIFGKC[Nle]NKKCKCYPRE-
OH
(Cpd. 127)
H-P[Nle]E[Nle]RCSASVECKQKCLAAIGSIFGKC[Nle]NKKCKCYPRKS-
OH
(Cpd. 128)
H-P[Nle]E[Nle]RCSASVECKQKCLAAIGSIFGKC[Nle]NKKCKCYPRFE-
OH
(Cpd. 129)
H-P[Nle]E[Nle]RCSASVECKQKCLAAIGSIFGKC[Nle]NKKCKCYPRHR-
OH
(Cpd. 130)
H-P[Nle]E[Nle]RCSASVECKQKCLAAIGSIFGKC[Nle]NKKCKCYPRAK-
OH
(Cpd. 131)
H-P[Nle]E[Nle]RCSASVECKQKCLAAIGSIFGKC[Nle]NKKCKCYP-
[(4-amino-5-hydroxypentyl)guanidine]
(Cpd. 132)
H-P[Nle]E[Nle]RCSASVECKQKCLAAIGSIFGKC[Nle]NKKCKCYPR-
[4-amino-5-hydroxypentanamide]
(Cpd. 133)
H-P[Nle]E[Nle]RCSASVECKQKCLAAIGSIFGKC[Nle]NKKCKCYPRST-
OH
(Cpd. 134)
H-P[Nle]E[Nle]RCSASVECKQKCLAAIGSIFGKC[Nle]NKKCKCYPRRY-
OH
(Cpd. 135)
H-P[Nle]E[Nle]RCSSSVECKQKCLAAIGSIFGKC[Nle]NKKCKCYPR-
OH
(Cpd. 136)
H-P[Nle]E[Nle]RCSLSVECKQKCLAAIGSIFGKC[Nle]NKKCKCYPR-
OH
(Cpd. 137)
H-P[Nle]E[Nle]RCSAPVECKQKCLAAIGSIFGKC[Nle]NKKCKCYPR-
OH
(Cpd. 138)
H-P[Nle]E[Nle]RCSASPECKQKCLAAIGSIFGKC[Nle]NKKCKCYPR-
OH
(Cpd. 139)
H-P[Nle]E[Nle]RCSASQECKQKCLAAIGSIFGKC[Nle]NKKCKCYPR-
OH
(Cpd. 140)
H-P[Nle]E[Nle]RCSASVECLQKCLAAIGSIFGKC[Nle]NKKCKCYPR-
OH
(Cpd. 141)
H-P[Nle]E[Nle]RCSASVECKQPCLAAIGSIFGKC[Nle]NKKCKCYPR-
OH
(Cpd. 142)
H-P[Nle]E[Nle]RCEASVECKQKCLAAIGSIFGKC[Nle]NKKCKCYPR-
OH
(Cpd. 143)
H-P[Nle]E[Nle]RCFASVECKQKCLAAIGSIFGKC[Nle]NKKCKCYPR-
OH
(Cpd. 144)
H-P[Nle]E[Nle]RCSYSVECKQKCLAAIGSIFGKC[Nle]NKKCKCYPR-
OH
(Cpd. 145)
H-P[Nie]E[Nle]RCSAFVECKQKCLAAIGSIFGKC[Nle]NKKCKCYPR-
OH

In an embodiment, the ion channel blocker of the invention is:

(Cpd. 97)
H-P[Nle]E[Nle]RCSASVECKQKCLAAIGSIFGKC[Nle]
NKKCKCYPR-OH.

In some embodiments, the ion channel blocker is not:

(Cpd. 1)
H-QMDMRCSASVECKQKCLKAIGSIFGKCMNKKCKCYPR-OH
or
(Cpd. 2)
H-QMDMRCSASVECKQKCLKAIGRGFGKCMNKKCKCYPR-OH.

The invention further provides a nucleic acid encoding an ion channel blocker, a Kv1.3 inhibitor, or a peptide Z¹—X—Z² as described.

The invention also provides an expression vector comprising a nucleic acid of the invention.

The invention further comprises a host cell comprising a nucleic acid or an expression vector of the invention, and capable of expressing an ion channel blocker, a Kv1.3 inhibitor, or a peptide Z¹—X—Z² as described. The host cell may be capable of secreting the ion channel blocker, a Kv1.3 inhibitor, or a peptide Z¹—X—Z² as described.

The invention further provides a method of synthesising an ion channel blocker of the invention, the method comprising:
(a) synthesising the ion channel blocker by means of solid-phase or liquid-phase peptide synthesis methodology and recovering the peptide thus obtained;
(b) expressing the ion channel blocker from a nucleic acid construct that encodes the ion channel blocker and recovering the expression product; or
(c) expressing a precursor peptide from a nucleic acid construct that encodes the precursor peptide sequence, recovering the expression product, and modifying the precursor peptide to yield the ion channel blocker.

The invention further provides a pharmaceutical composition comprising an ion channel blocker of the invention or a pharmaceutically acceptable salt thereof, in admixture with a pharmaceutically acceptable carrier.

The invention further provides an ion channel blocker of the invention or a pharmaceutically acceptable salt thereof for use in a method of medical treatment.

The invention further provides an ion channel blocker of the invention or a pharmaceutically acceptable salt thereof for use in a method of inhibiting or reducing inflammation, especially in the treatment of an inflammatory condition or disorder, including autoimmune disorders.

An inflammatory condition or disorder may be any condition or disorder in which reduction of inflammation is desirable, e.g. where inflammation contributes to symptoms or pathogenesis.

Such conditions include autoimmune diseases, allergy and hypersensitivity, allograft rejection and graft versus host disease.

Specific conditions include hay fever, asthma, anaphylaxis, allergic rhinitis, urticaria, eczema, alopecia areata, dermatomyositis, inclusion body myositis, polymyositis, ankylosing spondylitis, vasculitis, arthritis (including rheumatoid arthritis, osteoarthritis, psoriatic arthritis), Sjogren's syndrome, systemic lupus erythematosus (SLE, or simply “lupus”), and uveitis, inflammatory fibrosis (e.g. scleroderma, lung fibrosis, cirrhosis), chronic obstructive pulmonary disease (COPD), hepatitis, chronic inflammatory demyelinating polyneuropathy, inflammatory bowel disease, colitis (including Crohn's disease and ulcerative colitis), erythema, thyroiditis, psoriasis, atopic dermatitis, allergic contact dermatitis, scleroderma, glomerulonephritis, inflammatory bone resorption, multiple sclerosis, type 1 diabetes, transplant rejection and graft-versus-host disease.

The invention further provides an ion channel blocker of the invention or a pharmaceutically acceptable salt thereof for use in a method of inhibiting weight gain, promoting weight loss, reducing excess body weight or treating obesity (e.g. by control of appetite, feeding, food intake, calorie intake, and/or energy expenditure), or in the treatment of obesity linked inflammation, obesity linked gallbladder disease or obesity induced sleep apnoea.

An effect on body weight may be therapeutic or cosmetic.

The invention further provides an ion channel blocker of the invention or a pharmaceutically acceptable salt thereof for use in treatment of a condition caused by or associated with impaired glucose control, such as metabolic syndrome, insulin resistance, glucose intolerance, pre-diabetes, increased fasting glucose or type 2 diabetes.

The invention further provides an ion channel blocker of the invention or a pharmaceutically acceptable salt thereof for use in treatment of a smooth muscle proliferative disorder such as restenosis, e.g. in patients following vascular surgery (e.g. angioplasty).

The invention further provides an ion channel blocker of the invention or a pharmaceutically acceptable salt thereof for use in the treatment of a neuroinflammatory or neurodegenerative disorder, such as Alzheimer's disease, multiple sclerosis (MS), Parkinson's disease or amyotrophic lateral sclerosis (ALS) (e.g. following viral infection).

The invention further provides an ion channel blocker of the invention or a pharmaceutically acceptable salt thereof for use in treatment of cancer, e.g. breast cancer, prostate cancer or lymphoma, such as non-Hodgkin lymphoma (NHL). NHL includes T-cell NHL and B-cell NHL.

Forms of B-cell NHL include diffuse large B-cell lymphoma, follicular lymphoma, Burkitt lymphoma, immunoblastic large cell lymphoma, precursor B-lymphoblastic lymphoma and mantle cell lymphoma. Forms of T-cell NHL include mycosis fungoides, anaplastic large cell lymphoma, peripheral T-cell lymphoma, precursor T-lymphoblastic lymphoma, and Sezary syndrome.

The invention further provides use of an ion channel blocker of the invention or a pharmaceutically acceptable salt thereof in the preparation of a medicament for inhibiting or reducing inflammation, especially in the treatment of an inflammatory condition or disorder, including autoimmune disorders. Further details of such conditions are set out above.

The invention further provides use of an ion channel blocker of the invention or a pharmaceutically acceptable salt thereof in the preparation of a medicament for use in inhibiting weight gain, promoting weight loss, reducing excess body weight or treating obesity (e.g. by control of appetite, feeding, food intake, calorie intake, and/or energy expenditure), or in the treatment of obesity linked inflammation, obesity linked gallbladder disease or obesity induced sleep apnoea.

The invention further provides use of an ion channel blocker of the invention or a pharmaceutically acceptable salt thereof in the preparation of a medicament for treatment of a condition caused by or associated with impaired glucose control, such as metabolic syndrome, insulin resistance, glucose intolerance, pre-diabetes, increased fasting glucose or type 2 diabetes.

The invention further provides use of an ion channel blocker of the invention or a pharmaceutically acceptable salt thereof in the preparation of a medicament for treatment of a smooth muscle proliferative disorder such as restenosis, e.g. in patients following vascular surgery (e.g. angioplasty).

The invention further provides use of an ion channel blocker of the invention or a pharmaceutically acceptable salt thereof in the preparation of a medicament for treatment of a neuroinflammatory or neurodegenerative disorder, such as Alzheimer's disease, multiple sclerosis (MS), Parkinson's disease or amyotrophic lateral sclerosis (ALS) (e.g. following viral infection).

The invention further provides use of an ion channel blocker of the invention or a pharmaceutically acceptable salt thereof in the preparation of a medicament for treatment of cancer, e.g. breast cancer, prostate cancer or lymphoma, such as non-Hodgkin lymphoma (NHL).

The invention further provides a method of inhibiting or reducing inflammation (especially in the treatment of an inflammatory condition or disorder, including autoimmune disorders) comprising administering an ion channel blocker of the invention or a pharmaceutically acceptable salt thereof to a subject in need thereof. Further details of such conditions are set out above.

The invention further provides a method of inhibiting weight gain, promoting weight loss, reducing excess body weight or treating obesity (e.g. by control of appetite, feeding, food intake, calorie intake, and/or energy expenditure), or treating obesity linked inflammation, obesity linked gallbladder disease or obesity induced sleep apnea, comprising administering an ion channel blocker of the invention or a pharmaceutically acceptable salt thereof to a subject in need thereof.

The invention further provides a method of treating a condition caused by or associated with impaired glucose control, such as metabolic syndrome, insulin resistance, glucose intolerance, pre-diabetes, increased fasting glucose or type 2 diabetes, comprising administering an ion channel blocker of the invention or a pharmaceutically acceptable salt thereof to a subject in need thereof.

The invention further provides a method of treating a smooth muscle proliferative disorder such as restenosis, e.g. in patients following vascular surgery (e.g. angioplasty), comprising administering an ion channel blocker of the invention or a pharmaceutically acceptable salt thereof to a subject in need thereof.

The invention further provides a method of treating a neuroinflammatory or neurodegenerative disorder, such as Alzheimer's disease, multiple sclerosis (MS), Parkinson's disease or amyotrophic lateral sclerosis (ALS) (e.g. following viral infection), comprising administering an ion channel blocker of the invention or a pharmaceutically acceptable salt thereof to a subject in need thereof.

The invention further provides a method of treating cancer, e.g. breast cancer, prostate cancer or lymphoma, such as non-Hodgkin lymphoma, comprising administering an ion channel blocker of the invention or a pharmaceutically acceptable salt thereof to a subject in need thereof.

### DETAILED DESCRIPTION OF THE INVENTION

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well known and commonly used in the art.

All patents, published patent applications and non-patent publications referred to in this application are specifically incorporated by reference herein. In case of conflict, the present specification, including its specific definitions, will control.

Each embodiment of the invention described herein may be taken alone or in combination with one or more other embodiments of the invention.

Unless specified otherwise, the following definitions are provided for specific terms which are used in the present written description.

Throughout this specification, the word “comprise”, and grammatical variants thereof, such as “comprises” or “comprising”, will be understood to imply the inclusion of a stated integer or component, or group of integers or components, but not the exclusion of any other integer or component, or group of integers or components.

The singular forms “a,” “an,” and “the” include the plurals unless the context clearly dictates otherwise.

The term “including” is used to mean “including but not limited to”. “Including” and “including but not limited to” may be used interchangeably.

The terms “patient”, “subject” and “individual” may be used interchangeably. A subject may be a mammal, including a human or a non-human mammal, such as a non-human primate (e.g. ape, Old World monkey or New World monkey), livestock animal (e.g. bovine or porcine), companion animal (e.g. canine or feline) or laboratory animal such as a rodent (e.g. mouse or rat).

Throughout the present description and claims the conventional three-letter and one-letter codes for naturally occurring amino acids are used, i.e.
A (Ala), G (Gly), L (Leu), I (lie), V (Val), F (Phe), W (Trp), S (Ser), T (Thr), Y (Tyr), N (Asn), Q (Gln), D (Asp), E (Glu), K (Lys), R (Arg), H (His), M (Met), C (Cys) and P (Pro);
as well as generally accepted codes for other α-amino acids, such as norleucine (Nle), sarcosine (Sar), α-aminoisobutyric acid (Aib), 2,3-diaminopropanoic acid (Dap), 2,4-diaminobutanoic acid (Dab), 2,5-diaminopentanoic acid (ornithine; Orn) alpha-aminobutyric acid (Abu, also known as homo-alanine), hK, hLys or homo-Lys (homo-lysine), hQ, hGln or homo-Gln (homo-glutamine, also known as 6-oxolysine, L-5-carbamoylnorvaline, 6-amino-6-oxonorleucine or 5-(aminocarbonyl)norvaline), F(4-F) (4-fluoro-phenylalanine), F(4-NH₂) (4-amino-phenylalanine), F(4-NO₂) (4-nitro-phenylalanine), F(4-CH₃) (4-methyl-phenylalanine).

The designation [2-Amino-5-carboxypentanoyl] indicates a peptide residue of 2-amino-5-carboxypentanoic acid:

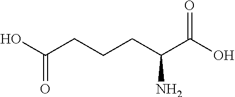 [SMILES: N[C@@H](CCCC(=O)O)C(=O)O]

which thus has a side chain similar to that of glutamic acid, but with an additional methylene group.

Such other α-amino acids may be shown in square brackets “[ ]” (e.g. “[Nle]”) when used in a general formula or sequence in the present specification, especially when the rest of the formula or sequence is shown using the single letter code. The 20 “naturally occurring” amino acids listed above are those which are encoded by the standard genetic code, and may also be referred to as “proteinogenic” amino acids.

Unless otherwise specified, amino acid residues in peptides of the invention are of the L-configuration. However, D-configuration amino acids may be incorporated. In the present context, an amino acid code written with a small letter represents the D-configuration of said amino acid, e.g. “k” represents the D-configuration of lysine (K).

A “H” (or “Hy-”) moiety at the N-terminus of the sequence in question indicates a hydrogen atom [i.e. R¹=hydrogen], corresponding to the presence of a free primary or secondary amino group at the N-terminus, while an “—OH” or an “—NH₂” moiety at the C-terminus of the sequence [i.e. R²═OH or NH₂] indicates the presence of a carboxy (COOH) group or an amido (CONH₂) group at the C-terminus of the molecule. A “CH₂OH” moiety at the C-terminus [i.e. R²═CH₂OH] indicates the presence of a hydroxyl group linked to an alkyl group at the C-terminus of the molecule. The CH₂OH moiety can be comprised in (4-amino-5-hydroxypentyl)guanidine or 4-amino-5-hydroxypentanamide. Designations of other R¹ and R² groups should be interpreted accordingly.

KV1.3 Blockers

The term Kv1.3 is used to refer to potassium voltage-gated channel subfamily A member 3, also referred to as KCNA3, HPCN3, HGK5, HuKIII and HLK3. “Subfamily A” may also be referred to as “shaker-related subfamily”. The human amino acid sequence is provided under UniProt accession number P22001, version P22001.3 (Q5VWN2).

The Kv1.3 channel is expressed on T and B lymphocytes and has been implicated in T cell activation. A number of groups are pursuing development of Kv1.3 blockers for the inhibition of immune responses as well as for various other indications. However, the Kv1.3 channel is part of a complex family of related ion channels, also including the Kv1.1, Kv1.2 and Kv1.6 channels, which have different physiological roles. Consequently it is desirable for Kv1.3 inhibitors to be as selective as possible for Kv1.3 in preference to other ion channels, especially other voltage-gated potassium channels, such as Kv1.1, Kv1.2, Kv1.4, Kv1.5, Kv1.6, Kv1.7 and Kv1.8.

The term “ion channel blocker” is used simply to denote a compound having inhibitor (or blocking) activity against an ion channel, i.e. capable of inhibiting or eliminating ion flow through the respective ion channel, typically by binding to the ion channel. Similarly, the terms “Kv1.3 inhibitor” and “Kv1.3 inhibitor component” refer to a peptide capable of inhibiting or eliminating ion flow through a Kv1.3 ion channel, typically by binding to the Kv1.3 channel. However, the terms “blocker” and “inhibitor” should not be taken to imply any particular mechanism of action, or any particular mode of interaction with the ion channel itself.

The ion channel blocker (and the Kv1.3 inhibitor component in isolation) of the invention has inhibitor or blocker activity at the Kv1.3 ion channel, i.e. it is capable of inhibiting ion flow through the Kv1.3 channel.

IC₅₀ values may be used as a measure of inhibitor (or blocker) activity or potency. An IC₅₀ value is a measure of the concentration of an inhibitor required to achieve half of that compound's maximal inhibition of ion channel activity in a given assay. A compound which has a lower IC₅₀ at a particular ion channel than a reference compound can be considered to be a more active inhibitor, or a more potent inhibitor, than the reference compound. The terms “activity” and “potency” are used interchangeably.

IC₅₀ values may be determined using any appropriate assay, such as fluorescence-based assays measuring ion flux (e.g. thallium ion flux) and patch clamp assays. They may be performed as described in the Examples below. Patch clamp assays may be preferred, e.g. using the QPatch® system.

The ion channel blocker (or Kv1.3 inhibitor) of the invention may have an IC₅₀ below 10 nM, but ideally the IC₅₀ is below 5 nM, below 2 nM or below 1 nM. In some cases it may be as low as 0.5 nM, 0.1 nM, or even lower.

Ion channel blockers of the invention may include the compounds 1-5, 7, 11-42, 44-48, 50-53, 55-60, 62-68, 70-73, 75-81, 83-88, 90-98, 101, 104-108, 116, 122, 129, 131, 132, 134 and 137 as described herein. These compounds are shown in Example 2 herein to have an IC₅₀ of 0.3 nM or less.

Ion channel blockers of the invention may include the compounds 7, 14-35, 37, 39, 40-48, 50-53, 55-60, 62-68, 70-73, 75-80, 83-97, 90, 91, 93-98, 101, 104-108, 116, 129 and 131 as described herein. These compounds are shown in Example 2 herein to have an IC₅₀ of 0.2 nM or less.

Ion channel blockers of the invention may include the compounds 7, 15-17, 19-28, 30-35, 37, 39-42, 44, 46-48, 51-53, 55-60, 62-67, 70, 71, 73, 75-80, 83-85, 87, 91, 93, 96-98, 104, 106, 107, 108 and 129 as described herein. These compounds are shown in Example 2 herein to have an IC₅₀ of 0.15 nM or less.

Ion channel blockers of the invention may include the compounds 1, 3, 17, 19, 21, 23, 26-33, 37, 41-43, 46-49, 52, 62, 63, 66-68, 70-73, 75, 76, 78, 82-84, 87, 91, 94, 97-102, 105-108, 115 and 116 as described herein. These compounds are shown in Example 5 herein to have an IC₅₀ of 1 nM or less for inhibiting activation of T cells in rat whole blood.

The ion channel blockers of the invention are selective for Kv1.3. In an embodiment the ion channel blockers of the invention are selective over Kv1.1, Kv1.2, Kv1.4, Kv1.5, Kv1.6, Kv1.7 and Kv1.8. In particular, the ion channel blockers of the invention are selective for Kv1.3 over one or more of Kv1.1, Kv1.2 and Kv1.6.

For example, they may be:
selective for Kv1.3 over Kv1.1;
selective for Kv1.3 over Kv1.2;
selective for Kv1.3 over Kv1.6;
selective for Kv1.3 over Kv1.1 and Kv1.2;
selective for Kv1.3 over Kv1.1 and Kv1.6;
selective for Kv1.3 over Kv1.2 and Kv1.6; or
selective for Kv1.3 over Kv1.1, Kv1.2 and Kv1.6.

Typically, the ion channel blockers are selective for Kv1.3 over Kv1.1.

They may additionally be selective for Kv1.3 over Kv1.2 and/or Kv1.6.

By “selective” in this context is meant that the ion channel blockers have higher inhibitor activity against Kv1.3 than against the respective ones of Kv1.1, Kv1.2 and Kv1.6. Thus, their IC₅₀ against Kv1.3 is typically lower than against the respective other ion channel or channels.

Selectivity for Kv1.3 over another ion channel X may therefore be expressed as a ratio of the respective IC₅₀ values, e.g. as IC₅₀[X]/IC₅₀[Kv1.3].

The ion channel blockers of the invention may therefore have a selectivity for Kv1.3 over Kv1.1 of at least 10, at least 100, at least 1000, or at least 10000, and may be up to 100000 or even higher. Typically, they have a selectivity for Kv1.3 over Kv1.1 of at least 100, or at least 1000.

Ion channel blockers of the invention may include compounds 1-3, 5, 8, 12, 16-23, 25-31, 34-37, 40-42, 45-49, 52-54, 56, 58, 62, 63, 70, 71, 76, 79, 83, 94-98, 101, 103-108 and 117 as described herein. These compounds are shown in Example 3 herein to have selectivity for Kv1.3 over Kv1.1 of at least 1000.

Ion channel blockers of the invention may include compounds 3, 12, 16, 18-22, 25-28, 30, 31, 35, 37, 40-42, 52, 53, 62, 70, 71, 76, 79, 94, 95, 98 and 105 as described herein. These compounds are shown in Example 3 herein to have selectivity for Kv1.3 over Kv1.1 of at least 10000.

The ion channel blockers of the invention may therefore have a selectivity for Kv1.3 over Kv1.2 of at least 10, at least 100, at least 1000, or at least 10000, and may be up to 100000 or even higher. Typically, they have a selectivity for Kv1.3 over Kv1.2 of at least 10, and preferably at least 50 or at least 100 or at least 1000.

Ion channel blockers of the invention may include compounds 1-3, 5, 8, 16-23, 25-31, 34-37, 40-42, 45-49, 52-54, 56, 58, 62, 63, 70, 71, 76, 79, 83, 94-98, 101, 103-108 and 117 as described herein. These compounds are shown in Example 3 herein to have selectivity for Kv1.3 over Kv1.2 of at least 50.

Ion channel blockers of the invention may include compounds 1, 3, 5, 8, 12, 18, 25, 29, 30, 36, 37, 41, 46-48, 52, 62, 70, 71, 79, 83, 94-98, 101, 103-106 and 117 as described herein. These compounds are shown in Example 3 herein to have selectivity for Kv1.3 over Kv1.2 of at least 700.

Ion channel blockers of the invention may include compounds 1, 3, 12, 18, 29, 30, 36, 37, 41, 47, 48, 62, 70, 71, 79, 94-98, 101 and 103-105 as described herein. These compounds are shown in Example 3 herein to have selectivity for Kv1.3 over Kv1.2 of at least 1000.

The ion channel blockers of the invention may therefore have a selectivity for Kv1.3 over Kv1.6 of at least 10, at least 100, at least 1000, or at least 10000, and may be up to 100000 or even higher. Typically, they have a selectivity for Kv1.3 over Kv1.6 of at least 100, or at least 400, or at least 1000.

Ion channel blockers of the invention may include compounds 1, 2, 3, 5, 8, 12, 16-23, 25-31, 34-37, 40-42, 45-49, 52-54, 56, 58, 62, 63, 70, 71, 76, 79, 83, 94-98, 101, 103-108 and 117 as described herein. These compounds are shown in Example 3 herein to have selectivity for Kv1.3 over Kv1.6 of at least 400.

Ion channel blockers of the invention may include compounds 3, 12, 16, 18, 20, 22, 26, 30, 31, 37, 41, 52, 53, 70, 71, 76, 79, 94, 95, 98 and 105 as described herein. These compounds are shown in Example 3 herein to have selectivity for Kv1.3 over Kv1.6 of at least 10000.

The ion channel blockers of the invention may have greater selectivity than known ion channel blockers such as ShK, Mokatoxin (Moka1), Vm24, Odk2 or Osk1. Thus the ion channel blockers of the invention may have higher selectivity for Kv1.3 over ion channel X,
i.e. IC₅₀[X]/IC₅₀[Kv1.3],

which is greater than the selectivity of the comparison molecule. The selectivity of the two ion channel blockers will be determined under the same conditions for each ion channel to enable direct comparison. As mentioned above, any appropriate assays may be used, such as fluorescence-based ion flux assays and patch clamp assays.

The ion channel blockers of the invention may have lower absolute inhibitor activity (i.e. higher IC₅₀) than known ion channel blockers (such as Odk2 or Osk1) at any or all of Kv1.1, Kv1.2 and/or Kv1.6. However, it may be acceptable for them to have higher absolute inhibitor activity at any or all of these ion channels, as long as their selectivity for Kv1.3 is higher than that of the comparison compound. Typically, though, the compounds of the invention combine high specificity for Kv1.3 with high potency.

Synthesis and Recombinant Expression

The ion channel blockers described herein may be synthesised by means of solid-phase or liquid-phase peptide synthesis methodology. In this context, reference may be made to WO 98/11125 and, among many others, Fields, G. B. et al., 2002, “Principles and practice of solid-phase peptide synthesis”. In: Synthetic Peptides (2nd Edition), and the Examples herein.

Alternatively, the ion channel blockers described herein may be synthesised by recombinant techniques, or by a combination of recombinant techniques and peptide chemistry.

For example, an ion channel blocker peptide may be synthesised by a method which comprises
(a) synthesising the peptide by means of solid-phase or liquid-phase peptide synthesis methodology and recovering the peptide thus obtained;
(b) expressing the peptide from a nucleic acid construct that encodes the peptide and recovering the expression product; or
(c) expressing a precursor peptide from a nucleic acid construct that encodes the precursor peptide sequence, recovering the expression product, and modifying the precursor peptide to yield an ion channel blocker of the invention.

The precursor peptide may be modified by introduction of one or more non-proteinogenic amino acids (e.g. Nle), introduction of the appropriate terminal groups R¹ and R², etc.

Expression of the peptide or precursor peptide from a nucleic acid encoding the peptide or precursor peptide may be performed in a cell or a cell-free expression system comprising such a nucleic acid. Such expression typically requires that the peptide or precursor peptide is composed entirely of proteinogenic amino acids (i.e. the 20 amino acids encoded by the standard genetic code.)

For recombinant expression, the nucleic acid fragments encoding the precursor peptide will normally be inserted in suitable vectors to form cloning or expression vectors. The vectors can, depending on purpose and type of application, be in the form of plasmids, phages, cosmids, mini-chromosomes, or virus, but also naked DNA which is only expressed transiently in certain cells is an important vector. Preferred cloning and expression vectors (plasmid vectors) are capable of autonomous replication, thereby enabling high copy-numbers for the purposes of high-level expression or high-level replication for subsequent cloning.

In general outline, an expression vector comprises the following features in the 5′→3′ direction and in operable linkage: a promoter for driving expression of the nucleic acid fragment, optionally a nucleic acid sequence encoding a leader peptide enabling secretion (to the extracellular phase or, where applicable, into the periplasm), the nucleic acid fragment encoding the precursor peptide, and optionally a nucleic acid sequence encoding a terminator. They may comprise additional features such as selectable markers and origins of replication. When operating with expression vectors in producer strains or cell lines it may be preferred that the vector is capable of integrating into the host cell genome. The skilled person is very familiar with suitable vectors and is able to design one according to their specific requirements.

The vectors of the invention are used to transform host cells to produce the peptide or precursor peptide. Such transformed cells can be cultured cells or cell lines used for propagation of the nucleic acid fragments and vectors, and/or used for recombinant production of the precursor peptides.

Preferred transformed cells are micro-organisms such as bacteria [such as the species Escherichia (e.g. E. coli), Bacillus (e.g. Bacillus subtilis), Salmonella, or Mycobacterium (preferably non-pathogenic, e.g. M. bovis BCG), yeasts (e.g., Saccharomyces cerevisiae and Pichia pastoris), and protozoans. Alternatively, the transformed cells may be derived from a multicellular organism, i.e. it may be fungal cell, an insect cell, an algal cell, a plant cell, or an animal cell such as a mammalian cell. For the purposes of cloning and/or optimised expression it is preferred that the transformed cell is capable of replicating the nucleic acid fragment of the invention. Cells expressing the nucleic fragment can be used for small-scale or large-scale preparation of the peptides of the invention.

When producing the peptide or precursor peptide by means of transformed cells, it is convenient, although far from essential, that the expression product is secreted into the culture medium.

Therapeutic Indications

As discussed above, blockers of Kv1.3 have been shown to inhibit proliferation of activated T cells and to have a beneficial effect in various experimental models of disease. Without wishing to be bound by theory, it is believed that cellular efflux of potassium via the Kv1.3 channel is required to sustain calcium influx required for T-cell activation.

Kv1.3 is overexpressed in Gad5/insulin-specific T cells from patients with new onset type 1 diabetes, in myelin-specific T cells from MS patients and in T cells from the synovium of rheumatoid arthritis patients (Beeton et al., Proc Natl Acad Sci USA 103:17414-9, 2006), in breast cancer specimens (Abdul et al., Anticancer Res 23:3347, 2003) and prostate cancer cell lines (Fraser et al., Pflugers Arch 446:559, 2003).

Positive outcomes in animal models with Kv1.3 blockers have been described in hypersensitivity models to ovalbumin and tetanus toxoid (Beeton et al., Mol Pharmacol 67:1369, 2005; Koo et al., Clin Immunol 197:99, 1999), models for multiple sclerosis such as rat adoptive-transfer experimental autoimmune encephalomyelitis (AT-EAE) model (Beeton et al., Proc Natl Acad Sci USA 103:17414-9, 2006), inflammatory bone resorption model (Valverde et al., J Bone Mineral Res 19:155, 2004), models for arthritis (Beeton et al., Proc Natl Acad Sci 103: 17414, 2006; Tarcha et al., J. Pharmacol. Exp. Ther. 342: 642, 2012) and obesity, diabetes and metabolic disorders (Xu et al., Hum Mol Genet 12:551, 2003; Xu et al., Proc Natl Acad Sci 101: 3112, 2004).

Topical application of Kv1.3 blockers has been proposed for the treatment of skin and mucosal inflammation.

Thus, the ion channel blockers described in this specification have considerable potential for use in inhibiting or reducing inflammation, especially in the treatment of an inflammatory condition or disorder, including autoimmune disorders.

An inflammatory condition or disorder may be any condition or disorder in which reduction of inflammation is desirable, e.g. where inflammation contributes to symptoms or pathogenesis.

Such conditions include autoimmune disorders, allergy and hypersensitivity, allograft rejection and graft versus host disease.

More specifically, conditions include hay fever, asthma, anaphylaxis, allergic rhinitis, urticaria, eczema, alopecia areata, dermatomyositis, inclusion body myositis, polymyositis, ankylosing spondylitis, vasculitis, arthritis (including rheumatoid arthritis, osteoarthritis, psoriatic arthritis), Sjogren's syndrome, systemic lupus erythematosus (SLE, or simply “lupus”), and uveitis, inflammatory fibrosis (e.g. scleroderma, lung fibrosis, cirrhosis), chronic obstructive pulmonary disease (COPD), hepatitis, chronic inflammatory demyelinating polyneuropathy, inflammatory bowel disease, colitis (including Crohn's disease and ulcerative colitis), erythema, thyroiditis, psoriasis, atopic dermatitis, allergic contact dermatitis, scleroderma, glomerulonephritis, inflammatory bone resorption, multiple sclerosis, type 1 diabetes, transplant rejection and graft-versus-host disease.

Blockers of Kv1.3 may also have beneficial metabolic effects, e.g. in relation to energy homeostasis, body weight regulation, and glucose control.

The ion channel blockers described here may therefore be used for inhibiting weight gain, promoting weight loss, reducing excess body weight or treating obesity (e.g. by control of appetite, feeding, food intake, calorie intake, and/or energy expenditure), as well as in the treatment of associated disorders and health conditions including obesity linked inflammation, obesity linked gallbladder disease and obesity induced sleep apnoea.

An effect on body weight may be therapeutic or cosmetic.

The ion channel blockers may also be used for the treatment of conditions caused by or associated with impaired glucose control, including metabolic syndrome, insulin resistance, glucose intolerance, pre-diabetes, increased fasting glucose and type 2 diabetes. Some of these conditions can be associated with obesity. Their effects on these conditions may be mediated in whole or in part via an effect on body weight, or may be independent thereof.

Kv1.3 is also expressed in proliferating human and mouse smooth muscle cells. Blockers of Kv1.3 may be effective in smooth muscle proliferative disorders such as restenosis, e.g. in patients following vascular surgery (e.g. angioplasty).

Further evidence suggests that Kv1.3 channels are involved in the activation and/or proliferation of many types of cells, including tumor cells (Bielanska et al., Curr. Cancer Drug Targets 9:904-14, 2009), microglia (Khanna et al., Am. J. Physiol. Cell Physiol. 280: C796-806, 2001) and differentiation of neuronal progenitor cells (Wang et al., J. Neurosci. 30:5020-7, 2010). Kv1.3 blockers may therefore be beneficial in the treatment of neuroinflammatory and neurodegenerative disorders such as Alzheimer's disease, multiple sclerosis (MS), Parkinson's disease and amyotrophic lateral sclerosis (ALS) (e.g. following viral infections), and cancers including breast cancer, prostate cancer, and lymphoma, such as non-Hodgkin lymphoma (NHL). Non-Hodgkin lymphomas include T-cell NHL and B-cell NHL. Forms of B-cell NHL include diffuse large B-cell lymphoma, follicular lymphoma, Burkitt lymphoma, immunoblastic large cell lymphoma, precursor B-lymphoblastic lymphoma and mantle cell lymphoma. Forms of T-cell NHL include mycosis fungoides, anaplastic large cell lymphoma, peripheral T-cell lymphoma, precursor T-lymphoblastic lymphoma and Sezary syndrome.

Pharmaceutical Compositions and Administration

An aspect of the present invention relates to a composition comprising an ion channel blocker of the invention, or a salt thereof, together with a carrier, excipient or vehicle. In certain embodiments, the composition is a pharmaceutical composition, any salt is a pharmaceutically acceptable salt, and the carrier is a pharmaceutically acceptable carrier, excipient or vehicle.

Accordingly, the compounds of the present invention, or salts thereof, especially pharmaceutically acceptable salts thereof, may be formulated as compositions or pharmaceutical compositions prepared for storage or administration, and which comprise a therapeutically effective amount of a compound of the invention, or a salt thereof.

Suitable salts formed with bases include metal salts, such as alkali metal or alkaline earth metal salts, for example sodium, potassium or magnesium salts; ammonia salts and organic amine salts, such as those formed with morpholine, thiomorpholine, piperidine, pyrrolidine, a lower mono-, di- or tri-alkylamine (e.g., ethyl-tert-butyl-, diethyl-, diisopropyl-, triethyl-, tributyl- or dimethylpropylamine), or a lower mono-, di- or tri-(hydroxyalkyl)amine (e.g., mono-, di- or triethanolamine). Internal salts may also be formed. Similarly, when a compound of the present invention contains a basic moiety, salts can be formed using organic or inorganic acids. For example, salts can be formed from the following acids: formic, acetic, propionic, butyric, valeric, caproic, oxalic, lactic, citric, tartaric, succinic, fumaric, maleic, malonic, mandelic, malic, phthalic, hydrochloric, hydrobromic, phosphoric, nitric, sulphuric, benzoic, carbonic, uric, methanesulphonic, naphthalenesulphonic, benzenesulphonic, toluenesulphonic, p-toluenesulphonic (i.e. 4-methylbenzene-sulphonic), camphorsulphonic, 2-aminoethanesulphonic, aminomethylphosphonic and trifluoromethanesulphonic acid (the latter also being denoted triflic acid), as well as other known pharmaceutically acceptable acids. Amino acid addition salts can also be formed with amino acids, such as lysine, glycine, or phenylalanine.

In some embodiments, a pharmaceutical composition of the invention is one wherein the compound is in the form of a pharmaceutically acceptable acid addition salt.

As will be apparent to one skilled in the medical art, a “therapeutically effective amount” of a compound or pharmaceutical composition of the present invention will vary depending upon, inter alia, the age, weight and/or gender of the subject (patient) to be treated. Other factors that may be of relevance include the physical characteristics of the specific patient under consideration, the patient's diet, the nature of any concurrent medication, the particular compound(s) employed, the particular mode of administration, the desired pharmacological effect(s) and the particular therapeutic indication. Because these factors and their relationship in determining this amount are well known in the medical arts, the determination of therapeutically effective dosage levels to achieve the desired therapeutic effect will be within the ambit of the skilled person.

As used herein, the term “a therapeutically effective amount” refers to an amount which reduces symptoms of a given condition or pathology, and preferably which normalizes physiological responses in an individual with that condition or pathology. Reduction of symptoms or normalization of physiological responses can be determined using methods routine in the art and may vary with a given condition or pathology. In one aspect, a therapeutically effective amount of a compound of the invention, or a pharmaceutical composition, is an amount which restores a measurable physiological parameter to substantially the same value (preferably to within 30%, more preferably to within 20%, and still more preferably to within 10% of the value) of the parameter in an individual without the condition or pathology in question.

In one embodiment of the invention, administration of a compound or pharmaceutical composition of the present invention is commenced at lower dosage levels, with dosage levels being increased until the desired effect of preventing/treating the relevant medical indication is achieved. This would define a therapeutically effective amount. For the compounds of the present invention, alone or as part of a pharmaceutical composition, such human doses of the active compound may be between about 0.01 pmol/kg and 500 μmol/kg body weight, between about 0.01 pmol/kg and 300 pmol/kg body weight, between 0.01 pmol/kg and 100 μmol/kg body weight, between 0.1 pmol/kg and 50 μmol/kg body weight, between 1 μmol/kg and 10 μmol/kg body weight, between 5 pmol/kg and 5 μmol/kg body weight, between 10 pmol/kg and 1 μmol/kg body weight, between 50 pmol/kg and 0.1 μmol/kg body weight, between 100 pmol/kg and 0.01 μmol/kg body weight, between 0.001 μmol/kg and 0.5 μmol/kg body weight, between 0.05 μmol/kg and 0.1 μmol/kg body weight.

An effective dosage and treatment protocol may be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject. Such considerations are known to the skilled person.

### EXAMPLES

### Example 1: General Peptide Synthesis

List of abbreviations and suppliers are provided in the table below

List of abbreviations and suppliers

| | Abbreviation | Name | Brand/Supplier |
|---|---|---|---|
| Resins | | | |
| | | TentaGel ™ PHB AA(Proct)- | Rapp Polymere |
| | | Fmoc | |
| | | TentaGel ™ SRAM | Rapp Polymere |
| Amino | | | |
| acids | | | |
| | | Pseudoprolines (E.g. QT, AT, | Jupiter Bioscience |
| | | FS) | Ltd. |
| | | Fmoc-L-AA-OH | Senn Chemicals |
| | | | AG |
| Coupling | | | |
| reagents | | | |
| | COMU | (1-Cyano-2-ethoxy-2- | Watson |
| | | oxoethylidenaminooxy)dimethylamino- | International Ltd. |
| | | morpholino-carbenium | |
| | | hexafluorophosphate | |
| | DIC | Diisopropylcarbodiimide | Fluka/Sigma |
| | | | Aldrich Co. |
| | HATU | N-[(dimethylamino)-1H-1,2,3- | ChemPep Inc. |
| | | triazol[4,5-b]pyridine-1- | |
| | | ylmethylene]-N- | |
| | | methylmethanaminium | |
| | | hexafluorophosphate N-oxide | |
| | HOBt | Hydroxybenzotriazole | Sigma-Aldrich Co. |
| Solvents | | | |
| reagents | | | |
| | Boc₂O | Di-tert-butyl pyrocarbonate | Advanced |
| | | | ChemTech |
| | DCM | Dichloromethane | Prolabo (VWR) |
| | DIPEA | Diisopropylethylamine | Fluka/Sigma |
| | | | Aldrich Co. |
| | DMF | N,N-dimethylformamide | Taminco |
| | DODT | 3,6-dioxa-1,8-octanedithiol | Sigma-Aldrich Co. |
| | Et₂O | Diethyl ether | Prolabo (VWR) |
| | EtOH | Ethanol | CCS Healthcare |
| | | | AB |
| | | Formic acid (HPLC) | Sigma-Aldrich Co. |
| | H₂O | Water, Milli-Q water | Millipore |
| | MeCN | Acetonitrile (HPLC) | Sigma-Aldrich Co. |
| | NMP | N-methylpyrrolidone | Sigma-Aldrich Co. |
| | | Piperidine | Jubliant Life |
| | | | Sciences Ltd. |
| | TFA | Trifluoroacetic acid (HPLC) | Chemicals Raw |
| | | | Materials Ltd. |
| | TIS | Triisopropylsilane | Sigma-Aldrich Co. |
| | MeOH | Methanol | Sigma-Aldrich Co. |

Apparatus and Synthetic Strategy

Peptides were synthesized batchwise on a peptide synthesiser, such as a CEM Liberty Peptide Synthesizer or a Symphony X Synthesizer, according to solid phase peptide synthetic procedures using 9-fluorenylmethyloxycarbonyl (Fmoc) as N-α-amino protecting group and suitable common protection groups for side-chain functionalities.

As polymeric support based resins, such as e.g. TentaGel™, was used. The synthesizer was loaded with resin that prior to usage was swelled in DMF.

Coupling

CEM Liberty Peptide Synthesizer

A solution of Fmoc-protected amino acid (4 equiv.) was added to the resin together with a coupling reagent solution (4 equiv.) and a solution of base (8 equiv.). The mixture was either heated by the microwave unit to 70-75° C. and coupled for 5 minutes or coupled with no heat for 60 minutes. During the coupling nitrogen was bubbled through the mixture.

Symphony X Synthesizer

The coupling solutions were transferred to the reaction vessels in the following order: amino acid (4 equiv.), HATU (4 equiv.) and DIPEA (8 equiv.). The coupling time was 10 min at room temperature (RT) unless otherwise stated. The resin was washed with DMF (5×0.5 min). In case of repeated couplings the coupling time was in all cases 45 min at RT.

Deprotection

CEM Liberty Peptide Synthesizer

The Fmoc group was deprotected using piperidine in DMF or other suitable solvents. The deprotection solution was added to the reaction vessel and the mixture was heated for 30 sec. reaching approx. 40° C. The reaction vessel was drained and fresh deprotection solution was added and subsequently heated to 70-75° C. for 3 min. After draining the reaction vessel the resin was washed with DMF or other suitable solvents.

Symphony X Synthesizer

Fmoc deprotection was performed for 2.5 minutes using 40% piperidine in DMF and repeated using the same conditions. The resin was washed with DMF (5×0.5 min).

Cleavage

The dried peptide resin was treated with TFA and suitable scavengers for approximately 2 hours. The volume of the filtrate was reduced and the crude peptide was precipitated after addition of diethylether. The crude peptide precipitate was washed several times with diethylether and finally dried.

HPLC Purification of the Crude Peptide

The crude peptide was purified by preparative reverse phase HPLC using a conventional HPLC apparatus, such as a Gilson GX-281 with 331/332 pump combination’, for binary gradient application equipped with a column, such as 5×25 cm Gemini NX 5u C18 110A column, and a fraction collector using a flow 20-40 ml/min with a suitable gradient of buffer A (0.1% Formic acid, aq.) or A (0.1% TFA, aq.) and buffer B (0.1% Formic acid, 90% MeCN, aq.) or B (0.1% TFA, 90% MeCN, aq.). Fractions were analyzed by analytical HPLC and MS and selected fractions were pooled and lyophilized. The final product was characterized by HPLC and MS.

Disulphide Formation

The crude or partially purified linear peptide with six cysteines was dissolved in a buffer such as sodium hydrogen carbonate (NaHCO₃) or ammonium acetate (NH₄Ac) to give a final concentration of approximate 0.1 mg/ml or 25 μM. The pH of the buffer was adjusted to pH 8.0 and the solution was stirred at room temperature under magnetic stirring and open access to the atmosphere. The progress of the reaction was determined by HPLC and was usually evaluated to be complete overnight. The solution was quenched by reducing the pH of the solution by an organic acid such as acetic acid or trifluoroacetic acid (pH<4). The solution was filtered and loaded directly on a prep-HPLC column for purification.

Analytical HPLC

Final purities were determined by analytic HPLC (Agilent 1100/1200 series) equipped with auto sampler, degasser, 20 μl flow cell and Chromeleon software. The HPLC was operated with a flow of 1.2 ml/min at 40° C. using an analytical column, such as Kinetex 2.6 μm XB-C18 100A 100×4.6 mm column. The compound was detected and quantified at 215 nm. Buffers A (0.1% TFA, aq.) and buffer B (0.1% TFA, 90% MeCN, aq.).

Mass Spectroscopy

Final MS analysis was performed on a conventional mass spectrometer, e.g. Waters Xevo G2 Tof, equipped with electrospray detector with lock-mass calibration and MassLynx software. It was operated in positive mode using direct injection and a cone voltage of 15V (1 TOF), 30 V (2 TOF) or 45 V (3 TOF) as specified on the chromatogram. Precision was 5 ppm with a typical resolution of 15,000-20,000.

Compounds synthesized are shown in Table 1.

TABLE 1

| Cmpnd | |
|---|---|
| No. | Sequence |
| 1 | H-QMDMRCSASVECKQKCLKAIGSIFGKCMNKKCKCYPR-OH |
| 2 | H-QMDMRCSASVECKQKCLKAIGRGFGKCMNKKCKCYPR-OH |
| 3 | H-NMDMRCSASVECKQKCLKAIGSIFGKCMNKKCKCYPR-OH |
| 4 | H-NMDMRCSASVECKQKCLKAIGRGFGKCMNKKCKCYPR-OH |
| 5 | H-MDMRCSASVECKQKCLKAIGSIFGKCMNKKCKCYPR-OH |
| 6 | H-DMRCSASVECKQKCLKAIGSIFGKCMNKKCKCYPR-OH |
| 7 | H-NMDMRCSASVECKQKCLKAIGSIFGKCMNKKCKCYPR-NH₂ |
| 8 | H-NIDMRCSASVECKQKCLKAIGSIFGKCMNKKCKCYPR-OH |
| 9 | H-NMDVRCSASVECKQKCLKAIGSIFGKCMNKKCKCYPR-OH |
| 10 | H-NMDMRCSASVECKQKCKDAIGSIFGKCMNKKCKCYPR-OH |
| 11 | H-GGNMDMRCSASVECKQKCLKAIGSIFGKCMNKKCKCYPR-OH |
| 12 | H-NMEMRCSASVECKQKCLKAIGSIFGKCMNKKCKCYPR-OH |
| 13 | H-SGNMDMRCSASVECKQKCLKAIGSIFGKCMNKKCKCYPR-OH |
| 14 | H-N[Nle]DMRCSASVECKQKCLKAIGSIFGKCMNKKCKCYPR-NH₂ |
| 15 | H-NMD[Nle]RCSASVECKQKCLKAIGSIFGKCMNKKCKCYPR-NH₂ |
| 16 | H-NMDMRCSASVECKQKCLKAIGSIFGKC[Nle]NKKCKCYPR-NH₂ |
| 17 | H-NMDMRCSASVECKVKCLKAIGSIFGKCMNKKCKCYPR-NH₂ |
| 18 | H-NMDMRCSASVECKQLCLKAIGSIFGKCMNKKCKCYPR-NH₂ |
| 19 | H-NMDMRCSASVECKQKCKKAIGSIFGKCMNKKCKCYPR-NH₂ |
| 20 | H-NMDMRCSASVECKQKCLDAIGSIFGKCMNKKCKCYPR-NH₂ |
| 21 | H-NMDMRCSASVECKQKCLKAIRSIFGKCMNKKCKCYPR-NH₂ |
| 22 | H-NMDMRCSASVECKQKCLKAIESIFGKCMNKKCKCYPR-NH₂ |
| 23 | H-NMDMRCSASVECKQKCLKAIGSIFGKCMNKKCKCYPRRRTA-NH₂ |
| 24 | H-NMDMRCSASVECKQKCLKAIGSIFGKCMNKKCKCYPRHRRK-NH₂ |
| 25 | H-NMDMRCKASVECKQKCLKAIGSIFGKCMNKKCKCYPR-NH₂ |
| 26 | H-NMDMRCSISVECKQKCLKAIGSIFGKCMNKKCKCYPR-NH₂ |
| 27 | H-NMDMRCSASRECKQKCLKAIGSIFGKCMNKKCKCYPR-NH₂ |
| 28 | H-NMDMRCSASVQCKQKCLKAIGSIFGKCMNKKCKCYPR-NH₂ |
| 29 | H-NMDMRCSASVECLQKCLKAIGSIFGKCMNKKCKCYPR-NH₂ |
| 30 | H-NMDMRCSASVECAQKCLKAIGSIFGKCMNKKCKCYPR-NH₂ |
| 31 | H-NMDMRCSASVECKEKCLKAIGSIFGKCMNKKCKCYPR-NH₂ |
| 32 | H-NMDMRCSASVECKLKCLKAIGSIFGKCMNKKCKCYPR-NH₂ |
| 33 | H-NMDMRCSASVECKQKCLKAIHSIFGKCMNKKCKCYPR-NH₂ |
| 34 | H-NMDMRCSASVECKQKCLKAIGSKFGKCMNKKCKCYPR-NH₂ |
| 35 | H-NMDMRCSASVECKQKCLKAIGSRFGKCMNKKCKCYPR-NH₂ |
| 36 | H-NMDMRCSASVECKQKCLKAIGSIFGKCMNGKCKCYPR-NH₂ |
| 37 | H-NMDMRCSASVECKQKCLKAIGSIFGKCMNKKCHCYPR-NH₂ |
| 38 | H-NMDMRCSASVECKQKCLKAIGSIFGKCMNKKCVCYPR-NH₂ |
| 39 | H-N[Nle]D[Nle]RCSASVECKQKCLKAIGSIFGKCMNKKCKCYPR-NH₂ |
| 40 | H-N[Nle]D[Nle]RCSASVECKQKCLKAIGSIFGKC[Nle]NKKCKCYPR-NH₂ |
| 41 | H-P[Nle]D[Nle]RCSASVECKQKCLKAIGSIFGKC[Nle]NKKCKCYPR-NH₂ |
| 42 | H-N[Nle]D[Nle]RCRASVECKQKCLKAIGSIFGKC[Nle]NKKCKCYPR-NH₂ |
| 43 | H-N[Nle]D[Nle]RCSASVECEQKCLKAIGSIFGKC[Nle]NKKCKCYPR-NH₂ |
| 44 | H-N[Nle]D[Nle]RCSASVECQQKCLKAIGSIFGKC[Nle]NKKCKCYPR-NH₂ |
| 45 | H-N[Nle]D[Nle]RCSASVECKKKCLKAIGSIFGKC[Nle]NKKCKCYPR-NH₂ |
| 46 | H-N[Nle]S[Nle]RCSASVECKQKCLKAIGSIFGKC[Nle]NKKCKCYPR-NH₂ |
| 47 | H-N[Nle]D[Nle]RCSHSVECKQKCLKAIGSIFGKC[Nle]NKKCKCYPR-NH₂ |
| 48 | H-N[Nle]D[Nle]RCSASVECKQSCLKAIGSIFGKC[Nle]NKKCKCYPR-NH₂ |
| 49 | H-N[Nle]D[Nle]RCSASVECKQKCKAIGSIFGKC[Nle]NKKCKCYPR-NH₂ |
| 50 | H-NMDMRCSASVECKQKCYKAIGSIFGKCMNKKCKCYPR-NH₂ |
| 51 | H-NMDMRCSASVECKQKCRKAIGSIFGKCMNKKCKCYPR-NH₂ |
| 52 | H-NMDMRCSASVECKQKCLAAIGSIFGKCMNKKCKCYPR-NH₂ |
| 53 | H-NMDMRCSASVECKQKCLYAIGSIFGKCMNKKCKCYPR-NH₂ |
| 54 | H-NMDMRCSASVECKQKCLAIGSIFGKCMNKKCKCYPR-NH₂ |
| 55 | H-NMDMRCSASVECKQKCLKYIGSIFGKCMNKKCKCYPR-NH₂ |
| 56 | H-NMDMRCSASVECKQKCLKRIGSIFGKCMNKKCKCYPR-NH₂ |
| 57 | H-NMDMRCSASVECKQKCLKIGSIFGKCMNKKCKCYPR-NH₂ |
| 58 | H-NMDMRCSASVECKQKCLKAYGSIFGKCMNKKCKCYPR-NH₂ |
| 59 | H-NMDMRCSASVECKQKCLKAEGSIFGKCMNKKCKCYPR-NH₂ |
| 60 | H-NMDMRCSASVECKQKCLKARGSIFGKCMNKKCKCYPR-NH₂ |
| 61 | H-NMDMRCSASVECKQKCLKAGSIFGKCMNKKCKCYPR-NH₂ |
| 62 | H-N[Nle]D[Nle]RCSASVECKQKCLKAIGSPFGKC[Nle]NKKCKCYPR-NH₂ |
| 63 | H-N[Nle]D[Nle]RCSASKECKQKCLKAIGSIFGKC[Nle]NKKCKCYPR-NH₂ |
| 64 | H-H[Nle]D[Nle]RCSASVECKQKCLKAIGSIFGKC[Nle]NKKCKCYPR-NH₂ |
| 65 | H-Y[Nle]D[Nle]RCSASVECKQKCLKAIGSIFGKC[Nle]NKKCKCYPR-NH₂ |
| 66 | H-S[Nle]D[Nle]RCSASVECKQKCLKAIGSIFGKC[Nle]NKKCKCYPR-NH₂ |
| 67 | H-V[Nle]D[Nle]RCSASVECKQKCLKAIGSIFGKC[Nle]NKKCKCYPR-NH₂ |
| 68 | H-S[Nle]D[Nle]RCSA[Abu]VECKQKCLKAIGSIFGKCMNKKCKCYPR-NH₂ |
| 69 | H-S[Nle]D[Nle]RCSASVECKQKCLKAIGSIFG[homo- |
| | Lys]CMNKKCKCYPR-NH₂ |
| 70 | H-S[Nle]D[Nle]RCSASVECKQKCLKAIGSIFGKCMNKKCKC[F(4- |
| | NH₂)]PR-NH₂ |
| 71 | H-S[Nle]D[Nle]RCSASVECGQKCLKAIGSIFGKCMNKKCKCYPR-NH₂ |
| 72 | H-S[Nle]D[Nle]RCSASVECVQKCLKAIGSIFGKCMNKKCKCYPR-NH₂ |
| 73 | H-S[Nle]D[Nle]RCSASVECK[2-Amino-5- |
| | carboxypentanoyl]KCLKAIGSIFGKCMNKKCKCYPR-NH₂ |
| 74 | H-S[Nle]D[Nle]RCSASVECKQKCLKAIGSIFGKCMNKKCKCYQ-NH₂ |
| 75 | H-S[Nle]D[Nle]RCSASVECKQKCLKAIGSIFGKCMNKKCRCYPR-NH₂ |
| 76 | H-S[Nle]DERCSASVECKQKCLKAIGSIFGKCMNKKCKCYPR-NH₂ |
| 77 | H-S[Nle]D[Nle]RCSASVECKQKCLGAIGSIFGKCMNKKCKCYPR-NH₂ |
| 78 | H-S[Nle]D[Nle]RCSASVECKQKCLVAIGSIFGKCMNKKCKCYPR-NH₂ |
| 79 | H-S[Nle]D[Nle]RCSASVECAQSCLKAIGSIFGKCMNKKCKCYPR-NH₂ |
| 80 | H-S[Nle]D[Nle]RCSASVECAQKCLAAIGSIFGKCMNKKCKCYPR-NH₂ |
| 81 | H-S[Nle]D[Nle]RCSASVECAQLCLAAIGSIFGKCMNKKCKCYPR-NH₂ |
| 82 | H-S[Nle]D[Nle]RCSASVECKQKCLKAIGSIFGKC[Nle]NKKCKCYPR-OH |
| 83 | H-P[Nle]D[Nle]RCSASVECKQKCLKAIGCIFGKC[Nle]NKKCKCYPC-NH₂ |
| 84 | H-S[Nle]D[Nle]RCSASVECKEKCLQAIGSIFGKC[Nle]NKKCKCYPR-NH₂ |
| 85 | H-P[Nle]D[Nle]RCSASVECKEKCL[homo- |
| | Gln]AIGSIFGKC[Nle]NKKCKCYPR-NH₂ |
| 86 | H-CSASVECKQKCLKAIGCIFGKC[Nle]NKKCKCYPC-NH₂ |
| 87 | H-S[Nle]D[Nle]RCSASVECKQKCLAAIGCIFGKC[Nle]NKKCKCYPC-NH₂ |
| 88 | H-P[Nle]D[Nle]RCSASVECKQKCLKAIGCIFGKC[Nle]NKKCKCYPC-OH |
| 89 | H-S[Nle]D[Nle]RCSALVECKQKCLKAIGSIFGKCMNKKCKCYPR-NH₂ |
| 90 | H-S[Nle]D[Nle]RCSAVVECKQKCLKAIGSIFGKCMNKKCKCYPR-NH₂ |
| 91 | H-S[Nle]D[Nle]RCSASVECKQKCLKAIGSIFGKCMNKKCKC[F(4-F)]PR- |
| | NH₂ |
| 92 | H-S[Nle]D[Nle]RCSASVECKQKCLKAIGSIFGKCMNKKCKC[F(4- |
| | NO₂)]PR-NH₂ |
| 93 | H-S[Nle]D[Nle]RCSASVECKQKCLKAIGSIFGKCMNKKCKC[F(4- |
| | CH₃)]PR-NH₂ |
| 94 | H-[Nle]D[Nle]RCSASVECKQKCLKAIGSIFGKC[Nle]NKKCKCYPR-OH |
| 95 | H-NID[Nle]RCSASVECKQKCLKAIGSIFGKC[Nle]NKKCKCYPR-OH |
| 96 | H-PIE[Nle]RCSASVECKQKCLKAIGSIFGKC[Nle]NKKCKCYPR-OH |
| 97 | H-P[Nle]E[Nle]RCSASVECKQKCLAAIGSIFGKC[Nle]NKKCKCYPR-OH |
| 98 | H-P[Nle]E[Nle]RCSASVECKQKCLAAIGSIFGKC[Nle]NKKCKCYPR-NH₂ |
| 99 | H-P[Nle]E[Nle]RCSASVECKQKCLLAIGSIFGKC[Nle]NKKCKCYPR-OH |
| 100 | H-PIDERCSASVECKQKCLAAIGSIFGKC[Nle]NKKCKCYPR-OH |
| 101 | H-PIE[Nle]RCSASVECKQKCLAAIGSIFGKC[Nle]NKKCKCYPR-OH |
| 102 | H-P[Nle]D[Nle]RCSASVECAQKCLAAIGSIFGKCMNKKCKCYPR-OH |
| 103 | H-P[Nle]E[Nle]RCSASVECAQKCLAAIGSIFGKC[Nle]NKKCKCYPR-OH |
| 104 | H-CSASVECKQKCLKAIGSIFGKC[Nle]NKKCKCYPR-NH₂ |
| 105 | Ac-CSASVECKQKCLKAIGSIFGKC[Nle]NKKCKCYPR-NH₂ |
| 106 | H-RCSASVECKQKCLKAIGSIFGKC[Nle]NKKCKCYPR-NH₂ |
| 107 | Ac-SKCSASVECKQKCLKAIGSIFGKC[Nle]NKKCKCYPR-NH₂ |
| 108 | Ac-LRCSASVECKQKCLKAIGSIFGKC[Nle]NKKCKCYPR-NH₂ |
| 109 | Ac-CSASVECKQKCLKAIGSIFGKC[Nle]NKKCKCYPS-NH₂ |
| 110 | Ac-CSASVECKQKCLKAIGSIFGKC[Nle]NKKCKCYS-NH₂ |
| 111 | Ac-CSASVECKQKCLKAIGSIFGKC[Nle]NKKCKCYG-NH₂ |
| 112 | Ac-CSASVECKQKCLKAIGSIFGKC[Nle]NKKCKCY-NH₂ |
| 113 | Ac-RCSASVECKQKCLKAIGSIFGKC[Nle]NKKCKCYPR-OH |
| 114 | H-LRCSASVECKQKCLKAIGSIFGKC[Nle]NKKCKCYPR-OH |
| 115 | Ac-LRCSASVECKQKCLAAIGSIFGKC[Nle]NKKCKCYPR-OH |
| 116 | Ac-LRCSASVECKQKCLAAIGSIFGKCMNKKCKCYPR-OH |
| 117 | H-CSASVECKQKCLKAIGSIFGKCMNKKCKCYPR-OH |
| 118 | H-P[Nle]E[Nle]RCSASVECKEKCLAAIGSIFGKC[Nle]NKKCKCYPR-OH |
| 119 | H-p[Nle]E[Nle]RCSASVECKQKCLAAIGSIFGKC[Nle]NKKCKCYPR-OH |
| 120 | H-P[Nle]E[Nle]RCSASVECKQKCLAAIGSIFGKC[Nle]NKKCKCYR-OH |
| 121 | H-[Nle]E[Nle]RCSASVECKQKCLAAIGSIFGKC[Nle]NKKCKCYPR-OH |
| 122 | H-P[Nle]E[Nle]RCSASVECKQKCLAAIGSIFGKC[Nle]NKKCKCYPRR-OH |
| 123 | H-P[Nle]E[Nle]RCSASVECKQKCLAAIGSIFGKC[Nle]NKKCKCYPRY-OH |
| 124 | H-P[Nle]E[Nle]RCSASVECKQKCLAAIGSIFGKC[Nle]NKKCKCYPRL-OH |
| 125 | H-P[Nle]E[Nle]RCSASVECKQKCLAAIGSIFGKC[Nle]NKKCKCYPRH-OH |
| 126 | H-P[Nle]E[Nle]RCSASVECKQKCLAAIGSIFGKC[Nle]NKKCKCYPRE-OH |
| 127 | H-P[Nle]E[Nle]RCSASVECKQKCLAAIGSIFGKC[Nle]NKKCKCYPRKS- |
| | OH |
| 128 | H-P[Nle]E[Nle]RCSASVECKQKCLAAIGSIFGKC[Nle]NKKCKCYPRFE- |
| | OH |
| 129 | H-P[Nle]E[Nle]RCSASVECKQKCLAAIGSIFGKC[Nle]NKKCKCYPRHR- |
| | OH |
| 130 | H-P[Nle]E[Nle]RCSASVECKQKCLAAIGSIFGKC[Nle]NKKCKCYPRAK- |
| | OH |
| 131 | H-P[Nle]E[Nle]RCSASVECKQKCLAAIGSIFGKC[Nle]NKKCKCYP-[(4- |
| | amino-5-hydroxypentyl)guanidine] |
| 132 | H-P[Nle]E[Nle]RCSASVECKQKCLAAIGSIFGKC[Nle]NKKCKCYPR-[4- |
| | amino-5-hydroxypentanamide] |
| 133 | H-P[Nle]E[Nle]RCSASVECKQKCLAAIGSIFGKC[Nle]NKKCKCYPRST- |
| | OH |
| 134 | H-P[Nle]E[Nle]RCSASVECKQKCLAAIGSIFGKC[Nle]NKKCKCYPRRY- |
| | OH |
| 135 | H-P[Nle]E[Nle]RCSSSVECKQKCLAAIGSIFGKC[Nle]NKKCKCYPR-OH |
| 136 | H-P[Nle]E[Nle]RCSLSVECKQKCLAAIGSIFGKC[Nle]NKKCKCYPR-OH |
| 137 | H-P[Nle]E[Nle]RCSAPVECKQKCLAAIGSIFGKC[Nle]NKKCKCYPR-OH |
| 138 | H-P[Nle]E[Nle]RCSASPECKQKCLAAIGSIFGKC[Nle]NKKCKCYPR-OH |
| 139 | H-P[Nle]E[Nle]RCSASQECKQKCLAAIGSIFGKC[Nle]NKKCKCYPR-OH |
| 140 | H-P[Nle]E[Nle]RCSASVECLQKCLAAIGSIFGKC[Nle]NKKCKCYPR-OH |
| 141 | H-P[Nle]E[Nle]RCSASVECKQPCLAAIGSIFGKC[Nle]NKKCKCYPR-OH |
| 142 | H-P[Nle]E[Nle]RCEASVECKQKCLAAIGSIFGKC[Nle]NKKCKCYPR-OH |
| 143 | H-P[Nle]E[Nle]RCFASVECKQKCLAAIGSIFGKC[Nle]NKKCKCYPR-OH |
| 144 | H-P[Nle]E[Nle]RCSYSVECKQKCLAAIGSIFGKC[Nle]NKKCKCYPR-OH |
| 145 | H-P[Nle]E[Nle]RCSAFVECKQKCLAAIGSIFGKC[Nle]NKKCKCYPR-OH |

The following compounds were also synthesised for use as controls:

| | |
|---|---|
| ShK | H-RSCIDTIPKSRCTAFQCKHSMKYRLSFCRKTCGTC-OH |
| ShK186 | H-[phosphotyrosyl)][8-Amino-3,6- |
| | dioxaoctanoyl]RSCIDTIPKSRCTAFQCKHSMKYRLS |
| | FCRKTCGTC-NH₂ |
| Moka1 | H-INVKCSLPQQCIKPCKDAGMRFGKCMNKKCRCYS-OH |
| Vm24 | H-AAAISCVGSPECPPKCRAQGCKNGKCMNRKCKCYYC-NH₂ |

### Example 2: Kv1.3 Blocker Activity in FLIPR Thallium Assay

A human Kv1.3 voltage-gated K+ channel cell line was purchased from Perkin Elmer (TDS-AX-010-C-1). The cell line is based on CHO-DUKX cells stably transfected with the human Kv1.3 voltage-gated K+ channel.

The cell line was grown in MEMα with nucleotides, GlutaMAX (Gibco Cat #32571028), 10% Foetal Bovine Serum (FBS), 0.4 mg/ml Geneticin, 100 units/ml Penicillin, and 100 μg/ml Streptomycin and seeded at 10.000 cells/well in black poly-D-lysine-coated 96 well plates.

The FluxOR™ Potassium Ion Channel Assay (Invitrogen Cat #F10016) was used to quantitate flux of thallium ions into the cells as a response to Kv1.3 activation with a stimulus buffer causing depolarization of the cell membrane, generating a fluorescent signal, proportional to channel activity. The assay was performed as described by the assay kit manufacturer. Fluorescence responses were recorded and quantified using the FLIPR® Tetra High Throughput Screening System (Molecular Devices, Inc.).

Data from test compounds eliciting an inhibition of thallium flux into the cell were normalised relative to the positive (ShK) and negative control (vehicle) to calculate the IC₅₀ from the concentration response curve. Results are shown in Table 2. IC₅₀ can be regarded as a measure of potency of inhibition for the respective compound. The IC₅₀ value is a measure of the concentration of an inhibitor required to achieve half of that compound's maximal inhibition of ion channel activity in a given assay. A compound which has a lower IC₅₀ at a particular ion channel than a reference compound can be considered to be a more active inhibitor, or a more potent inhibitor, than the reference compound.

TABLE 2

| Compound | IC50 (nM) |
|---|---|
| 1 | 0.24 |
| 2 | 0.25 |
| 3 | 0.30 |
| 4 | 0.29 |
| 5 | 0.25 |
| 6 | 0.34 |
| 7 | 0.12 |
| 8 | 0.47 |
| 9 | 0.53 |
| 10 | 0.57 |
| 11 | 0.3 |
| 12 | 0.23 |
| 13 | 0.26 |
| 14 | 0.16 |
| 15 | 0.12 |
| 16 | 0.14 |
| 17 | 0.14 |
| 18 | 0.19 |
| 19 | 0.12 |
| 20 | 0.13 |
| 21 | 0.12 |
| 22 | 0.11 |
| 23 | 0.055 |
| 24 | 0.073 |
| 25 | 0.085 |
| 26 | 0.092 |
| 27 | 0.065 |
| 28 | 0.064 |
| 29 | 0.18 |
| 30 | 0.09 |
| 31 | 0.075 |
| 32 | 0.12 |
| 33 | 0.14 |
| 34 | 0.11 |
| 35 | 0.077 |
| 36 | 0.24 |
| 37 | 0.14 |
| 38 | 0.46 |
| 39 | 0.11 |
| 40 | 0.1 |
| 41 | 0.15 |
| 42 | 0.087 |
| 43 | 0.5 |
| 44 | 0.13 |
| 45 | 0.16 |
| 46 | 0.11 |
| 47 | 0.12 |
| 48 | 0.14 |
| 49 | 0.36 |
| 50 | 0.16 |
| 51 | 0.11 |
| 52 | 0.11 |
| 53 | 0.12 |
| 54 | 0.46 |
| 55 | 0.12 |
| 56 | 0.087 |
| 57 | 0.11 |
| 58 | 0.11 |
| 59 | 0.035 |
| 60 | 0.035 |
| 61 | 0.40 |
| 62 | 0.13 |
| 63 | 0.13 |
| 64 | 0.12 |
| 65 | 0.12 |
| 66 | 0.12 |
| 67 | 0.12 |
| 68 | 0.20 |
| 69 | 0.58 |
| 70 | 0.13 |
| 71 | 0.10 |
| 72 | 0.19 |
| 73 | 0.11 |
| 74 | 0.36 |
| 75 | 0.10 |
| 76 | 0.093 |
| 77 | 0.13 |
| 78 | 0.12 |
| 79 | 0.13 |
| 80 | 0.15 |
| 81 | 0.22 |
| 82 | 0.49 |
| 83 | 0.12 |
| 84 | 0.084 |
| 85 | 0.15 |
| 86 | 0.20 |
| 87 | 0.10 |
| 88 | 0.27 |
| 89 | 2.7 |
| 90 | 0.19 |
| 91 | 0.10 |
| 92 | 0.49 |
| 93 | 0.13 |
| 94 | 0.16 |
| 95 | 0.16 |
| 96 | 0.15 |
| 97 | 0.15 |
| 98 | 0.11 |
| 99 | 0.34 |
| 100 | 0.85 |
| 101 | 0.18 |
| 102 | 0.45 |
| 103 | 0.38 |
| 104 | 0.12 |
| 105 | 0.16 |
| 106 | 0.092 |
| 107 | 0.084 |
| 108 | 0.1 |
| 109 | 0.96 |
| 110 | 2.3 |
| 111 | 2.1 |
| 112 | 1.7 |
| 113 | 0.34 |
| 114 | 0.39 |
| 115 | 0.31 |
| 116 | 0.18 |
| 117 | 1.5 |
| 118 | 1.39 |
| 119 | 0.63 |
| 120 | 0.894 |
| 121 | 0.451 |
| 122 | 0.226 |
| 123 | 1.04 |
| 124 | 1.47 |
| 125 | 0.383 |
| 126 | 6.49 |
| 127 | 0.562 |
| 128 | 7.18 |
| 129 | 0.143 |
| 130 | 0.529 |
| 131 | 0.156 |
| 132 | 0.224 |
| 133 | 1.1 |
| 134 | 0.21 |
| 135 | 0.336 |
| 136 | 6.24 |
| 137 | 0.333 |
| 138 | 0.228 |
| 139 | 0.453 |
| 140 | 3.57 |
| 141 | 1.07 |
| 142 | 1.5 |
| 143 | 0.316 |
| 144 | 0.739 |
| 145 | 8.26 |

### Example 3: Kv1.3 Selectivity in Patch Clamp Assay

Chinese Hamster Ovary (CHO) cell lines stably expressing exogenous human α-subunits of each potassium ion channel were grown and passaged under standard culture conditions.

The automated, chip-based planar patch clamp device QPatch® was used to quantitate the ionic currents. All recordings were made in the conventional whole-cell configuration after establishment of gigaohm seals. External recording solution contained (150 mM NaCl, 10 mM KCl, 10 mM HEPES, 1 mM MgCl₂, 3 mM CaCl₂, 10 mM Glucose, pH adjusted to 7.4 with NaOH) and Internal recording solution (20 mM KCl, 120 mM KF, 10 mM HEPES, 10 mM EGTA, 5 mM NaATP, pH adjusted to 7.2 with KOH). During experiments 0.1% (v/v) BSA was included as a vehicle in all external recording solutions. Currents were elicited from a holding potential of −80 mV using a voltage protocol, which shifted the voltage to 30 mV for 500 ms every 15 s. Concentration-response relationships were established by cumulatively applying seven escalating concentrations of test sample to an individual cell with a recording period of 2 min per compound application.

The efficacy was determined as the mean charge for the last three sweeps at the end of each concentration application period from the cursor positions. The percent inhibition for each test dose application period was calculated as the reduction in mean cursor value (charge) relative to the cursor value measured at the end of the vehicle period and used to calculate the IC₅₀ from the concentration response curve.

Results are shown in Table 3.

TABLE 3

| | Potency IC₅₀ (nM) | | | | Selectivity | | |
|---|---|---|---|---|---|---|---|
| Compound | Kv1.1 | Kv1.2 | Kv1.3 | Kv1.6 | Kv1.1/Kv1.3 | Kv1.2/Kv1.3 | Kv1.6/Kv1.3 |
| 1 | >3000 | 518 | 0.38 | >3000 | >7900 | 1364 | >7900 |
| 2 | >3000 | 97 | 1.50 | 1810 | >2000 | 65 | 1204 |
| 3 | >3000 | 409 | 0.27 | >3000 | >11300 | 1540 | >11300 |
| 5 | >3000 | 285 | 0.38 | >3000 | >7900 | 747 | >7900 |
| 8 | >3000 | 376 | 0.44 | >3000 | >6800 | 857 | >6800 |
| 12 | >3000 | 228 | 0.11 | >3000 | >27600 | 2093 | >27600 |
| 16 | >3000 | 133 | 0.24 | 2460 | >12600 | 561 | 10360 |
| 17 | >3000 | 66 | 0.42 | 912 | >7200 | 159 | 2182 |
| 18 | >3000 | 256 | 0.24 | >3000 | >12700 | 1080 | >12700 |
| 19 | >3000 | 45 | 0.22 | 435 | >13500 | 202 | 1955 |
| 20 | >3000 | 99 | 0.21 | 2527 | >14300 | 471 | 12045 |
| 21 | >3000 | 57 | 0.22 | 626 | >13700 | 259 | 2851 |
| 22 | >3000 | 181 | 0.29 | >3000 | >10200 | 615 | >10200 |
| 23 | 626 | 28 | 0.15 | 215 | 4280 | 191 | 1472 |
| 25 | >3000 | 67 | 0.09 | 363 | >34200 | 761 | 4144 |
| 26 | >3000 | 43 | 0.26 | >3000 | >11800 | 169 | >11800 |
| 27 | >3000 | 11 | 0.12 | 206 | >25900 | 94 | 1778 |
| 28 | >3000 | 16 | 0.20 | 252 | >15200 | 83 | 1272 |
| 29 | >3000 | 874 | 0.40 | >3000 | >7400 | 2163 | >7400 |
| 30 | >3000 | 666 | 0.14 | >3000 | >22000 | 4897 | >22000 |
| 31 | >3000 | 179 | 0.28 | >3000 | >10800 | 648 | >10800 |
| 32 | | | 0.38 | | | | |
| 34 | >3000 | 100 | 0.42 | 207 | >7100 | 237 | 490 |
| 35 | >3000 | 18 | 0.20 | 192 | >15000 | 88 | 963 |
| 36 | >3000 | 439 | 0.31 | >3000 | >9700 | 1422 | >9700 |
| 37 | >3000 | 338 | 0.19 | >3000 | >16100 | 1816 | >16100 |
| 39 | | | 0.15 | | | | |
| 40 | >3000 | 113 | 0.26 | 1829 | >11600 | 436 | 7073 |
| 41 | >3000 | 939 | 0.18 | >3000 | >17100 | 5362 | >17100 |
| 42 | >3000 | 109 | 0.16 | 316 | >18600 | 674 | 1954 |
| 45 | >3000 | 257 | 0.75 | >3000 | >4000 | 343 | >4000 |
| 46 | >3000 | 302 | 0.32 | 1395 | >9400 | 943 | 4359 |
| 47 | >3000 | 772 | 0.54 | 609 | >5600 | 1438 | 1134 |
| 48 | >3000 | >3000 | 0.37 | >3000 | >8200 | >8200 | >8200 |
| 49 | >3000 | 164 | 1.34 | 946 | >2200 | 123 | 707 |
| 50 | | | 0.27 | | | | |
| 51 | | | 0.25 | | | | |
| 52 | >3000 | 88 | 0.11 | >3000 | >27500 | 803 | >27500 |
| 53 | >3000 | 149 | 0.29 | >3000 | >10300 | 514 | >10300 |
| 54 | >3000 | 291 | 2.41 | >3000 | >1200 | 121 | >1200 |
| 55 | | | 0.12 | | | | |
| 56 | >3000 | 57 | 0.30 | 932 | >9900 | 187 | 3062 |
| 58 | >3000 | 145 | 0.34 | >3000 | >8800 | 426 | >8800 |
| 62 | >3000 | 449 | 0.26 | 2349 | >11600 | 1740 | 9102 |
| 63 | >3000 | 184 | 0.31 | 1231 | >9600 | 587 | 3929 |
| 64 | | | 0.25 | | | | |
| 65 | | | 0.14 | | | | |
| 66 | | | 0.18 | | | | |
| 67 | | | 0.32 | | | | |
| 70 | >3000 | 423 | 0.25 | >3000 | >12200 | 1720 | >12200 |
| 71 | >3000 | 373 | 0.11 | >3000 | >27700 | 3438 | >27700 |
| 75 | | | 0.13 | | | | |
| 76 | >3000 | 122 | 0.28 | >3000 | >10600 | 430 | >10600 |
| 79 | >3000 | 1149 | 0.21 | >3000 | >14600 | 5596 | >14600 |
| 80 | | | 0.16 | | | | |
| 83 | >3000 | 624 | 0.63 | 1222 | >4800 | 995 | 1949 |
| 94 | >3000 | 1859 | 0.17 | >3000 | >17800 | 11034 | >17800 |
| 95 | >3000 | 2063 | 0.27 | >3000 | >11300 | 7756 | >11300 |
| 96 | >3000 | 1758 | 0.39 | >3000 | >7800 | 4561 | >7800 |
| 97 | >3000 | 1117 | 0.33 | >3000 | >9100 | 3384 | >9100 |
| 98 | >3000 | 311 | 0.18 | >3000 | >16200 | 1689 | >16200 |
| 101 | >3000 | 1968 | 0.35 | >3000 | >8600 | 5630 | >8600 |
| 103 | >3000 | >3000 | 0.55 | >3000 | >5500 | 5500 | >5500 |
| 104 | >3000 | >3000 | 0.43 | >3000 | >7000 | >7000 | >7000 |
| 105 | >3000 | 384 | 0.24 | >3000 | >12400 | 1591 | >12400 |
| 106 | >3000 | 280 | 0.40 | 1302 | >7600 | 708 | 3290 |
| 107 | >3000 | 196 | 1.00 | 1451 | >3000 | 196 | 1451 |
| 108 | >3000 | 122 | 0.35 | 2456 | >8700 | 352 | 7096 |
| 117 | >3000 | 1832 | 2.09 | >3000 | >1400 | 878 | >1400 |
| ShK | 0.0021 | 11 | 0.017 | 0.19 | 0.12 | 644 | 11 |
| ShK-186 | 0.31 | 43 | 0.095 | 0.45 | 4.9 | 682 | 7.4 |
| Moka1 | >3000 | 275 | 9.0 | >3000 | >300 | 31 | >300 |
| Vm24 | 0.097 | 4.7 | 0.15 | 21 | 0.65 | 31 | 139 |

Comparison of the data in Examples 2 and 3 shows that a good correlation exists between the IC₅₀ values as measured in the two assays.

### Example 4a: Inhibitory Activity of Kv1.3 Blockers on Human PBMCs

Human peripheral blood mononuclear cells (PBMCs) were used to assess the effects of Kv1.3 blockers on T-cell activation as determined by IL-2 (cytokine) release after stimulation with anti-CD3.

Human PBMCs were obtained from Precision for Medicine (Frederick, MD). Cells from 5 donors were used. Plate-bound anti-CD3 was used to stimulate bulk T cells in the PBMC preparations. Briefly, 96-well plates were coated with anti-CD3 antibody for 2 hrs at 37° C., using 50 μL of a 0.5 μg/mL anti-CD3 solution diluted in 1×PBS. Thereafter the plates were washed twice.

Kv1.3 blockers as shown in Table 4a were diluted in medium (RPMI 1640 with Glutamax-I containing 10% v/v Fetal Bovine Serum, 1% v/v penicillin-streptomycin solution) and added in a volume of 100 μL at concentrations ranging from 0.01 μM to 100 nM (tenfold dilutions). Cyclosporin A (1 ug/ml) and Vm24 peptide (100 nM) were used as positive controls. Finally, 1×10⁵ PBMCs were added to each well in a volume of 100 μL, giving a final volume of 200 μL per well. The plates were incubated for 20-24 hours in a 37° C./5% CO₂ incubator. After centrifugation of the plates, 25 μl supernatant was transferred to IL-2 detection plates (MSD Human IL-2 Tissue Culture Kit, cat #K151AHB-2) and IL-2 was measures as described by the manufacturer (Meso Scale Discovery, Rockville, Maryland, USA).

Results are shown in Table 4a as geometric mean of IC50 values obtained from anti-CD3 stimulated human PBMC assays. All values derive from at least 4 replicates.

TABLE 4a

| | anti-CD3 PBMC/IL-2 |
|---|---|
| Compound | IC50 [nM] |
| ShK-186 | 0.07 |
| 16 | 0.05 |
| 17 | 0.4 |
| 20 | 0.1 |
| 35 | 0.1 |

Incubation with anti-CD3 antibody activated hPBMC and addition of Kv1.3 blockers resulted in dose-dependent reduction in the IL-2 secretion. On average the IC₅₀ values (as calculated from IL-2 release) of the test compounds were in the range of 0.05 nM to 0.4 nM. This was comparable to the IC₅₀ observed with ShK186 (IC₅₀ is 0.07 nM) and about 10-100 fold lower than the IC₅₀ of Moka1 which was less potent in inhibiting IL-2 secretion.

There is no significant difference between the test compounds and ShK186. ShK186 and test compounds were all significantly lower than Moka1.

Cyclosporine blocked CD3 induced IL-2 release completely in all experiments.

### Example 4b: Inhibitory Activity of Kv1.3 Blockers on Human PBMCs

Human peripheral blood mononuclear cells (PBMCs) were used to assess the effects of Kv1.3 blockers on T-cell activation as determined by IL-2 release after stimulation with anti-CD3.

Human PBMCs were obtained from Precision for Medicine (Frederick, MD). Cells from 5 donors were used. Plate-bound anti-CD3 was used to stimulate bulk T cells in the PBMC preparations. Briefly, 96-well plates were coated with anti-CD3 antibody for app. 16 hours at 5° C., using 50 μl of a 1 μg/ml anti-CD3 solution diluted in PBS. Thereafter the plates were washed twice.

Kv1.3 blockers were subsequently diluted in medium (RPMI 1640 with Glutamax-I containing, 10% v/v Fetal Bovine Serum, 1% v/v penicillin-streptomycin Solution) and added in a volume of 50 μl. The compounds indicated in Table 4b were used at concentrations ranging from 0.3 μM to 1000 nM (half log dilutions, starting concentrations varying). Cyclosporin A (1 μg/ml) and Vm24 peptide (100 nM) were used as positive controls.

Finally, 50.000 PBMCs in the same medium were added to each well in a volume of 50 μl, giving a final volume of 100 μl per well. The plates were incubated for 20-24 hours in a 37° C./5% CO₂ incubator. After centrifugation of the plates, 25 μl supernatant was transferred to IL-2 detection plates (MSD Human IL-2 Tissue Culture Kit, cat #K151AHB-2) and IL-2 was measures as described by the manufacturer (Meso Scale Discovery, Rockville, Maryland, USA).

Results are shown in Table 4b as geometric mean of IC50 values obtained from anti-CD3 stimulated human PBMC assays. All values derive from at least 6 replicates.

TABLE 4b

| | anti-CD3 PBMC/IL-2 |
|---|---|
| Compound | IC50 [nM] |
| ShK-186 | 0.04 |
| 3 | 0.06 |
| 1 | 0.09 |
| 41 | 0.03 |
| 23 | 0.06 |
| 48 | 0.07 |

Incubation with anti-CD3 antibody activated hPBMC and addition of the Compounds of this invention resulted in dose-dependent reduction in the IL-2 secretion. The average IC₅₀ values (as calculated from IL-2 release) of the test compounds were in the range of 0.01 nM to 0.09 nM as shown in Table 4b. This was comparable to the IC₅₀ observed with ShK186 (IC₅₀ is 0.05 nM). This assay was performed using different donors than those used for Example 4a, so identical values for the Shk-186 in the two sets of experiments are not expected.

Cyclosporine blocked anti-CD3 induced IL-2 release completely in all experiments.

### Example 5: Inhibitory Activity of Kv1.3 Blockers in Rat Whole Blood

Rat whole blood was used to assess the potency of Kv1.3 blockers on T-cell activation as determined by IL-17A release after stimulation with thapsigargin. Addition of thapsigargin results in activation of a signalling cascade ending up in activation of T cell proliferation and cytokine production where the Kv1.3 ion channel plays a key role, so activity of Kv1.3 blockers in primary cells can be measured in this experimental system.

Rat whole blood was obtained from healthy, naïve Lewis or Sprague-Dawley rats that were terminally bleed from the heart using Sodium Heparin blood sampling tubes for collection. Test compounds were diluted to 4× final testing concentrations in assay buffer (DMEM+GlutaMAX), GlutaMAX is a medium comprising 3.97 mM L-alanine-L-glutamine (Gibco Cat #61965026) supplemented with 25 mM HEPES buffer, 1 mM Sodium Pyruvate, 100 units/ml Penicillin, 100 μg/ml Streptomycin and 0.05% Casein from bovine milk (Sigma-Aldrich)) and 25 μl was added to wells of a 96 well plate. Then 50 μl whole rat blood was added and incubated for minimum 5 minutes at room temperature to allow compound binding. Then 25 μl 40 μM thapsigargin diluted in assay buffer was added to all wells of the assay plates to activate the cells, followed by incubation for 24 Hr at 37° C./5% CO₂ in a humidified box. The assay plates were centrifuged for 10 min at 300 g at 4° C. and the supernatants were transferred to new plates. The concentrations of IL-17A released to the supernatants were measured using a Rat IL-17A ELISA Kit (Abcam Cat #ab214028) as recommended by the manufacturer. Samples were diluted 2.5-fold by transferring 20 μl of the supernatants to wells on ELISA plates containing 30 μl buffer 75BS from the detection kit. Data from test compounds eliciting an inhibition of IL-17A were normalised relative to full thapsigargin activation (no blocker added) and no activation controls (addition of assay buffer instead of thapsigargin) to calculate the IC₅₀ from the concentration response curve.

Results are shown in Table 5, expressed as IC₅₀, with standard deviation (IC₅₀_SD). All values are derived from at least 2 replicates. The biological effects ex vivo show a correlation with the potency of the compounds.

TABLE 5

| CPD NO | IC₅₀ | IC_{50 —}SD |
|---|---|---|
| 1 | 0.17 | 0.036 |
| 3 | 1.0 | 0.31 |
| 17 | 0.88 | 0.32 |
| 18 | 3.2 | 0.017 |
| 19 | 0.56 | 0.2 |
| 20 | 1.2 | 0.72 |
| 21 | 0.82 | 0.81 |
| 22 | 2.8 | 2.5 |
| 23 | 0.57 | 0.24 |
| 26 | 0.61 | 0.33 |
| 27 | 0.68 | 0.47 |
| 28 | 0.53 | 0.21 |
| 29 | 0.76 | 0.23 |
| 30 | 0.41 | 0.021 |
| 31 | 0.86 | 0.31 |
| 32 | 0.75 | 0.24 |
| 33 | 0.70 | 0.40 |
| 34 | 2.1 | 0.89 |
| 35 | 2.5 | 2.1 |
| 36 | 1.2 | 0.7 |
| 37 | 0.77 | 0.56 |
| 38 | 2.8 | 1.8 |
| 41 | 0.98 | 0.60 |
| 42 | 0.91 | 0.26 |
| 43 | 0.99 | 0.24 |
| 44 | 1.1 | 0.47 |
| 45 | 1.4 | 0.64 |
| 46 | 0.79 | 0.28 |
| 47 | 0.83 | 0.4 |
| 48 | 0.63 | 0.039 |
| 49 | 1.0 | 0.65 |
| 52 | 0.69 | 0.21 |
| 62 | 0.44 | 0.080 |
| 63 | 0.68 | 0.19 |
| 64 | 1.2 | 0.71 |
| 65 | 1.1 | 0.011 |
| 66 | 0.47 | 0.35 |
| 67 | 0.98 | 0.076 |
| 68 | 0.93 | 0.72 |
| 69 | 2.7 | 0.54 |
| 70 | 0.79 | 0.15 |
| 71 | 0.30 | 0.11 |
| 72 | 0.47 | 0.086 |
| 73 | 0.52 | 0.019 |
| 74 | 1.6 | 1.1 |
| 75 | 0.86 | 0.13 |
| 76 | 0.55 | 0.25 |
| 78 | 0.39 | 0.28 |
| 82 | 0.52 | 0.059 |
| 83 | 0.81 | 0.13 |
| 84 | 0.69 | 0.38 |
| 85 | 1.4 | 1.1 |
| 86 | 1.1 | 1.3 |
| 87 | 0.73 | 0.28 |
| 88 | 1.1 | 1.2 |
| 91 | 0.34 | 0.07 |
| 92 | 1.3 | 1.0 |
| 93 | 0.54 | 0.31 |
| 94 | 0.55 | 0.42 |
| 95 | 1.2 | 0.57 |
| 96 | 1.8 | 1.2 |
| 97 | 0.43 | 0.42 |
| 98 | 0.34 | 0.21 |
| 99 | 0.5 | 0.37 |
| 100 | 0.87 | 0.84 |
| 101 | 0.61 | 0.34 |
| 102 | 0.32 | 0.22 |
| 103 | 1.2 | 1.5 |
| 104 | 1.6 | 0.94 |
| 105 | 0.88 | 0.011 |
| 106 | 0.52 | 0.34 |
| 107 | 0.36 | 0.13 |
| 108 | 0.37 | 0.049 |
| 114 | 2.1 | 2.5 |
| 115 | 0.9 | 0.3 |
| 116 | 0.36 | 0.24 |
| 117 | 1.9 | 1 |

### Example 6: Pharmacokinetic Characterisation

Method

Sprague Dawley or Wistar rats (males with a body weight of approximately 250-350 g) were given a single subcutaneous (s.c.) injection of each peptide to be tested.

Following s.c. administration of the selected compounds (dose 70 nmol/kg, dosing volume either 2 or 5 mL/kg), blood samples were drawn at 15 min, 30 min, 45 min, 60 min, 90 min, 2 h, 3 h, 4 h post-dose. At each sampling time point, samples from the rats were drawn by sublingual bleeding or by tail cut. After last sampling the rats were sacrificed by O₂/CO₂ anaesthesia. The dosing vehicle was 10 mM phosphate, 0.8% NaCl, 0.05% Polysorbate 20 (pH 6.0).

Plasma samples were analyzed after solid phase extraction (SPE) by liquid chromatography mass spectrometry (LC-MS/MS). Mean plasma concentrations were used for calculation of the pharmacokinetic parameters using the non-compartmental approach in Phoenix WinNonlin 6.4 or a later version. Plasma terminal elimination half-life (T½) was determined as In(2)/λz where λz is the magnitude of the slope of the log linear regression of the log concentration versus time profile during the terminal phase. AUC_{inf} is the area under the plasma concentration—time curve extrapolated to infinity (AUC_{inf}=AUCₗₐₛₜ+Cₗₐₛₜ/λz, where Cₗₐₛₜ is the last observed plasma concentration). Cmax is the maximum observed concentration, occurring at Tmax. Results for selected compounds are shown in table 6.

TABLE 6

| Cpd. | Dose | AUC_{INF} | Cmax | T_{1/2} |
|---|---|---|---|---|
| No. | nmol/kg | (hr*nmol/L) | (nmol/L) | (hr) |
| 3 | 70 | 83.9 | 36.9 | 0.99 |
| 8 | 70 | 98.3 | 76.9 | 0.62 |
| 30 | 70 | 46.7 | 33.3 | 0.74 |
| 52 | 70 | 48.5 | 31.2 | 0.89 |
| 53 | 70 | 31.1 | 50.0 | 0.87 |
| 95 | 70 | 85.0 | 46.5 | 1.09 |
| 97 | 70 | 112.0 | 52.6 | 1.04 |
| 101 | 70 | 131.8 | 58.6 | 0.91 |
| 103 | 70 | 176.6 | 68.4 | 1.36 |
| 5 | 70 | 72.4 | 48.4 | 0.93 |

### Example 7: Effect of Kv1.3 Blocker Treatment in the Keyhole Limpet Hemocyanin (KLH) Ear Inflammation Model in Rats

A classical delayed-type hypersensitivity (DTH) reaction was elicited in one ear of rats. Briefly, male Lewis rats aged 8-10 weeks were immunized on day −7 with 200 μL keyhole limpet hemocyanin (KLH) (from Sigma, cat. no. H7017) (4 mg/mL) emulsified in complete Freund's adjuvant (CFA) (Difco, cat. no. 263810) subcutaneously (SC) at the base of the tail. On day 0 the rats were challenged intradermally with 40 μL KLH/NaCl 0.9% (2 mg/mL) in the left ear. After the ear challenge the rats develop a T-cell dependent inflammation in the left KLH challenged ear. The right ear remains uninflamed and serves as control.

The ability of Kv1.3 blocker treatment to reduce the DTH ear swelling response was investigated by comparing the response in rats (n=8-10/gr) treated with vehicle to that of rats treated with a Kv1.3 blocker. Vehicle or Kv1.3 blocker dissolved in vehicle was administered SC (2 mL/kg) 24 hrs prior to KLH ear challenge. The test dose of Kv1.3 blocker was 50, 70 or 100 nmol/kg. Test vehicle was 10 mM phosphate, 0.8% w/v NaCl, 0.05% w/v polysorbate20, pH 6. Cyclosporine (CsA) was included as positive study control in all experiments. Cyclosporine (Sandimmune Neooral® 100 mg/mL oral solution, Novartis) was administered per os (10 mg/kg) one hour prior to KLH ear challenge and again 6 hours after KLH ear challenge.

As primary read-out of efficacy, the Area Under Curve (AUC) of A ear thickness (mm) was calculated for each animal from 0-48 hours post induction of the ear DTH reaction, where the change (D) was calculated as: Left ear thickness−right ear thickness. These results were then used to calculate % inhibition of ear thickness by Kv1.3 blocker treatment: % inhibition: ((1−(individual Δ AUC Kv1.3 blocker/average ΔAUC vehicle group))×100. Results were calculated as % inhibition+/−standard deviation (SD), and are shown in Table 7 and Table 8.

TABLE 7

| | Dose | | | | |
|---|---|---|---|---|---|
| Exp | (nmol/kg) | Cpd. 3 | Cpd. 41 | Cpd. 52 | CsA* |
| #1 | 70 | 38.8 | 46.9 | | 71.4 |
| | | (+/−9.7) | (+/−10.9) | | (+/−4.8) |
| #2 | 70 | 25.0 | 27.7 | | 76.8 |
| | | (+/−12.5) | (+/−11.5) | | (+/−13.4) |
| #3 | 50 | 37.3 | 22.2 | | 65.1 |
| | | (+/−13.8) | (+/−11.1) | | (+/−4.5) |
| #4 | 100 | | 41.9 | 25.1 | 63.4 |
| | | | (+/−12.5) | (+/−6.3) | (+/−5.9) |

TABLE 8

| | Dose | | | | | |
|---|---|---|---|---|---|---|
| Exp. | (nmol/kg) | Cpd. 95 | Cpd. 97 | Cpd. 101 | Cpd. 103 | CsA |
| #5 | 100 | 39.6 | | | | 67.1 |
| | | (+/−8.3) | | | | (+/−7.9) |
| #6 | 100 | | 42.5 | 44.5 | | 55.3 |
| | | | (+/−14.0) | (+/−5.2) | | (+/−5.5) |
| #7 | 100 | | | | 48.9 | 67.9 |
| | | | | | (+/−8.6) | (+/−5.4) |
| #8 | 100 | | 37.7 | | | 57.8 |
| | | | (+/−10.0) | | | (+/−2.8) |

The work described in this specification has received funding from the European Communities Seventh Framework Program FP7/2007-2013 under grant agreement Venomics_CA_20111021.

1. An ion-channel blocker comprising a Kv1.3 inhibitor component, said Kv1.3 inhibitor component comprising:
a variant of the sequence of PaT1:
(SEQ ID NO. 1)
QMDMRCSASVECKQKCLKAIGSIFGKCMNKKCKCYPR;
which variant differs from PaT1 by from 1 to 9 substitutions, insertions or deletions, wherein any substitutions or deletions are at amino acid positions selected from positions 1-5, 7-11, 13-15, 17-23, 25, 28-31, 33 and 35-37;
wherein if the residue at position 23 is different from that of PaT1 then the residue at position 23 is selected from R, K, hK, P and H; and
wherein said Kv1.3 inhibitor component has Kv1.3 inhibitor activity and is selective for Kv1.3;
or a pharmaceutically acceptable salt thereof.

2. An ion channel blocker or pharmaceutically acceptable salt according to claim 1 wherein one or more of positions 28, 31, 34, 35, 36 or 37, or one or more of positions 28, 31, 34 and 35, are identical to the corresponding position in PaT1.

3. An ion channel blocker or pharmaceutically acceptable salt according to claim 2 wherein all of positions 28, 31, 34 and 35, or all of positions 28, 31, 34, 35, 36 and 37, are identical to the corresponding positions in PaT1.

4. An ion channel blocker or pharmaceutically acceptable salt according to claim 1, wherein any substitutions or deletions are made only at the following positions wherein, if different from PaT1:
the residue at position 1 is selected from N, P, I, V, H, Y and S or is deleted;
the residue at position 2 is selected from Nle, I, V, L, T, Q and E or is deleted;
the residue at position 3 is selected from E, S and N or is deleted;
the residue at position 4 is selected from Nle, I, V, L, A, E and S or is deleted;
the residue at position 5 is selected from K, Q, A, L, E and D or is deleted;
the residue at position 7 is selected from K, R, Q, E, F and A;
the residue at position 8 is selected from I, H, S, L, Y and G;
the residue at position 9 is selected from A, Abu, P, F, V and L;
the residue at position 10 is selected from R, K, P, A, Q and L;
the residue at position 11 is selected from Q and D;
the residue at position 13 is selected from L, A, E, Q, I, H, D, V and G;
the residue at position 14 is selected from V, K, E, L, D and 2-amino-5-carboxypentanoyl;
the residue at position 15 is selected from S, L and P;
the residue at position 17 is selected from K, Y, R, Q, D, V and E or is deleted;
the residue at position 18 is selected from D, A, Y, G, V, Q, hQ and L or is deleted;
the residue at position 19 is selected from R, Y and Q or is deleted;
the residue at position 20 is selected from Y, E and R or is deleted;
the residue at position 21 is selected from R, H, E and D;
the residue at position 22 is selected from R, M, D, L and C;
the residue at position 23 is selected from R, K, hK, P and H;
the residue at position 26 is homo-lysine;
the residue at position 28 is selected from Nle, A and L;
the residue at position 29 is V;
the residue at position 30 is selected from G and D;
the residue at position 31 is selected from D, Q, E and H;
the residue at position 33 is selected from H, V, D, Q and R;
the residue at position 35 is selected from T, F(4-NH₂), F(4-F), F(4-NO₂) and F(4-CH₃);
the residue at position 36 is selected from Q, S and G or is deleted;
the residue at position 37 is selected from K, E, S and C or is deleted.

5. An ion channel blocker or pharmaceutically acceptable salt according to claim 4, wherein any substitutions or deletions are made only at the following positions wherein, if different from PaT1:
the residue at position 1 is selected from N, P, I, V, H, Y and S or is deleted;
the residue at position 2 is selected from Nle, I, V, L, T, Q and E or is deleted;
the residue at position 3 is selected from E, S and N or is deleted;
the residue at position 4 is selected from Nle, I, V, L, A, E and S or is deleted;
the residue at position 5 is selected from K, Q, A and L or is deleted;
the residue at position 7 is selected from K, R and Q;
the residue at position 8 is selected from I, H, S, L, Y and G;
the residue at position 9 is selected from A, Abu, P, F, V and L;
the residue at position 10 is selected from R, K and P;
the residue at position 11 is Q;
the residue at position 13 is selected from L, A, E, Q, I, H and G;
the residue at position 14 is selected from V, K, E, L and 2-amino-5-carboxypentanoyl;
the residue at position 15 is selected from S and L;
the residue at position 17 is selected from K, Y, R, Q and D or is deleted;
the residue at position 18 is selected from D, A, Y, G, V, Q, hQ and L or is deleted;
the residue at position 19 is selected from R, Y and Q or is deleted;
the residue at position 20 is selected from Y, E and R or is deleted;
the residue at position 21 is selected from R, H and E;
the residue at position 22 is selected from R, M, D, L and C;
the residue at position 23 is selected from R, K, hK and P;
the residue at position 28 is Nle;
the residue at position 30 is selected from G and D;
the residue at position 31 is selected from D, Q, E and H;
the residue at position 33 is selected from H, V, D, Q and R;
the residue at position 35 is selected from T, F(4-NH₂), F(4-F), F(4-NO₂) and F(4-CH₃);
the residue at position 36 is selected from Q, S and G or is deleted;
the residue at position 37 is selected from K, S and C or is deleted.

6. An ion channel blocker or pharmaceutically acceptable salt according to claim 5, wherein any substitutions or deletions are made only at the following positions wherein, if different from PaT1:
the residue at position 1 is selected from N, P, V, H, Y and S or is deleted;
the residue at position 2 is selected from Nle, I, V and L or is deleted;
the residue at position 3 is selected from E and S or is deleted;
the residue at position 4 is selected from Nle, I, V, L, E and S or is deleted;
the residue at position 7 is selected from K and R;
the residue at position 8 is selected from I and H;
the residue at position 9 is selected from Abu and L;
the residue at position 10 is selected from R and K;
the residue at position 11 is Q;
the residue at position 13 is selected from L, A, E, Q, V and G;
the residue at position 14 is selected from V, K, E, L and 2-amino-5-carboxypentanoyl;
the residue at position 15 is selected from S and L;
the residue at position 17 is selected from K, Y and R or is deleted;
the residue at position 18 is selected from D, A, Y, G, V, Q, hQ and L or is deleted;
the residue at position 19 is selected from R and Y or is deleted;
the residue at position 20 is selected from Y, E and R or is deleted;
the residue at position 21 is selected from R, H and E;
the residue at position 22 is selected from R and C;
the residue at position 23 is selected from R, K, hK and P;
the residue at position 28 is Nle;
the residue at position 30 is G;
the residue at position 33 is selected from H, V and R;
the residue at position 35 is selected from F(4-NH₂), F(4-F), F(4-NO₂) and F(4-CH₃);
the residue at position 36 is selected from Q, S and G or is deleted;
the residue at position 37 is selected from S and C or is deleted.

7. An ion channel blocker or pharmaceutically acceptable salt according to claim 1 wherein the Kv1.3 inhibitor component contains no more than 3 deletions, no more than two deletions, or no more than one deletion compared to the sequence of PaT1.

8. An ion channel blocker or pharmaceutically acceptable salt according to claim 1 wherein none of positions 20-33 is deleted.

9. An ion channel blocker or pharmaceutically acceptable salt according to claim 1 wherein, if the Kv1.3 inhibitor component contains more than one deletion, one of those deletions is at position 1.

10. An ion channel blocker or pharmaceutically acceptable salt according to claim 10 wherein, if the Kv1.3 inhibitor component contains more than two deletions, two of those deletions are at positions 1 and 2.

11. An ion channel blocker or pharmaceutically acceptable salt according to claim 1, wherein any substitutions or deletions are made only at the following positions wherein, if different from PaT1:
the residue at position 1 is selected from N and P or is deleted;
the residue at position 2 is Nle, I, V and L;
the residue at position 3 is selected from E and S;
the residue at position 4 is Nle, I, V and L;
the residue at position 8 is I;
the residue at position 10 is R;
the residue at position 11 is Q;
the residue at position 13 is A;
the residue at position 14 is V;
the residue at position 18 is selected from D, Y and A;
the residue at position 19 is R;
the residue at position 20 is Y;
the residue at position 21 is R;
the residue at position 22 is selected from R and C;
the residue at position 23 is R;
the residue at position 28 is Nle;
the residue at position 30 is G;
the residue at position 33 is H;
the residue at position 37 is C.

12. An ion channel blocker or pharmaceutically acceptable salt according to claim 1 wherein the Kv1.3 inhibitor component comprises a maximum of three insertions as compared to the sequence of PaT1.

13. An ion channel blocker or pharmaceutically acceptable salt according to claim 1 wherein the residue at position 1 is not Q.

14. An ion channel blocker or pharmaceutically acceptable salt according to claim 13 wherein the Kv1.3 inhibitor component comprises 1N, 1P or 1*.

15. An ion channel blocker or pharmaceutically acceptable salt according to claim 1 wherein one, two or all three of the residues at positions 2, 4 and 28 of the Kv1.3 inhibitor component are not methionine.

16. An ion channel blocker or pharmaceutically acceptable salt according to claim 15 comprising one of the following combinations of residues:
2*+4Nle, 4I, 4V or 4L;
2Nle+4Nle, 4I, 4V or 4L; or
2I+4Nle, 4I, 4V or 4L.

17. An ion channel blocker or pharmaceutically acceptable salt according to claim 1 wherein the Kv1.3 inhibitor comprises 28Nle.

18. An ion channel blocker or pharmaceutically acceptable salt according to claim 1 wherein the Kv1.3 inhibitor component contains the residues 22S+23I.

19. An ion channel blocker or pharmaceutically acceptable salt according to claim 1 wherein the Kv1.3 inhibitor component contains the residues 13A and/or 18A.

20. An ion channel blocker or pharmaceutically acceptable salt according to claim 19 wherein the Kv1.3 inhibitor component contains one of the following combination of residues:
2Nle+13A;
2Nle+18A;
2Nle+13A+18A;
2Nle+4Nle+13A+28Nle;
2Nle+4Nle+18A+28Nle;
2Nle+4Nle+13A+18A+28Nle;
2Nle+4Nle+28Nle;
2Nle+3E+4Nle+28Nle;
2Nle+3E+4Nle+18A+28Nle;
1P+2Nle+4Nle+28Nle;
1P+2Nle+3E+4Nle+28Nle; or
1P+2Nle+3E+4Nle+18A+28Nle.

21. (canceled)

22. An ion channel blocker or pharmaceutically acceptable salt according to claim 1 which is a fusion protein comprising the Kv1.3 inhibitor component and one or more heterologous polypeptide sequences.

23. An ion channel blocker or pharmaceutically acceptable salt according to claim 22 wherein the Kv1.3 inhibitor component is inserted within a heterologous scaffold polypeptide.

24. An ion channel blocker or pharmaceutically acceptable salt according to claim 1 wherein the ion channel blocker has a maximum length of 200 amino acids, 150 amino acids, 125 amino acids, 100 amino acids, 75 amino acids or 50 amino acids.

25. An ion channel blocker or pharmaceutically acceptable salt according to claim 1 having the formula:
R¹—Z¹—X—Z²—R²
where
R¹ is hydrogen, C₁₋₄ alkyl, acetyl, formyl, benzoyl or trifluoroacetyl;
R² is OH or NH₂
X is a Kv1.3 inhibitor having the variant sequence of PaT1 as described in claim 1, and
Z¹ and Z² are independently sequences of up to 10 amino acid residues;
with the proviso that the ion channel blocker has a maximum length of 50 amino acids.

26-27. (canceled)

28. An ion channel blocker or pharmaceutically acceptable salt according to claim 1 wherein the Kv1.3 inhibitor component comprises one of the sequences:
(SEQ ID NO. 3)
NMDMRCSASVECKQKCLKAIGSIFGKCMNKKCKCYPR
(SEQ ID NO. 5)
MDMRCSASVECKQKCLKAIGSIFGKCMNKKCKCYPR
(SEQ ID NO. 6)
DMRCSASVECKQKCLKAIGSIFGKCMNKKCKCYPR
(SEQ ID NO. 7)
NMDMRCSASVECKQKCLKAIGSIFGKCMNKKCKCYPR
(SEQ ID NO. 8)
NIDMRCSASVECKQKCLKAIGSIFGKCMNKKCKCYPR
(SEQ ID NO. 9)
NMDVRCSASVECKQKCLKAIGSIFGKCMNKKCKCYPR
(SEQ ID NO. 10)
NMDMRCSASVECKQKCKDAIGSIFGKCMNKKCKCYPR
(SEQ ID NO. 12)
NMEMRCSASVECKQKCLKAIGSIFGKCMNKKCKCYPR
(SEQ ID NO. 14)
N[Nle]DMRCSASVECKQKCLKAIGSIFGKCMNKKCKCYPR
(SEQ ID NO. 15)
NMD[Nle]RCSASVECKQKCLKAIGSIFGKCMNKKCKCYPR
(SEQ ID NO. 16)
NMDMRCSASVECKQKCLKAIGSIFGKC[Nle]NKKCKCYPR
(SEQ ID NO. 17)
NMDMRCSASVECKVKCLKAIGSIFGKCMNKKCKCYPR
(SEQ ID NO. 18)
NMDMRCSASVECKQLCLKAIGSIFGKCMNKKCKCYPR
(SEQ ID NO. 19)
NMDMRCSASVECKQKCKKAIGSIFGKCMNKKCKCYPR
(SEQ ID NO. 20)
NMDMRCSASVECKQKCLDAIGSIFGKCMNKKCKCYPR
(SEQ ID NO. 21)
NMDMRCSASVECKQKCLKAIRSIFGKCMNKKCKCYPR
(SEQ ID NO. 22)
NMDMRCSASVECKQKCLKAIESIFGKCMNKKCKCYPR
(SEQ ID NO. 25)
NMDMRCKASVECKQKCLKAIGSIFGKCMNKKCKCYPR
(SEQ ID NO. 26)
NMDMRCSISVECKQKCLKAIGSIFGKCMNKKCKCYPR
(SEQ ID NO. 27)
NMDMRCSASRECKQKCLKAIGSIFGKCMNKKCKCYPR
(SEQ ID NO. 28)
NMDMRCSASVQCKQKCLKAIGSIFGKCMNKKCKCYPR
(SEQ ID NO. 29)
NMDMRCSASVECLQKCLKAIGSIFGKCMNKKCKCYPR
(SEQ ID NO. 30)
NMDMRCSASVECAQKCLKAIGSIFGKCMNKKCKCYPR
(SEQ ID NO. 31)
NMDMRCSASVECKEKCLKAIGSIFGKCMNKKCKCYPR
(SEQ ID NO. 32)
NMDMRCSASVECKLKCLKAIGSIFGKCMNKKCKCYPR
(SEQ ID NO. 33)
NMDMRCSASVECKQKCLKAIHSIFGKCMNKKCKCYPR
(SEQ ID NO. 34)
NMDMRCSASVECKQKCLKAIGSKFGKCMNKKCKCYPR
(SEQ ID NO. 35)
NMDMRCSASVECKQKCLKAIGSRFGKCMNKKCKCYPR
(SEQ ID NO. 36)
NMDMRCSASVECKQKCLKAIGSIFGKCMNGKCKCYPR
(SEQ ID NO. 37)
NMDMRCSASVECKQKCLKAIGSIFGKCMNKKCHCYPR
(SEQ ID NO. 38)
NMDMRCSASVECKQKCLKAIGSIFGKCMNKKCVCYPR
(SEQ ID NO. 39)
N[Nle]D[Nle]RCSASVECKQKCLKAIGSIFGKCMNKKCKCYPR
(SEQ ID NO. 40)
N[Nle]D[Nle]RCSASVECKQKCLKAIGSIFGKC[Nle]NKKCKCYPR
(SEQ ID NO. 41)
P[Nle]D[Nle]RCSASVECKQKCLKAIGSIFGKC[Nle]NKKCKCYPR
(SEQ ID NO. 42)
N[Nle]D[Nle]RCRASVECKQKCLKAIGSIFGKC[Nle]NKKCKCYPR
(SEQ ID NO. 43)
N[Nle]D[Nle]RCSASVECEQKCLKAIGSIFGKC[Nle]NKKCKCYPR
(SEQ ID NO. 44)
N[Nle]D[Nle]RCSASVECQQKCLKAIGSIFGKC[Nle]NKKCKCYPR
(SEQ ID NO. 45)
N[Nle]D[Nle]RCSASVECKKKCLKAIGSIFGKC[Nle]NKKCKCYPR
(SEQ ID NO. 46)
N[Nle]S[Nle]RCSASVECKQKCLKAIGSIFGKC[Nle]NKKCKCYPR
(SEQ ID NO. 47)
N[Nle]D[Nle]RCSHSVECKQKCLKAIGSIFGKC[Nle]NKKCKCYPR
(SEQ ID NO. 48)
N[Nle]D[Nle]RCSASVECKQSCLKAIGSIFGKC[Nle]NKKCKCYPR
(SEQ ID NO. 49)
N[Nle]D[Nle]RCSASVECKQKCKAIGSIFGKC[Nle]NKKCKCYPR
(SEQ ID NO. 50)
NMDMRCSASVECKQKCYKAIGSIFGKCMNKKCKCYPR
(SEQ ID NO. 51)
NMDMRCSASVECKQKCRKAIGSIFGKCMNKKCKCYPR
(SEQ ID NO. 52)
NMDMRCSASVECKQKCLAAIGSIFGKCMNKKCKCYPR
(SEQ ID NO. 53)
NMDMRCSASVECKQKCLYAIGSIFGKCMNKKCKCYPR
(SEQ ID NO. 54)
NMDMRCSASVECKQKCLAIGSIFGKCMNKKCKCYPR
(SEQ ID NO. 55)
NMDMRCSASVECKQKCLKYIGSIFGKCMNKKCKCYPR
(SEQ ID NO. 56)
NMDMRCSASVECKQKCLKRIGSIFGKCMNKKCKCYPR
(SEQ ID NO. 57)
NMDMRCSASVECKQKCLKIGSIFGKCMNKKCKCYPR
(SEQ ID NO. 58)
NMDMRCSASVECKQKCLKAYGSIFGKCMNKKCKCYPR
(SEQ ID NO. 59)
NMDMRCSASVECKQKCLKAEGSIFGKCMNKKCKCYPR
(SEQ ID NO. 60)
NMDMRCSASVECKQKCLKARGSIFGKCMNKKCKCYPR
(SEQ ID NO. 61)
NMDMRCSASVECKQKCLKAGSIFGKCMNKKCKCYPR
(SEQ ID NO. 62)
N[Nle]D[Nle]RCSASVECKQKCLKAIGSPFGKC[Nle]NKKCKCYPR
(SEQ ID NO. 63)
N[Nle]D[Nle]RCSASKECKQKCLKAIGSIFGKC[Nle]NKKCKCYPR
(SEQ ID NO. 64)
H[Nle]D[Nle]RCSASVECKQKCLKAIGSIFGKC[Nle]NKKCKCYPR
(SEQ ID NO. 65)
Y[Nle]D[Nle]RCSASVECKQKCLKAIGSIFGKC[Nle]NKKCKCYPR
(SEQ ID NO. 66)
S[Nle]D[Nle]RCSASVECKQKCLKAIGSIFGKC[Nle]NKKCKCYPR
(SEQ ID NO. 67)
V[Nle]D[Nle]RCSASVECKQKCLKAIGSIFGKC[Nle]NKKCKCYPR
(SEQ ID NO. 68)
S[Nle]D[Nle]RCSA[Abu]VECKQKCLKAIGSIFGKCMNKKCKCYPR
(SEQ ID NO. 69)
S[Nle]D[Nle]RCSASVECKQKCLKAIGSIFG[homo-Lys]CMNKKC
KCYPR
(SEQ ID NO. 70)
S[Nle]D[Nle]RCSASVECKQKCLKAIGSIFGKCMNKKCKC
[F(4-NH₂)]PR
(SEQ ID NO. 71)
S[Nle]D[Nle]RCSASVECGQKCLKAIGSIFGKCMNKKCKCYPR
(SEQ ID NO. 72)
S[Nle]D[Nle]RCSASVECVQKCLKAIGSIFGKCMNKKCKCYPR
(SEQ ID NO. 73)
S[Nle]D[Nle]RCSASVECK[2-Amino-5-carboxypentanoyl]
KCLKAIGSIFGKCMNKKCKCYPR
(SEQ ID NO. 74)
S[Nle]D[Nle]RCSASVECKQKCLKAIGSIFGKCMNKKCKCYQ
(SEQ ID NO. 75)
S[Nle]D[Nle]RCSASVECKQKCLKAIGSIFGKCMNKKCRCYPR
(SEQ ID NO. 76)
S[Nle]DERCSASVECKQKCLKAIGSIFGKCMNKKCKCYPR
(SEQ ID NO. 77)
S[Nle]D[Nle]RCSASVECKQKCLGAIGSIFGKCMNKKCKCYPR
(SEQ ID NO. 78)
S[Nle]D[Nle]RCSASVECKQKCLVAIGSIFGKCMINKKCKCYPR
(SEQ ID NO. 79)
S[Nle]D[Nle]RCSASVECAQSCLKAIGSIFGKCMNKKCKCYPR
(SEQ ID NO. 80)
S[Nle]D[Nle]RCSASVECAQKCLAAIGSIFGKCMNKKCKCYPR
(SEQ ID NO. 81)
S[Nle]D[Nle]RCSASVECAQLCLAAIGSIFGKCMNKKCKCYPR
(SEQ ID NO. 82)
S[Nle]D[Nle]RCSASVECKQKCLKAIGSIFGKC[Nle]NKKCKCYPR
(SEQ ID NO. 83)
P[Nle]D[Nle]RCSASVECKQKCLKAIGCIFGKC[Nle]NKKCKCYPC
(SEQ ID NO. 84)
S[Nle]D[Nle]RCSASVECKEKCLQAIGSIFGKC[Nle]NKKCKCYPR
(SEQ ID NO. 85)
P[Nle]D[Nle]RCSASVECKEKCL[homo-Gln]AIGSIFGKC[Nle]
NKKCKCYPR
(SEQ ID NO. 86)
CSASVECKQKCLKAIGCIFGKC[Nle]NKKCKCYPC
(SEQ ID NO. 87)
S[Nle]D[Nle]RCSASVECKQKCLAAIGCIFGKC[Nle]NKKCKCYPC
(SEQ ID NO. 88)
P[Nle]D[Nle]RCSASVECKQKCLKAIGCIFGKC[Nle]NKKCKCYPC
(SEQ ID NO. 89)
S[Nle]D[Nle]RCSALVECKQKCLKAIGSIFGKCMNKKCKCYPR
(SEQ ID NO. 90)
S[Nle]D[Nle]RCSAVVECKQKCLKAIGSIFGKCMNKKCKC(3)YPR
(SEQ ID NO. 91)
S[Nle]D[Nle]RCSASVECKQKCLKAIGSIFGKCMNKKCKC
[F(4-F)]PR
(SEQ ID NO. 92)
S[Nle]D[Nle]RCSASVECKQKCLKAIGSIFGKCMNKKCKC
[F(4-NO₂)]PR
(SEQ ID NO. 93)
S[Nle]D[Nle]RCSASVECKQKCLKAIGSIFGKCMNKKCKC
[F(4-CH₃)]PR
(SEQ ID NO. 94)
[Nle]D[Nle]RCSASVECKQKCLKAIGSIFGKC[Nle]NKKCKCYPR
(SEQ ID NO. 95)
NID[Nle]RCSASVECKQKCLKAIGSIFGKC[Nle]NKKCKCYPR
(SEQ ID NO. 96)
PIE[Nle]RCSASVECKQKCLKAIGSIFGKC[Nle]NKKCKCYPR
(SEQ ID NO. 97)
P[Nle]E[Nle]RCSASVECKQKCLAAIGSIFGKC[Nle]NKKCKCYPR
(SEQ ID NO. 98)
P[Nle]E[Nle]RCSASVECKQKCLAAIGSIFGKC[Nle]NKKCKCYPR
(SEQ ID NO. 99)
P[Nle]E[Nle]RCSASVECKQKCLLAIGSIFGKC[Nle]NKKCKCYPR
(SEQ ID NO. 100)
PIDERCSASVECKQKCLAAIGSIFGKC[Nle]NKKCKCYPR
(SEQ ID NO. 101)
PIE[Nle]RCSASVECKQKCLAAIGSIFGKC[Nle]NKKCKCYPR
(SEQ ID NO. 102)
P[Nle]D[Nle]RCSASVECAQKCLAAIGSIFGKCMNKKCKCYPR
(SEQ ID NO. 103)
P[Nle]E[Nle]RCSASVECAQKCLAAIGSIFGKC[Nle]NKKCKCYPR
(SEQ ID NO. 104)
CSASVECKQKCLKAIGSIFGKC[Nle]NKKCKCYPR
(SEQ ID NO. 105)
CSASVECKQKCLKAIGSIFGKC[Nle]NKKCKCYPR
(SEQ ID NO. 106)
RCSASVECKQKCLKAIGSIFGKC[Nle]NKKCKCYPR
(SEQ ID NO. 107)
SKCSASVECKQKCLKAIGSIFGKC[Nle]NKKCKCYPR
(SEQ ID NO. 108)
LRCSASVECKQKCLKAIGSIFGKC[Nle]NKKCKCYPR
(SEQ ID NO. 109)
CSASVECKQKCLKAIGSIFGKC[Nle]NKKCKCYPS
(SEQ ID NO. 110)
CSASVECKQKCLKAIGSIFGKC[Nle]NKKCKCYS
(SEQ ID NO. 111)
CSASVECKQKCLKAIGSIFGKC[Nle]NKKCKCYG-NH2
(SEQ ID NO. 112)
CSASVECKQKCLKAIGSIFGKC[Nle]NKKCKCY-NH2
(SEQ ID NO. 113)
RCSASVECKQKCLKAIGSIFGKC[Nle]NKKCKCYPR
(SEQ ID NO. 114)
LRCSASVECKQKCLKAIGSIFGKC[Nle]NKKCKCYPR
(SEQ ID NO. 115)
LRCSASVECKQKCLAAIGSIFGKC[Nle]NKKCKCYPR
(SEQ ID NO. 116)
LRCSASVECKQKCLAAIGSIFGKCMNKKCKCYPR
(SEQ ID NO. 117)
CSASVECKQKCLKAIGSIFGKCMNKKCKCYPR
(SEQ ID NO. 118)
P[Nle]E[Nle]RCSASVECKEKCLAAIGSIFGKC[Nle]NKKCKCYPR
(SEQ ID NO. 119)
P[Nle]E[Nle]RCSASVECKQKCLAAIGSIFGKC[Nle]NKKCKCYPR
(SEQ ID NO. 120)
P[Nle]E[Nle]RCSASVECKQKCLAAIGSIFGKC[Nle]NKKCKCYR
(SEQ ID NO. 121)
[Nle]E[Nle]RCSASVECKQKCLAAIGSIFGKC[Nle]NKKCKCYPR
(SEQ ID NO. 122)
P[Nle]E[Nle]RCSASVECKQKCLAAIGSIFGKC[Nle]NKKCKCYP
RR
(SEQ ID NO. 123)
P[Nle]E[Nle]RCSASVECKQKCLAAIGSIFGKC[Nle]NKKCKCYP
RY
(SEQ ID NO. 124)
P[Nle]E[Nle]RCSASVECKQKCLAAIGSIFGKC[Nle]NKKCKCYP
RL
(SEQ ID NO. 125)
P[Nle]E[Nle]RCSASVECKQKCLAAIGSIFGKC[Nle]NKKCKCYP
RH
(SEQ ID NO. 126)
P[Nle]E[Nle]RCSASVECKQKCLAAIGSIFGKC[Nle]NKKCKCYP
RE
(SEQ ID NO. 127)
P[Nle]E[Nle]RCSASVECKQKCLAAIGSIFGKC[Nle]NKKCKCYPR
KS
(SEQ ID NO. 128)
P[Nle]E[Nle]RCSASVECKQKCLAAIGSIFGKC[Nle]NKKCKCYPR
FE
(SEQ ID NO. 129)
P[Nle]E[Nle]RCSASVECKQKCLAAIGSIFGKC[Nle]NKKCKCYPR
HR
(SEQ ID NO. 130)
P[Nle]E[Nle]RCSASVECKQKCLAAIGSIFGKC[Nle]NKKCKCYPR
AK
(SEQ ID NO. 131)
P[Nle]E[Nle]RCSASVECKQKCLAAIGSIFGKC[Nle]NKKCKCYP-
[(4-amino-5-hydroxypentyl)guanidine]
(SEQ ID NO. 132)
P[Nle]E[Nle]RCSASVECKQKCLAAIGSIFGKC[Nle]NKKCKCYP
R-[4-amino-5-hydroxypentanamide]
(SEQ ID NO. 133)
P[Nle]E[Nle]RCSASVECKQKCLAAIGSIFGKC[Nle]NKKCKCYPR
ST
(SEQ ID NO. 134)
P[Nle]E[Nle]RCSASVECKQKCLAAIGSIFGKC[Nle]NKKCKCYPR
RY
(SEQ ID NO. 135)
P[Nle]E[Nle]RCSSSVECKQKCLAAIGSIFGKC[Nle]NKKCKCYPR
(SEQ ID NO. 136)
P[Nle]E[Nle]RCSLSVECKQKCLAAIGSIFGKC[Nle]NKKCKCYPR
(SEQ ID NO. 137)
P[Nle]E[Nle]RCSAPVECKQKCLAAIGSIFGKC[Nle]NKKCKCYPR
(SEQ ID NO. 138)
P[Nle]E[Nle]RCSASPECKQKCLAAIGSIFGKC[Nle]NKKCKCYPR
(SEQ ID NO. 139)
P[Nle]E[Nle]RCSASQECKQKCLAAIGSIFGKC[Nle]NKKCKCYPR
(SEQ ID NO. 140)
P[Nle]E[Nle]RCSASVECLQKCLAAIGSIFGKC[Nle]NKKCKCYPR
(SEQ ID NO. 141)
P[Nle]E[Nle]RCSASVECKQPCLAAIGSIFGKC[Nle]NKKCKCYPR
(SEQ ID NO. 142)
P[Nle]E[Nle]RCEASVECKQKCLAAIGSIFGKC[Nle]NKKCKCYPR
(SEQ ID NO. 143)
P[Nle]E[Nle]RCFASVECKQKCLAAIGSIFGKC[Nle]NKKCKCYPR
(SEQ ID NO. 144)
P[Nle]E[Nle]RCSYSVECKQKCLAAIGSIFGKC[Nle]NKKCKCYPR
(SEQ ID NO. 145)
P[Nle]E[Nle]RCSAFVECKQKCLAAIGSIFGKC[Nle]NKKCKCYPR.

29. An ion channel blocker or pharmaceutically acceptable salt according to claim 1 comprising or consisting of one of the sequences:
(SEQ ID NO. 3)
NMDMRCSASVECKQKCLKAIGSIFGKCMNKKCKCYPR
(SEQ ID NO. 5)
MDMRCSASVECKQKCLKAIGSIFGKCMNKKCKCYPR
(SEQ ID NO. 6)
DMRCSASVECKQKCLKAIGSIFGKCMNKKCKCYPR
(SEQ ID NO. 7)
NMDMRCSASVECKQKCLKAIGSIFGKCMNKKCKCYPR
(SEQ ID NO. 8)
NIDMRCSASVECKQKCLKAIGSIFGKCMNKKCKCYPR
(SEQ ID NO. 9)
NMDVRCSASVECKQKCLKAIGSIFGKCMNKKCKCYPR
(SEQ ID NO. 10)
NMDMRCSASVECKQKCKDAIGSIFGKCMNKKCKCYPR
(SEQ ID NO. 11)
GGNMDMRCSASVECKQKCLKAIGSIFGKCMNKKCKCYPR
(SEQ ID NO. 12)
NMEMRCSASVECKQKCLKAIGSIFGKCMNKKCKCYPR
(SEQ ID NO. 13)
SGNMDMRCSASVECKQKCLKAIGSIFGKCMNKKCKCYPR
(SEQ ID NO. 14)
N[Nle]DMRCSASVECKQKCLKAIGSIFGKCMNKKCKCYPR
(SEQ ID NO. 15)
NMD[Nle]RCSASVECKQKCLKAIGSIFGKCMNKKCKCYPR
(SEQ ID NO. 16)
NMDMRCSASVECKQKCLKAIGSIFGKC[Nle]NKKCKCYPR
(SEQ ID NO. 17)
NMDMRCSASVECKVKCLKAIGSIFGKCMNKKCKCYPR
(SEQ ID NO. 18)
NMDMRCSASVECKQLCLKAIGSIFGKCMNKKCKCYPR
(SEQ ID NO. 19)
NMDMRCSASVECKQKCKKAIGSIFGKCMNKKCKCYPR
(SEQ ID NO. 20)
NMDMRCSASVECKQKCLDAIGSIFGKCMNKKCKCYPR
(SEQ ID NO. 21)
NMDMRCSASVECKQKCLKAIRSIFGKCMNKKCKCYPR
(SEQ ID NO. 22)
NMDMRCSASVECKQKCLKAIESIFGKCMNKKCKCYPR
(SEQ ID NO. 23)
NMDMRCSASVECKQKCLKAIGSIFGKCMNKKCKCYPRRRTA
(SEQ ID NO. 24)
NMDMRCSASVECKQKCLKAIGSIFGKCMNKKCKCYPRHRRK
(SEQ ID NO. 25)
NMDMRCKASVECKQKCLKAIGSIFGKCMNKKCKCYPR
(SEQ ID NO. 26)
NMDMRCSISVECKQKCLKAIGSIFGKCMNKKCKCYPR
(SEQ ID NO. 27)
NMDMRCSASRECKQKCLKAIGSIFGKCMNKKCKCYPR
(SEQ ID NO. 28)
NMDMRCSASVQCKQKCLKAIGSIFGKCMNKKCKCYPR
(SEQ ID NO. 29)
NMDMRCSASVECLQKCLKAIGSIFGKCMNKKCKCYPR
(SEQ ID NO. 30)
NMDMRCSASVECAQKCLKAIGSIFGKCMNKKCKCYPR
(SEQ ID NO. 31)
NMDMRCSASVECKEKCLKAIGSIFGKCMNKKCKCYPR
(SEQ ID NO. 32)
NMDMRCSASVECKLKCLKAIGSIFGKCMNKKCKCYPR
(SEQ ID NO. 33)
NMDMRCSASVECKQKCLKAIHSIFGKCMNKKCKCYPR
(SEQ ID NO. 34)
NMDMRCSASVECKQKCLKAIGSKFGKCMNKKCKCYPR
(SEQ ID NO. 35)
NMDMRCSASVECKQKCLKAIGSRFGKCMNKKCKCYPR
(SEQ ID NO. 36)
NMDMRCSASVECKQKCLKAIGSIFGKCMNGKCKCYPR
(SEQ ID NO. 37)
NMDMRCSASVECKQKCLKAIGSIFGKCMNKKCHCYPR
(SEQ ID NO. 38)
NMDMRCSASVECKQKCLKAIGSIFGKCMNKKCVCYPR
(SEQ ID NO. 39)
N[Nle]D[Nle]RCSASVECKQKCLKAIGSIFGKCMNKKCKCYPR
(SEQ ID NO. 40)
N[Nle]D[Nle]RCSASVECKQKCLKAIGSIFGKC[Nle]NKKCKCYPR
(SEQ ID NO. 41)
P[Nle]D[Nle]RCSASVECKQKCLKAIGSIFGKC[Nle]NKKCKCYPR
(SEQ ID NO. 42)
N[Nle]D[Nle]RCRASVECKQKCLKAIGSIFGKC[Nle]NKKCKCYPR
(SEQ ID NO. 43)
N[Nle]D[Nle]RCSASVECEQKCLKAIGSIFGKC[Nle]NKKCKCYPR
(SEQ ID NO. 44)
N[Nle]D[Nle]RCSASVECQQKCLKAIGSIFGKC[Nle]NKKCKCYPR
(SEQ ID NO. 45)
N[Nle]D[Nle]RCSASVECKKKCLKAIGSIFGKC[Nle]NKKCKCYPR
(SEQ ID NO. 46)
N[Nle]S[Nle]RCSASVECKQKCLKAIGSIFGKC[Nle]NKKCKCYPR
(SEQ ID NO. 47)
N[Nle]D[Nle]RCSHSVECKQKCLKAIGSIFGKC[Nle]NKKCKCYPR
(SEQ ID NO. 48)
N[Nle]D[Nle]RCSASVECKQSCLKAIGSIFGKC[Nle]NKKCKCYPR
(SEQ ID NO. 49)
N[Nle]D[Nle]RCSASVECKQKCKAIGSIFGKC[Nle]NKKCKCYPR
(SEQ ID NO. 50)
NMDMRCSASVECKQKCYKAIGSIFGKCMNKKCKCYPR
(SEQ ID NO. 51)
NMDMRCSASVECKQKCRKAIGSIFGKCMNKKCKCYPR
(SEQ ID NO. 52)
NMDMRCSASVECKQKCLAAIGSIFGKCMNKKCKCYPR
(SEQ ID NO. 53)
NMDMRCSASVECKQKCLYAIGSIFGKCMNKKCKCYPR
(SEQ ID NO. 54)
NMDMRCSASVECKQKCLAIGSIFGKCMNKKCKCYPR
(SEQ ID NO. 55)
NMDMRCSASVECKQKCLKYIGSIFGKCMNKKCKCYPR
(SEQ ID NO. 56)
NMDMRCSASVECKQKCLKRIGSIFGKCMNKKCKCYPR
(SEQ ID NO. 57)
NMDMRCSASVECKQKCLKIGSIFGKCMNKKCKCYPR
(SEQ ID NO. 58)
NMDMRCSASVECKQKCLKAYGSIFGKCMNKKCKCYPR
(SEQ ID NO. 59)
NMDMRCSASVECKQKCLKAEGSIFGKCMNKKCKCYPR
(SEQ ID NO. 60)
NMDMRCSASVECKQKCLKARGSIFGKCMNKKCKCYPR
(SEQ ID NO. 61)
NMDMRCSASVECKQKCLKAGSIFGKCMNKKCKCYPR
(SEQ ID NO. 62)
N[Nle]D[Nle]RCSASVECKQKCLKAIGSPFGKC[Nle]NKKCKCYPR
(SEQ ID NO. 63)
N[Nle]D[Nle]RCSASKECKQKCLKAIGSIFGKC[Nle]NKKCKCYPR
(SEQ ID NO. 64)
H[Nle]D[Nle]RCSASVECKQKCLKAIGSIFGKC[Nle]NKKCKCYPR
(SEQ ID NO. 65)
Y[Nle]D[Nle]RCSASVECKQKCLKAIGSIFGKC[Nle]NKKCKCYPR
(SEQ ID NO. 66)
S[Nle]D[Nle]RCSASVECKQKCLKAIGSIFGKC[Nle]NKKCKCYPR
(SEQ ID NO. 67)
V[Nle]D[Nle]RCSASVECKQKCLKAIGSIFGKC[Nle]NKKCKCYPR
(SEQ ID NO. 68)
S[Nle]D[Nle]RCSA[Abu]VECKQKCLKAIGSIFGKCMNKKCKCYPR
(SEQ ID NO. 69)
S[Nle]D[Nle]RCSASVECKQKCLKAIGSIFG[homo-Lys]CMNKKCKC
YPR
(SEQ ID NO. 70)
S[Nle]D[Nle]RCSASVECKQKCLKAIGSIFGKCMNKKCKC
[F(4-NH2)]PR
(SEQ ID NO. 71)
S[Nle]D[Nle]RCSASVECGQKCLKAIGSIFGKCMNKKCKCYPR
(SEQ ID NO. 72)
S[Nle]D[Nle]RCSASVECVQKCLKAIGSIFGKCMNKKCKCYPR
(SEQ ID NO. 73)
S[Nle]D[Nle]RCSASVECK[2-Amino-5-carboxypentanoyl]
KCLKAIGSIFGKCMNKKCKCYPR
(SEQ ID NO. 74)
S[Nle]D[Nle]RCSASVECKQKCLKAIGSIFGKCMNKKCKCYQ
(SEQ ID NO. 75)
S[Nle]D[Nle]RCSASVECKQKCLKAIGSIFGKCMNKKCRCYPR
(SEQ ID NO. 76)
S[Nle]DERCSASVECKQKCLKAIGSIFGKCMNKKCKCYPR
(SEQ ID NO. 77)
S[Nle]D[Nle]RCSASVECKQKCLGAIGSIFGKCMNKKCKCYPR
(SEQ ID NO. 78)
S[Nle]D[Nle]RCSASVECKQKCLVAIGSIFGKCMNKKCKCYPR
(SEQ ID NO. 79)
S[Nle]D[Nle]RCSASVECAQSCLKAIGSIFGKCMNKKCKCYPR
(SEQ ID NO. 80)
S[Nle]D[Nle]RCSASVECAQKCLAAIGSIFGKCMNKKCKCYPR
(SEQ ID NO. 81)
S[Nle]D[Nle]RCSASVECAQLCLAAIGSIFGKCMNKKCKCYPR
(SEQ ID NO. 82)
S[Nle]D[Nle]RCSASVECKQKCLKAIGSIFGKC[Nle]NKKCKCYPR
(SEQ ID NO. 83)
P[Nle]D[Nle]RCSASVECKQKCLKAIGCIFGKC[Nle]NKKCKCYPC
(SEQ ID NO. 84)
S[Nle]D[Nle]RCSASVECKEKCLQAIGSIFGKC[Nle]NKKCKCYPR
(SEQ ID NO. 85)
P[Nle]D[Nle]RCSASVECKEKCL[homo-Gln]AIGSIFGKC[Nle]
NKKCKCYPR
(SEQ ID NO. 86)
CSASVECKQKCLKAIGCIFGKC[Nle]NKKCKCYPC
(SEQ ID NO. 87)
S[Nle]D[Nle]RCSASVECKQKCLAAIGCIFGKC[Nle]NKKCKCYPC
(SEQ ID NO. 88)
P[Nle]D[Nle]RCSASVECKQKCLKAIGCIFGKC[Nle]NKKCKCYPC
(SEQ ID NO. 89)
S[Nle]D[Nle]RCSALVECKQKCLKAIGSIFGKCMNKKCKCYPR
(SEQ ID NO. 90)
S[Nle]D[Nle]RCSAVVECKQKCLKAIGSIFGKCMNKKCKCYPR
(SEQ ID NO. 91)
S[Nle]D[Nle]RCSASVECKQKCLKAIGSIFGKCMNKKCKC[F(4-F)]
PR
(SEQ ID NO. 92)
S[Nle]D[Nle]RCSASVECKQKCLKAIGSIFGKCMNKKCKC
[F(4-NO₂)]PR
(SEQ ID NO. 93)
S[Nle]D[Nle]RCSASVECKQKCLKAIGSIFGKCMNKKCKC
[F(4-CH₃)]PR
(SEQ ID NO. 94)
[Nle]D[Nle]RCSASVECKQKCLKAIGSIFGKC[Nle]NKKCKCYPR
(SEQ ID NO. 95)
NID[Nle]RCSASVECKQKCLKAIGSIFGKC[Nle]NKKCKCYPR
(SEQ ID NO. 96)
PIE[Nle]RCSASVECKQKCLKAIGSIFGKC[Nle]NKKCKCYPR
(SEQ ID NO. 97)
P[Nle]E[Nle]RCSASVECKQKCLAAIGSIFGKC[Nle]NKKCKCYPR
(SEQ ID NO. 98)
P[Nle]E[Nle]RCSASVECKQKCLAAIGSIFGKC[Nle]NKKCKCYPR
(SEQ ID NO. 99)
P[Nle]E[Nle]RCSASVECKQKCLLAIGSIFGKC[Nle]NKKCKCYPR
(SEQ ID NO. 100)
PIDERCSASVECKQKCLAAIGSIFGKC[Nle]NKKCKCYPR
(SEQ ID NO. 101)
PIE[Nle]RCSASVECKQKCLAAIGSIFGKC[Nle]NKKCKCYPR
(SEQ ID NO. 102)
P[Nle]D[Nle]RCSASVECAQKCLAAIGSIFGKCMNKKCKCYPR
(SEQ ID NO. 103)
P[Nle]E[Nle]RCSASVECAQKCLAAIGSIFGKC[Nle]NKKCKCYPR
(SEQ ID NO. 104)
CSASVECKQKCLKAIGSIFGKC[Nle]NKKCKCYPR
(SEQ ID NO. 105)
CSASVECKQKCLKAIGSIFGKC[Nle]NKKCKCYPR
(SEQ ID NO. 106)
RCSASVECKQKCLKAIGSIFGKC[Nle]NKKCKCYPR
(SEQ ID NO. 107)
SKCSASVECKQKCLKAIGSIFGKC[Nle]NKKCKCYPR
(SEQ ID NO. 108)
LRCSASVECKQKCLKAIGSIFGKC[Nle]NKKCKCYPR
(SEQ ID NO. 109)
CSASVECKQKCLKAIGSIFGKC[Nle]NKKCKCYPS
(SEQ ID NO. 110)
CSASVECKQKCLKAIGSIFGKC[Nle]NKKCKCYS
(SEQ ID NO. 111)
CSASVECKQKCLKAIGSIFGKC[Nle]NKKCKCYG-NH2
(SEQ ID NO. 112)
CSASVECKQKCLKAIGSIFGKC[Nle]NKKCKCY-NH2
(SEQ ID NO. 113)
RCSASVECKQKCLKAIGSIFGKC[Nle]NKKCKCYPR
(SEQ ID NO. 114)
LRCSASVECKQKCLKAIGSIFGKC[Nle]NKKCKCYPR
(SEQ ID NO. 115)
LRCSASVECKQKCLAAIGSIFGKC[Nle]NKKCKCYPR
(SEQ ID NO. 116)
LRCSASVECKQKCLAAIGSIFGKCMNKKCKCYPR
(SEQ ID NO. 117)
CSASVECKQKCLKAIGSIFGKCMNKKCKCYPR
(SEQ ID NO. 118)
P[Nle]E[Nle]RCSASVECKEKCLAAIGSIFGKC[Nle]NKKCKCYPR
(SEQ ID NO. 119)
P[Nle]E[Nle]RCSASVECKQKCLAAIGSIFGKC[Nle]NKKCKCYPR
(SEQ ID NO. 120)
P[Nle]E[Nle]RCSASVECKQKCLAAIGSIFGKC[Nle]NKKCKCYR
(SEQ ID NO. 121)
[Nle]E[Nle]RCSASVECKQKCLAAIGSIFGKC[Nle]NKKCKCYPR
(SEQ ID NO. 122)
P[Nle]E[Nle]RCSASVECKQKCLAAIGSIFGKC[Nle]NKKCKCYPRR
(SEQ ID NO. 123)
P[Nle]E[Nle]RCSASVECKQKCLAAIGSIFGKC[Nle]NKKCKCYPRY
(SEQ ID NO. 124)
P[Nle]E[Nle]RCSASVECKQKCLAAIGSIFGKC[Nle]NKKCKCYPRL
(SEQ ID NO. 125)
P[Nle]E[Nle]RCSASVECKQKCLAAIGSIFGKC[Nle]NKKCKCYPRH
(SEQ ID NO. 126)
P[Nle]E[Nle]RCSASVECKQKCLAAIGSIFGKC[Nle]NKKCKCYPRE
(SEQ ID NO. 127)
P[Nle]E[Nle]RCSASVECKQKCLAAIGSIFGKC[Nle]NKKCKCYPRKS
(SEQ ID NO. 128)
P[Nle]E[Nle]RCSASVECKQKCLAAIGSIFGKC[Nle]NKKCKCYPRFE
(SEQ ID NO. 129)
P[Nle]E[Nle]RCSASVECKQKCLAAIGSIFGKC[Nle]NKKCKCYPRHR
(SEQ ID NO. 130)
P[Nle]E[Nle]RCSASVECKQKCLAAIGSIFGKC[Nle]NKKCKCYPRAK
(SEQ ID NO. 131)
P[Nle]E[Nle]RCSASVECKQKCLAAIGSIFGKC[Nle]NKKCKCYP-
[(4-amino-5-hydroxypentyl)guanidine]
(SEQ ID NO. 132)
P[Nle]E[Nle]RCSASVECKQKCLAAIGSIFGKC[Nle]NKKCKCYPR-
[4-amino-5-hydroxypentanamide]
(SEQ ID NO. 133)
P[Nle]E[Nle]RCSASVECKQKCLAAIGSIFGKC[Nle]NKKCKCYPRST
(SEQ ID NO. 1347)
P[Nle]E[Nle]RCSASVECKQKCLAAIGSIFGKC[Nle]NKKCKCYPRRY
(SEQ ID NO. 135)
P[Nle]E[Nle]RCSSSVECKQKCLAAIGSIFGKC[Nle]NKKCKCYPR
(SEQ ID NO. 136)
P[Nle]E[Nle]RCSLSVECKQKCLAAIGSIFGKC[Nle]NKKCKCYPR
(SEQ ID NO. 137)
P[Nle]E[Nle]RCSAPVECKQKCLAAIGSIFGKC[Nle]NKKCKCYPR
(SEQ ID NO. 138)
P[Nle]E[Nle]RCSASPECKQKCLAAIGSIFGKC[Nle]NKKCKCYPR
(SEQ ID NO. 139)
P[Nle]E[Nle]RCSASQECKQKCLAAIGSIFGKC[Nle]NKKCKCYPR
(SEQ ID NO. 140)
P[Nle]E[Nle]RCSASVECLQKCLAAIGSIFGKC[Nle]NKKCKCYPR
(SEQ ID NO. 141)
P[Nle]E[Nle]RCSASVECKQPCLAAIGSIFGKC[Nle]NKKCKCYPR
(SEQ ID NO. 142)
P[Nle]E[Nle]RCEASVECKQKCLAAIGSIFGKC[Nle]NKKCKCYPR
(SEQ ID NO. 143)
P[Nle]E[Nle]RCFASVECKQKCLAAIGSIFGKC[Nle]NKKCKCYPR
(SEQ ID NO. 144)
P[Nle]E[Nle]RCSYSVECKQKCLAAIGSIFGKC[Nle]NKKCKCYPR
(SEQ ID NO. 145)
P[Nle]E[Nle]RCSAFVECKQKCLAAIGSIFGKC[Nle]NKKCKCYPR.

30. An ion channel blocker or pharmaceutically acceptable salt according to claim 1 selected from:

| | |
|---|---|
| H-NMDMRCSASVECKQKCLKAIGSIFGKCMNKKCKCYPR-OH (SEQ ID | (Cpd. 3) |
| NO. 3) | |
| H-MDMRCSASVECKQKCLKAIGSIFGKCMNKKCKCYPR-OH (SEQ ID | (Cpd. 5) |
| NO. 5) | |
| H-DMRCSASVECKQKCLKAIGSIFGKCMNKKCKCYPR-OH (SEQ ID | (Cpd. 6) |
| NO. 6) | |
| H-NMDMRCSASVECKQKCLKAIGSIFGKCMNKKCKCYPR-NH₂ (SEQ | (Cpd. 7) |
| ID NO. 7) | |
| H-NIDMRCSASVECKQKCLKAIGSIFGKCMNKKCKCYPR-OH (SEQ ID | (Cpd. 8) |
| NO. 8) | |
| H-NMDVRCSASVECKQKCLKAIGSIFGKCMNKKCKCYPR-OH (SEQ ID | (Cpd. 9) |
| NO. 9) | |
| H-NMDMRCSASVECKQKCKDAIGSIFGKCMNKKCKCYPR-OH (SEQ | (Cpd. 10) |
| ID NO. 10) | |
| H-GGNMDMRCSASVECKQKCLKAIGSIFGKCMNKKCKCYPR-OH | (Cpd. 11) |
| (SEQ ID NO. 11) | |
| H-NMEMRCSASVECKQKCLKAIGSIFGKCMNKKCKCYPR-OH (SEQ ID | (Cpd. 12) |
| NO. 12) | |
| H-SGNMDMRCSASVECKQKCLKAIGSIFGKCMNKKCKCYPR-OH | (Cpd. 13) |
| (SEQ ID NO. 13) | |
| H-N[Nle]DMRCSASVECKQKCLKAIGSIFGKCMNKKCKCYPR-NH₂ | (Cpd. 14) |
| (SEQ ID NO. 14) | |
| H-NMD[Nle]RCSASVECKQKCLKAIGSIFGKCMNKKCKCYPR-NH₂ | (Cpd. 15) |
| (SEQ ID NO. 15) | |
| H-NMDMRCSASVECKQKCLKAIGSIFGKC[Nle]NKKCKCYPR-NH₂ | (Cpd. 16) |
| (SEQ ID NO. 16) | |
| H-NMDMRCSASVECKVKCLKAIGSIFGKCMNKKCKCYPR-NH₂ (SEQ | (Cpd. 17) |
| ID NO. 17) | |
| H-NMDMRCSASVECKQLCLKAIGSIFGKCMNKKCKCYPR-NH₂ (SEQ | (Cpd. 18) |
| ID NO. 18) | |
| H-NMDMRCSASVECKQKCKKAIGSIFGKCMNKKCKCYPR-NH₂ (SEQ | (Cpd. 19) |
| ID NO. 19) | |
| H-NMDMRCSASVECKQKCLDAIGSIFGKCMNKKCKCYPR-NH₂ (SEQ | (Cpd. 20) |
| ID NO. 20) | |
| H-NMDMRCSASVECKQKCLKAIRSIFGKCMNKKCKCYPR-NH₂ (SEQ | (Cpd. 21) |
| ID NO. 21) | |
| H-NMDMRCSASVECKQKCLKAIESIFGKCMNKKCKCYPR-NH₂ (SEQ | (Cpd. 22) |
| ID NO. 22) | |
| H-NMDMRCSASVECKQKCLKAIGSIFGKCMNKKCKCYPRRRTA-NH₂ | (Cpd. 23) |
| (SEQ ID NO. 23) | |
| H-NMDMRCSASVECKQKCLKAIGSIFGKCMNKKCKCYPRHRRK-NH₂ | (Cpd. 24) |
| (SEQ ID NO. 24) | |
| H-NMDMRCKASVECKQKCLKAIGSIFGKCMNKKCKCYPR-NH₂ (SEQ | (Cpd. 25) |
| ID NO. 25) | |
| H-NMDMRCSISVECKQKCLKAIGSIFGKCMNKKCKCYPR-NH₂ (SEQ ID | (Cpd. 26) |
| NO. 26) | |
| H-NMDMRCSASRECKQKCLKAIGSIFGKCMNKKCKCYPR-NH₂ (SEQ | (Cpd. 27) |
| ID NO. 27) | |
| H-NMDMRCSASVQCKQKCLKAIGSIFGKCMNKKCKCYPR-NH₂ (SEQ | (Cpd. 28) |
| ID NO. 28) | |
| H-NMDMRCSASVECLQKCLKAIGSIFGKCMNKKCKCYPR-NH₂ (SEQ | (Cpd. 29) |
| ID NO. 29) | |
| H-NMDMRCSASVECAQKCLKAIGSIFGKCMNKKCKCYPR-NH₂ (SEQ | (Cpd. 30) |
| ID NO. 30) | |
| H-NMDMRCSASVECKEKCLKAIGSIFGKCMNKKCKCYPR-NH₂ (SEQ | (Cpd. 31) |
| ID NO. 31) | |
| H-NMDMRCSASVECKLKCLKAIGSIFGKCMNKKCKCYPR-NH₂ (SEQ ID | (Cpd. 32) |
| NO. 32) | |
| H-NMDMRCSASVECKQKCLKAIHSIFGKCMNKKCKCYPR-NH₂ (SEQ | (Cpd. 33) |
| ID NO. 33) | |
| H-NMDMRCSASVECKQKCLKAIGSKFGKCMNKKCKCYPR-NH₂ (SEQ | (Cpd. 34) |
| ID NO. 34) | |
| H-NMDMRCSASVECKQKCLKAIGSRFGKCMNKKCKCYPR-NH₂ (SEQ | (Cpd. 35) |
| ID NO. 35) | |
| H-NMDMRCSASVECKQKCLKAIGSIFGKCMNGKCKCYPR-NH₂ (SEQ | (Cpd. 36) |
| ID NO. 36) | |
| H-NMDMRCSASVECKQKCLKAIGSIFGKCMNKKCHCYPR-NH₂ (SEQ | (Cpd. 37) |
| ID NO. 37) | |
| H-NMDMRCSASVECKQKCLKAIGSIFGKCMNKKCVCYPR-NH₂ (SEQ | (Cpd. 38) |
| ID NO. 38) | |
| H-N[Nle]D[Nle]RCSASVECKQKCLKAIGSIFGKCMNKKCKCYPR-NH₂ | (Cpd. 39) |
| (SEQ ID NO. 39) | |
| H-N[Nle]D[Nle]RCSASVECKQKCLKAIGSIFGKC[Nle]NKKCKCYPR-NH₂ | (Cpd. 40) |
| (SEQ ID NO. 40) | |
| H-P[Nle]D[Nle]RCSASVECKQKCLKAIGSIFGKC[Nle]NKKCKCYPR-NH₂ | (Cpd. 41) |
| (SEQ ID NO. 41) | |
| H-N[Nle]D[Nle]RCRASVECKQKCLKAIGSIFGKC[Nle]NKKCKCYPR-NH₂ | (Cpd. 42) |
| (SEQ ID NO. 42) | |
| H-N[Nle]D[Nle]RCSASVECEQKCLKAIGSIFGKC[Nle]NKKCKCYPR-NH₂ | (Cpd. 43) |
| (SEQ ID NO. 43) | |
| H-N[Nle]D[Nle]RCSASVECQQKCLKAIGSIFGKC[Nle]NKKCKCYPR-NH₂ | (Cpd. 44) |
| (SEQ ID NO. 44) | |
| H-N[Nle]D[Nle]RCSASVECKKKCLKAIGSIFGKC[Nle]NKKCKCYPR-NH₂ | (Cpd. 45) |
| (SEQ ID NO. 45) | |
| H-N[Nle]S[Nle]RCSASVECKQKCLKAIGSIFGKC[Nle]NKKCKCYPR-NH₂ | (Cpd. 46) |
| (SEQ ID NO. 46) | |
| H-N[Nle]D[Nle]RCSHSVECKQKCLKAIGSIFGKC[Nle]NKKCKCYPR-NH₂ | (Cpd. 47) |
| (SEQ ID NO. 47) | |
| H-N[Nle]D[Nle]RCSASVECKQSCLKAIGSIFGKC[Nle]NKKCKCYPR-NH₂ | (Cpd. 48) |
| (SEQ ID NO. 48) | |
| H-N[Nle]D[Nle]RCSASVECKQKCKAIGSIFGKC[Nle]NKKCKCYPR-NH₂ | (Cpd. 49) |
| (SEQ ID NO. 49) | |
| H-NMDMRCSASVECKQKCYKAIGSIFGKCMNKKCKCYPR-NH₂ (SEQ | (Cpd. 50) |
| ID NO. 50) | |
| H-NMDMRCSASVECKQKCRKAIGSIFGKCMNKKCKCYPR-NH₂ (SEQ | (Cpd. 51) |
| ID NO. 51) | |
| H-NMDMRCSASVECKQKCLAAIGSIFGKCMNKKCKCYPR-NH₂ (SEQ | (Cpd. 52) |
| ID NO. 52) | |
| H-NMDMRCSASVECKQKCLYAIGSIFGKCMNKKCKCYPR-NH₂ (SEQ | (Cpd. 53) |
| ID NO. 53) | |
| H-NMDMRCSASVECKQKCLAIGSIFGKCMNKKCKCYPR-NH₂ (SEQ ID | (Cpd. 54) |
| NO. 54) | |
| H-NMDMRCSASVECKQKCLKYIGSIFGKCMNKKCKCYPR-NH₂ (SEQ | (Cpd. 55) |
| ID NO. 55) | |
| H-NMDMRCSASVECKQKCLKRIGSIFGKCMNKKCKCYPR-NH₂ (SEQ | (Cpd. 56) |
| ID NO. 56) | |
| H-NMDMRCSASVECKQKCLKIGSIFGKCMNKKCKCYPR-NH₂ (SEQ ID | (Cpd. 57) |
| NO. 57) | |
| H-NMDMRCSASVECKQKCLKAYGSIFGKCMNKKCKCYPR-NH₂ (SEQ | (Cpd. 58) |
| ID NO. 58) | |
| H-NMDMRCSASVECKQKCLKAEGSIFGKCMNKKCKCYPR-NH₂ (SEQ | (Cpd. 59) |
| ID NO. 59) | |
| H-NMDMRCSASVECKQKCLKARGSIFGKCMNKKCKCYPR-NH₂ (SEQ | (Cpd. 60) |
| ID NO. 60) | |
| H-NMDMRCSASVECKQKCLKAGSIFGKCMNKKCKCYPR-NH₂ (SEQ ID | (Cpd. 61) |
| NO. 61) | |
| H-N[Nle]D[Nle]RCSASVECKQKCLKAIGSPFGKC[Nle]NKKCKCYPR- | (Cpd. 62) |
| NH₂ (SEQ ID NO. 62) | |
| H-N[Nle]D[Nle]RCSASKECKQKCLKAIGSIFGKC[Nle]NKKCKCYPR-NH₂ | (Cpd. 63) |
| (SEQ ID NO. 63) | |
| H-H[Nle]D[Nle]RCSASVECKQKCLKAIGSIFGKC[Nle]NKKCKCYPR-NH₂ | (Cpd. 64) |
| (SEQ ID NO. 64) | |
| H-Y[Nle]D[Nle]RCSASVECKQKCLKAIGSIFGKC[Nle]NKKCKCYPR-NH₂ | (Cpd. 65) |
| (SEQ ID NO. 65) | |
| H-S[Nle]D[Nle]RCSASVECKQKCLKAIGSIFGKC[Nle]NKKCKCYPR-NH₂ | (Cpd. 66) |
| (SEQ ID NO. 66) | |
| H-V[Nle]D[Nle]RCSASVECKQKCLKAIGSIFGKC[Nle]NKKCKCYPR-NH₂ | (Cpd. 67) |
| (SEQ ID NO. 67) | |
| H-S[Nle]D[Nle]RCSA[Abu]VECKQKCLKAIGSIFGKCMNKKCKCYPR- | (Cpd. 68) |
| NH₂ (SEQ ID NO. 68) | |
| H-S[Nle]D[Nle]RCSASVECKQKCLKAIGSIFG[homo- | (Cpd. 69) |
| Lys]CMNKKCKCYPR-NH2 (SEQ ID NO. 69) | |
| H-S[Nle]D[Nle]RCSASVECKQKCLKAIGSIFGKCMNKKCKC[F(4- | (Cpd. 70) |
| NH₂)]PR-NH₂ (SEQ ID NO. 70) | |
| H-S[Nle]D[Nle]RCSASVECGQKCLKAIGSIFGKCMNKKCKCYPR-NH₂ | (Cpd. 71) |
| (SEQ ID NO. 71) | |
| H-S[Nle]D[Nle]RCSASVECVQKCLKAIGSIFGKCMNKKCKCYPR-NH₂ | (Cpd. 72) |
| (SEQ ID NO. 72) | |
| H-S[Nle]D[Nle]RCSASVECK[2-Amino-5-carboxypentanoyl] | (Cpd. 73) |
| KCLKAIGSIFGKCMNKKCKCYPR-NH₂ (SEQ ID NO. 73) | |
| H-S[Nle]D[Nle]RCSASVECKQKCLKAIGSIFGKCMNKKCKCYQ-NH₂ | (Cpd. 74) |
| (SEQ ID NO. 74) | |
| H-S[Nle]D[Nle]RCSASVECKQKCLKAIGSIFGKCMNKKCRCYPR-NH₂ | (Cpd. 75) |
| (SEQ ID NO. 75) | |
| H-S[Nle]DERCSASVECKQKCLKAIGSIFGKCMNKKCKCYPR-NH₂ (SEQ | (Cpd. 76) |
| ID NO. 76) | |
| H-S[Nle]D[Nle]RCSASVECKQKCLGAIGSIFGKCMNKKCKCYPR-NH₂ | (Cpd. 77) |
| (SEQ ID NO. 77) | |
| H-S[Nle]D[Nle]RCSASVECKQKCLVAIGSIFGKCMNKKCKCYPR-NH₂ | (Cpd. 78) |
| (SEQ ID NO. 78) | |
| H-S[Nle]D[Nle]RCSASVECAQSCLKAIGSIFGKCMNKKCKCYPR-NH₂ | (Cpd. 79) |
| (SEQ ID NO. 79) | |
| H-S[Nle]D[Nle]RCSASVECAQKCLAAIGSIFGKCMNKKCKCYPR-NH₂ | (Cpd. 80) |
| (SEQ ID NO. 80) | |
| H-S[Nle]D[Nle]RCSASVECAQLCLAAIGSIFGKCMNKKCKCYPR-NH₂ | (Cpd. 81) |
| (SEQ ID NO. 81) | |
| H-S[Nle]D[Nle]RCSASVECKQKCLKAIGSIFGKC[Nle]NKKCKCYPR-OH | (Cpd. 82) |
| (SEQ ID NO. 82) | |
| H-P[Nle]D[Nle]RCSASVECKQKCLKAIGCIFGKC[Nle]NKKCKCYPC-NH₂ | (Cpd. 83) |
| (SEQ ID NO. 83) | |
| H-S[Nle]D[Nle]RCSASVECKEKCLQAIGSIFGKC[Nle]NKKCKCYPR-NH₂ | (Cpd. 84) |
| (SEQ ID NO. 84) | |
| H-P[Nle]D[Nle]RCSASVECKEKCL[homo-Gln]AIGSIFGKC[Nle] | (Cpd. 85) |
| NKKCKCYPR-NH₂ (SEQ ID NO. 85) | |
| H-CSASVECKQKCLKAIGCIFGKC[Nle]NKKCKCYPC-NH₂ (SEQ ID NO. | (Cpd. 86) |
| 86) | |
| H-S[Nle]D[Nle]RCSASVECKQKCLAAIGCIFGKC[Nle]NKKCKCYPC-NH₂ | (Cpd. 87) |
| (SEQ ID NO. 87) | |
| H-P[Nle]D[Nle]RCSASVECKQKCLKAIGCIFGKC[Nle]NKKCKCYPC-OH | (Cpd. 88) |
| (SEQ ID NO. 88) | |
| H-S[Nle]D[Nle]RCSALVECKQKCLKAIGSIFGKCMNKKCKCYPR-NH₂ | (Cpd. 89) |
| (SEQ ID NO. 89) | |
| H-S[Nle]D[Nle]RCSAVVECKQKCLKAIGSIFGKCMNKKCKCYPR-NH₂ | (Cpd. 90) |
| (SEQ ID NO. 90) | |
| H-S[Nle]D[Nle]RCSASVECKQKCLKAIGSIFGKCMNKKCKC[F(4-F)]PR- | (Cpd. 91) |
| NH₂ (SEQ ID NO. 91) | |
| H-S[Nle]D[Nle]RCSASVECKQKCLKAIGSIFGKCMNKKCKC[F(4-NO₂)] | (Cpd. 92) |
| PR-NH₂ (SEQ ID NO. 92) | |
| H-S[Nle]D[Nle]RCSASVECKQKCLKAIGSIFGKCMNKKCKC[F(4-CH₃)] | (Cpd. 93) |
| PR-NH₂ (SEQ ID NO. 93) | |
| H-[Nle]D[Nle]RCSASVECKQKCLKAIGSIFGKC[Nle]NKKCKCYPR-OH | (Cpd. 94) |
| (SEQ ID NO. 94) | |
| H-NID[Nle]RCSASVECKQKCLKAIGSIFGKC[Nle]NKKCKCYPR-OH | (Cpd. 95) |
| (SEQ ID NO. 95) | |
| H-PIE[Nle]RCSASVECKQKCLKAIGSIFGKC[Nle]NKKCKCYPR-OH | (Cpd. 96) |
| (SEQ ID NO. 96) | |
| H-P[Nle]E[Nle]RCSASVECKQKCLAAIGSIFGKC[Nle]NKKCKCYPR-OH | (Cpd. 97) |
| (SEQ ID NO. 97) | |
| H-P[Nle]E[Nle]RCSASVECKQKCLAAIGSIFGKC[Nle]NKKCKCYPR-NH₂ | (Cpd. 98) |
| (SEQ ID NO. 98) | |
| H-P[Nle]E[Nle]RCSASVECKQKCLLAIGSIFGKC[Nle]NKKCKCYPR-OH | (Cpd. 99) |
| (SEQ ID NO. 99) | |
| H-PIDERCSASVECKQKCLAAIGSIFGKC[Nle]NKKCKCYPR-OH (SEQ | (Cpd. 100) |
| ID NO. 100) | |
| H-PIE[Nle]RCSASVECKQKCLAAIGSIFGKC[Nle]NKKCKCYPR-OH | (Cpd. 101) |
| (SEQ ID NO. 101) | |
| H-P[Nle]D[Nle]RCSASVECAQKCLAAIGSIFGKCMNKKCKCYPR-OH | (Cpd. 102) |
| (SEQ ID NO. 102) | |
| H-P[Nle]E[Nle]RCSASVECAQKCLAAIGSIFGKC[Nle]NKKCKCYPR-OH | (Cpd. 103) |
| (SEQ ID NO. 103) | |
| H-CSASVECKQKCLKAIGSIFGKC[Nle]NKKCKCYPR-NH₂ (SEQ ID NO. | (Cpd. 104) |
| 104) | |
| Ac-CSASVECKQKCLKAIGSIFGKC[Nle]NKKCKCYPR-NH₂ (SEQ ID | (Cpd. 105) |
| NO. 105) | |
| H-RCSASVECKQKCLKAIGSIFGKC[Nle]NKKCKCYPR-NH₂ (SEQ ID | (Cpd. 106) |
| NO. 106) | |
| Ac-SKCSASVECKQKCLKAIGSIFGKC[Nle]NKKCKCYPR-NH₂ (SEQ ID | (Cpd. 107) |
| NO. 107) | |
| Ac-LRCSASVECKQKCLKAIGSIFGKC[Nle]NKKCKCYPR-NH₂ (SEQ ID | (Cpd. 108) |
| NO. 108) | |
| Ac-CSASVECKQKCLKAIGSIFGKC[Nle]NKKCKCYPS-NH₂ (SEQ ID | (Cpd. 109) |
| NO. 109) | |
| Ac-CSASVECKQKCLKAIGSIFGKC[Nle]NKKCKCYS-NH₂ (SEQ ID NO. | (Cpd. 110) |
| 110) | |
| Ac-CSASVECKQKCLKAIGSIFGKC[Nle]NKKCKCYG-NH₂ (SEQ ID NO. | (Cpd. 111) |
| 111) | |
| Ac-CSASVECKQKCLKAIGSIFGKC[Nle]NKKCKCY-NH₂ (SEQ ID NO. | (Cpd. 112) |
| 112) | |
| Ac-RCSASVECKQKCLKAIGSIFGKC[Nle]NKKCKCYPR-OH (SEQ ID | (Cpd. 113) |
| NO. 113) | |
| H-LRCSASVECKQKCLKAIGSIFGKC[Nle]NKKCKCYPR-OH (SEQ ID | (Cpd. 114) |
| NO. 114) | |
| Ac-LRCSASVECKQKCLAAIGSIFGKC[Nle]NKKCKCYPR-OH (SEQ ID | (Cpd. 115) |
| NO. 115) | |
| Ac-LRCSASVECKQKCLAAIGSIFGKCMNKKCKCYPR-OH (SEQ ID NO. | (Cpd. 116) |
| 116) | |
| H-CSASVECKQKCLKAIGSIFGKCMNKKCKCYPR-OH (SEQ ID NO. | (Cpd. 117) |
| 117) | |
| H-P[Nle]E[Nle]RCSASVECKEKCLAAIGSIFGKC[Nle]NKKCKCYPR-OH | (Cpd. 118) |
| (SEQ ID NO. 118) | |
| H-P[Nle]E[Nle]RCSASVECKQKCLAAIGSIFGKC[Nle]NKKCKCYPR-OH | (Cpd. 119) |
| (SEQ ID NO. 119) | |
| H-P[Nle]E[Nle]RCSASVECKQKCLAAIGSIFGKC[Nle]NKKCKCYR-OH | (Cpd. 120) |
| (SEQ ID NO. 120) | |
| H-[Nle]E[Nle]RCSASVECKQKCLAAIGSIFGKC[Nle]NKKCKCYPR-OH | (Cpd. 121) |
| (SEQ ID NO. 121) | |
| H-P[Nle]E[Nle]RCSASVECKQKCLAAIGSIFGKC[Nle]NKKCKCYPRR- | (Cpd. 122) |
| OH (SEQ ID NO. 122) | |
| H-P[Nle]E[Nle]RCSASVECKQKCLAAIGSIFGKC[Nle]NKKCKCYPRY- | (Cpd. 123) |
| OH (SEQ ID NO. 123) | |
| H-P[Nle]E[Nle]RCSASVECKQKCLAAIGSIFGKC[Nle]NKKCKCYPRL- | (Cpd. 124) |
| OH (SEQ ID NO. 124) | |
| H-P[Nle]E[Nle]RCSASVECKQKCLAAIGSIFGKC[Nle]NKKCKCYPRH- | (Cpd. 125) |
| OH (SEQ ID NO. 125) | |
| H-P[Nle]E[Nle]RCSASVECKQKCLAAIGSIFGKC[Nle]NKKCKCYPRE- | (Cpd. 126) |
| OH (SEQ ID NO. 126) | |
| H-P[Nle]E[Nle]RCSASVECKQKCLAAIGSIFGKC[Nle]NKKCKCYPRKS- | (Cpd. 127) |

OH (SEQ ID NO. 127)

| | |
|---|---|
| H-P[Nle]E[Nle]RCSASVECKQKCLAAIGSIFGKC[Nle]NKKCKCYPRFE- | (Cpd. 128) |
| OH (SEQ ID NO. 128) | |
| H-P[Nle]E[Nle]RCSASVECKQKCLAAIGSIFGKC[Nle]NKKCKCYPRHR- | (Cpd. 129) |
| OH (SEQ ID NO. 129) | |
| H-P[Nle]E[Nle]RCSASVECKQKCLAAIGSIFGKC[Nle]NKKCKCYPRAK- | (Cpd. 130) |
| OH (SEQ ID NO. 130) | |
| H-P[Nle]E[Nle]RCSASVECKQKCLAAIGSIFGKC[Nle]NKKCKCYP-[(4- | (Cpd. 131) |
| amino-5-hydroxypentyl)guanidine] (SEQ ID NO. 131) | |
| H-P[Nle]E[Nle]RCSASVECKQKCLAAIGSIFGKC[Nle]NKKCKCYPR-[4- | (Cpd. 132) |
| amino-5-hydroxypentanamide] (SEQ ID NO. 132) | |
| H-P[Nle]E[Nle]RCSASVECKQKCLAAIGSIFGKC[Nle]NKKCKCYPRST- | (Cpd. 133) |
| OH (SEQ ID NO. 133) | |
| H-P[Nle]E[Nle]RCSASVECKQKCLAAIGSIFGKC[Nle]NKKCKCYPRRY- | (Cpd. 134) |
| OH (SEQ ID NO. 134) | |
| H-P[Nle]E[Nle]RCSSSVECKQKCLAAIGSIFGKC[Nle]NKKCKCYPR-OH | (Cpd. 135) |
| (SEQ ID NO. 135) | |
| H-P[Nle]E[Nle]RCSLSVECKQKCLAAIGSIFGKC[Nle]NKKCKCYPR-OH | (Cpd. 136) |
| (SEQ ID NO. 136) | |
| H-P[Nle]E[Nle]RCSAPVECKQKCLAAIGSIFGKC[Nle]NKKCKCYPR-OH | (Cpd. 137) |
| (SEQ ID NO. 137) | |
| H-P[Nle]E[Nle]RCSASPECKQKCLAAIGSIFGKC[Nle]NKKCKCYPR-OH | (Cpd. 138) |
| (SEQ ID NO. 138) | |
| H-P[Nle]E[Nle]RCSASQECKQKCLAAIGSIFGKC[Nle]NKKCKCYPR-OH | (Cpd. 139) |
| (SEQ ID NO. 139) | |
| H-P[Nle]E[Nle]RCSASVECLQKCLAAIGSIFGKC[Nle]NKKCKCYPR-OH | (Cpd. 140) |
| (SEQ ID NO. 140) | |
| H-P[Nle]E[Nle]RCSASVECKQPCLAAIGSIFGKC[Nle]NKKCKCYPR-OH | (Cpd. 141) |
| (SEQ ID NO. 141) | |
| H-P[Nle]E[Nle]RCEASVECKQKCLAAIGSIFGKC[Nle]NKKCKCYPR-OH | (Cpd. 142) |
| (SEQ ID NO. 142) | |
| H-P[Nle]E[Nle]RCFASVECKQKCLAAIGSIFGKC[Nle]NKKCKCYPR-OH | (Cpd. 143) |
| (SEQ ID NO. 143) | |
| H-P[Nle]E[Nle]RCSYSVECKQKCLAAIGSIFGKC[Nle]NKKCKCYPR-OH | (Cpd. 144) |
| (SEQ ID NO. 144) | |
| H-P[Nle]E[Nle]RCSAFVECKQKCLAAIGSIFGKC[Nle]NKKCKCYPR-OH | (Cpd. 145) |
| (SEQ ID NO. 145). | |

31. A nucleic acid encoding an ion channel blocker, a Kv1.3 inhibitor component, or a peptide Z¹—X—Z² as defined in claim 1, an expression vector comprising said nucleic acid, or a host cell comprising said nucleic acid or expression vector which is capable of expressing an ion channel blocker, a Kv1.3 inhibitor component, or a peptize Z¹—X—Z² as defined in claim 1.

32-33. (canceled)

34. A method of synthesising an ion channel blocker according to claim 1, the method comprising:
(a) synthesising the ion channel blocker by means of solid-phase or liquid-phase peptide synthesis methodology and recovering the peptide thus obtained;
(b) expressing the ion channel blocker from a nucleic acid construct that encodes the ion channel blocker and recovering the expression product; or
(c) expressing a precursor peptide from a nucleic acid construct that encodes the precursor peptide sequence, recovering the expression product, and modifying the precursor peptide to yield the ion channel blocker.

35. A pharmaceutical composition comprising an ion channel blocker or pharmaceutically acceptable salt according to claim 1, in admixture with a pharmaceutically acceptable carrier.

36. (canceled)

37. A method for
(i) inhibiting or reducing inflammation
(ii) treating an inflammatory condition or disorder;
(iii) inhibiting weight gain, promoting weight loss, or reducing excess body weight;
(iv) treating obesity, obesity-linked inflammation, obesity-linked gallbladder disease or obesity-induced sleep apnoea;
(v) treating a condition caused by or associated with impaired glucose control;
(vi) treating a smooth muscle proliferative disorder;
(vii) treating a neuroinflammatory or neurodegenerative disorder; or
(viii) treating cancer
in a subject in need thereof, wherein the method comprises administering the ion channel blocker or pharmaceutically acceptable salt of claim 1 to the subject.

38. (canceled)

39. The method according to claim 37 wherein
(i) the inflammatory condition or disorder is an autoimmune disorder, allergy or hypersensitivity, allograft rejection, or graft-versus-host disease;
(ii) the inflammatory condition or disease is hay fever, asthma, anaphylaxis, allergic rhinitis, urticaria, eczema, alopecia areata, dermatomyositis, inclusion body myositis, polymyositis, ankylosing spondylitis, vasculitis, arthritis (including rheumatoid arthritis, osteoarthritis, psoriatic arthritis), Sjogren's syndrome, systemic lupus erythematosus (SLE), uveitis, inflammatory fibrosis (e.g. scleroderma, lung fibrosis, cirrhosis), chronic obstructive pulmonary disease (COPD), hepatitis, chronic inflammatory demyelinating polyneuropathy, inflammatory bowel disease, colitis (e.g. Crohn's disease and ulcerative colitis), erythema, thyroiditis, psoriasis, atopic dermatitis, allergic contact dermatitis, scleroderma, glomerulonephritis, inflammatory bone resorption, multiple sclerosis, type 1 diabetes, transplant rejection or graft-versus-host disease;
(iii) the condition caused by or associated with impaired glucose control is metabolic syndrome, insulin resistance, glucose intolerance, pre-diabetes, increased fasting glucose or type 2 diabetes;
(iv) the smooth muscle proliferative disorder is restenosis;
(v) the neuroinflammatory or neurodegenerative disorder is Alzheimer's disease, multiple sclerosis (MS), Parkinson's disease or amyotrophic lateral sclerosis (ALS); or
(vi) the cancer is breast cancer, prostate cancer or lymphoma.

40-49. (canceled)

50. The method according to claim 39 wherein said lymphoma is non-Hodgkin lymphoma (NHL).

51. The method according to claim 50 wherein said NHL is large B-cell lymphoma, follicular lymphoma, Burkitt lymphoma, immunoblastic large cell lymphoma, precursor B-lymphoblastic lymphoma, mantle cell lymphoma, mycosis fungoides, anaplastic large cell lymphoma, peripheral T-cell lymphoma, precursor T-lymphoblastic lymphoma or Sezary syndrome.

52. An ion channel blocker or pharmaceutically acceptable salt according to claim 1, wherein any substitutions or deletions are made only at the following positions wherein, if different from PaT1:
the residue at position 1 is selected from N, P, V, H, Y and S or is deleted;
the residue at position 2 is selected from Nle and I or is deleted;
the residue at position 3 is selected from E and S or is deleted;
the residue at position 4 is selected from Nle, V, L, E and S or is deleted;
the residue at position 5 is K or is deleted;
the residue at position 7 is selected from K, R, E and F;
the residue at position 8 is selected from I, H, S, L and Y;
the residue at position 9 is selected from Abu, P, F, V and L;
the residue at position 10 is selected from R, P, Q and L;
the residue at position 11 is Q;
the residue at position 13 is selected from L, A, E, Q, V and G;
the residue at position 14 is selected from V, K, E, L and 2-amino-5-carboxypentanoyl;
the residue at position 15 is selected from S, L and P;
the residue at position 17 is selected from K, Y and R or is deleted;
the residue at position 18 is selected from D, A, Y, G, V, Q, hQ and L or is deleted;
the residue at position 19 is selected from R and Y or is deleted;
the residue at position 20 is selected from Y, E and R or is deleted;
the residue at position 21 is selected from R, H and E;
the residue at position 22 is selected from R and C;
the residue at position 23 is selected from R, K and P;
the residue at position 26 is homo-lysine;
the residue at position 28 is Nle;
the residue at position 30 is G;
the residue at position 33 is selected from H, V and R;
the residue at position 35 is selected from F(4-NH₂), F(4-F), F(4-NO₂) and F(4-CH₃);
the residue at position 36 is selected from Q, S and G or is deleted;
the residue at position 37 is selected from S and C or is deleted.
